(19) United States
(12) Patent Application Publication
(10) Pub. No.: US 2011/0166142 A1
(43) Pub. Date: Jul. 7, 2011

(54) 2-SUBSTITUTED ISOFLAVONOID COMPOUNDS, MEDICAMENTS AND USES

(76) Inventors: Eleanor Eiffe, New South Wales (AU); Andrew Heaton, New South Wales (AU); Catherine Walker, New South Wales (AU); Alan James Husband, New South Wales (AU)

(21) Appl. No.: 12/666,657

(22) PCT Filed: Jun. 30, 2008
(86) PCT No.: PCT/AU08/00960

§ 371 Date: Nov. 4, 2010

(30) Foreign Application Priority Data
Jun. 29, 2007 (AU) 2007903531

(51) Int. Cl. A61K 31/53; C07D 311/04; A61K 31/352; C07D 417/04; A61K 31/427; C07D 403/04; A61K 31/4192; C07D 401/04; A61K 31/4433; A61K 31/501; A61K 31/506; A61K 31/4965; A61K 31/4025; A61K 31/4178; A61K 31/41; A61P 1/04; A61P 29/00; A61P 11/06; A61P 19/02; A61P 9/00; A61P 3/06

(52) U.S. Cl. 514/242; 549/406; 514/456; 548/203; 514/365; 549/399; 549/404; 548/266.4; 514/359; 546/282.7; 514/337; 544/238; 514/252.01; 544/333; 514/256; 544/405; 514/255.05; 548/525; 514/422; 548/311.4; 514/397; 548/252; 514/382; 544/182; 544/179

(57) ABSTRACT

2-Substituted isoflavonoid compounds and pharmaceutical compositions containing same are useful as anti-inflammatory agents and antioxidants and for the treatment of related diseases and conditions.

### FIELD OF THE INVENTION

The present invention relates to 2-substituted isoflavonoid compounds and pharmaceutical compositions containing same useful as anti-inflammatory agents and antioxidants and for the treatment of related diseases and conditions. The invention further relates to novel 2-substituted isoflavonoid compounds and pharmaceutical compositions containing same.

### BACKGROUND OF THE INVENTION

Over 700 different naturally occurring isoflavones are known some of which have biological properties showing potential therapeutic benefit. However, despite the considerable research and accumulated knowledge in relation to isoflavones and derivatives thereof, the full ambit of therapeutically useful isoflavonoid compounds and their activities is yet to be realised. Moreover, there is a continual need for new, improved or at least alternative active agents for the treatment, prophylaxis, amelioration, defence against and/or prevention of various diseases and disorders, in particular inflammatory disorders and related conditions.

Inflammatory diseases and conditions include irritable bowel disease (IBD), for example, ulcerative colitis (UC), ulcerative proctitis, distal colitis and/or Crohn's disease (CD), as well as other hepatointestinal syndromes including primary sclerosing cholangitis (PSC), primary biliary cirrhosis (PBC), autoimmune hepatitis (AIH) and irritable bowel syndrome (IBS).

UC causes inflammation of the inner lining of the large bowel (colon and rectum). CD causes inflammation of the full thickness of the bowel wall and may involve any part of the digestive tract from the mouth to the anus. CD can cause recurrent bowel obstruction, fistulae, abscess formation and sepsis as well as extra-intestinal manifestations such as arthritis. IBD often develops between the ages of 15-30, and about 13,000 Australians have UC and 10,000 have CD. The Crohn's and Colitis Foundation of America estimates as many as one million Americans have IBD costing directly and indirectly around $US552 billion annually. Furthermore, there is research to suggest that persons with IBD are more likely to develop colon cancer.

The cause of IBD is unknown. However, both syndromes would appear to be immunologically-mediated and the inflammatory process is influenced by environmental and genetic factors. Medical therapy aims to control the inflammatory process, and is administered for active and chronic disease, as well as remission maintenance. No management strategy is totally effective, but current therapy is targeted at reducing inflammation and/or the immune response using anti-inflammatories (corticosteroids, aminosalicylates), immunosuppressives, immunotherapies or surgery. The goals of treatment are to induce and then maintain remission of disease, minimising the side-effects which accompany the therapies. Up to 80% of patients with UC and 35% in those with CD relapse within a year following the induction of remission (Podolsky, D. K. (2002) “The current future understanding of inflammatory bowel disease.” Best Practice & Research in Clinical Gastroenterology 16(6): 933-43). Additionally, none of the existing therapies are without significant side effects. The ‘holy grail’ in IBD drug development is therefore a non-toxic agent which will maintain remission of disease (Feagan 2003 “Maintenance therapy for inflammatory bowel disease” The American Journal of Gastroenterology 98 (12, Supplement 1): S6-S17).

Primary biliary cirrhosis (PBC), autoimmune hepatitis (A1H) and primary sclerosing cholangitis (PSC) are chronic liver diseases that are likely to have an autoimmune basis to their pathogenesis. PSC appears to be associated with UC. In PSC and PBC, the bile ducts become inflamed, scarred and eventually blocked, causing cholestasis, hepatocellular injury and in many cases liver failure.

Irritable Bowel Syndrome (IBS) is part of a spectrum of diseases known as Functional Gastrointestinal Disorders which include diseases such as non-cardiac chest pain, non-ulcer dyspepsia, and chronic constipation or diarrhoea. These diseases are all characterised by chronic or recurrent gastrointestinal symptoms for which no structural or biochemical cause can be found. IBS affects between 25 and 55 million people in the USA.

The prevalence of IBS in the general population of Western countries varies from 6 to 22%. IBS affects 14-24% of women and 5-19% of men. The prevalence is similar in Caucasians and African Americans, but appears to be lower in Hispanics. Although several studies have reported a lower prevalence of IBS among older people, the present studies do not allow to definitely conclude whether or not an age disparity exists in IBS. In non-Western countries such as Japan, China, India, and Africa IBS also appears to be very common.

Accordingly there is a need for new therapies in the treatment of inflammation and related diseases and conditions and new and improved agents and compounds useful for same.

2-Substituted compounds of the prior art are known from Grese et al., Tetrahedron Letters, Vol 36, No. 49, pp 8913-8916, (1995) and from EP 0 470 310-A1 and WO 93/10741. Compounds disclosed therein are said to be useful for the treatment of breast cancer.

### SUMMARY OF THE INVENTION

The present inventors have found a class of 2-substituted isoflavonoid compounds of the general formula (I) which exhibit important therapeutic activities including strong anti-inflammatory activity including inhibition of prostaglandins, thromboxanes and nitric oxide production, and anti-oxidante activity including free radical scavenging.

Thus according to an aspect of the present invention there is provided use of a 2-substituted isoflavonoid compound of the general formula (I):

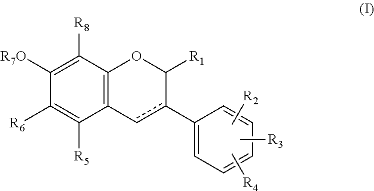

wherein
R₁ is hydroxy, OR₉, OC(O)R₉, OSi(R₁₀)₃, alkyl, cycloalkyl, aminoalkyl, —NR₁₁(R₁₂), R₁₁(R₁₂)N-alkyl, aryl, arylalkyl, thiol, alkylthio, nitro, cyano, halo, alkenyl, alkynyl, heteroaryl, arylalkylamino or alkylaryl,
R₂, R₃ and R₄ are independently hydrogen, hydroxy, OR₉, OC(O)R₉, OSi(R₁₀)₃, alkyl, cycloalkyl, aryl, arylalkyl, thiol, alkylthio, nitro, cyano or halo,
R₅, R₆ and R₈ are independently hydrogen, hydroxy, OR₉, OC(O)R₉ or alkyl,
R₇ is hydrogen, alkyl, haloalkyl, C(O)R₉, Si(R₁₀)₃, cycloalkyl, aryl or arylalkyl,
R₈ is hydrogen, alkyl, cycloalkyl, aryl, arylalkyl, nitro, cyano or halo,
R₉ is alkyl, haloalkyl, aryl or arylalkyl,
R₁₀ is independently alkyl or aryl,
R₁₁ and R₁₂ are independently hydrogen, alkyl, arylalkyl, aryl or BOC, or together form with the nitrogen atom to which they are attached a heterocyclic ring, and
the drawing [symbol] represents either a single bond or a double bond, preferably a double bond
   which hydrocarbon substituents can be optionally substituted by one or more of alkyl, halo, acyloxy, hydroxy, halo, alkoxy, silyloxy, nitro and cyano, and
   which compounds include pharmaceutically acceptable salts thereof,
   in the manufacture of a medicament as an anti-inflammatory agent or antioxidant.

According to another aspect of the present invention there is provided a method for the treatment, prevention or amelioration of an inflammatory disease or disorder, which comprises administering to a subject one or more compounds of the formula (I) or pharmaceutically acceptable salts or derivatives thereof, optionally in association with a carrier and/or excipient.

According to another aspect of the present invention there is provided the use of one or more compounds of formula (I) or pharmaceutically acceptable salts or derivatives thereof as an anti-inflammatory agent of antioxidant.

According to another aspect of the present invention there is provided an agent for the treatment, prophylaxis or amelioration of inflammation or as an antioxidant which agent comprises one or more compounds of formula (I), or pharmaceutically acceptable salts or derivatives thereof.

According to another aspect of the present invention there is provided a compound of formula (I) or a pharmaceutically acceptable salt or derivative thereof.

According to another aspect of the present invention there is provided a pharmaceutical composition which comprises one or more compounds of formula (I), or a pharmaceutically acceptable salt or derivative thereof in association with one or more pharmaceutical carriers, excipients, auxiliaries and/or diluents.

These and other aspects of the invention will become evident from the description and claims which follow, together with the accompanying drawings.

### BRIEF DESCRIPTION OF THE FIGURES

FIG. 1 shows the mean change of LPS-induced PGE₂ synthesis in RAW 264.7 murine macrophages by test compounds at 10 μM relative to treatment vehicle alone.

FIG. 2 shows the mean change of LPS-induced TXB₂ synthesis in RAW 264.7 murine macrophages by test compounds at 10 μM relative to treatment vehicle alone.

FIG. 3 shows the mean change of LPS-induced NO synthesis in RAW 264.7 murine macrophages by test compounds at 10 μM relative to treatment vehicle alone.

### DETAILED DESCRIPTION OF THE INVENTION

The present inventors have found that a class of 2-substituted isoflavonoid compounds of the general formula (I) show important biological and pharmaceutical properties.

The compounds of formula (I) possess the ability to inhibit inflammatory processes and to moderate immunological processes. The compounds are therefore also useful in the prevention and treatment of disorders generally recognised as being associated with excessive inflammation or dysfunctional immune function and embracing but not limited to inflammatory conditions of the gastrointestinal tract including inflammatory bowel disease (including ulcerative colitis and Crohn's disease) sclerosing cholangitis, inflammatory disorders of synovial membranes, including rheumatoid arthritis, inflammatory conditions of the respiratory system, including asthma as well as autoimmune diseases including glomerulonephritis. The compounds are also useful in the treatment of diseases involving pulmonary inflammation, cardiovascular disorders including atherosclerosis, hypertension and lipid dyscrasias. The compounds of formula (I) may also be useful in the treatment of pain associated with inflammation and/or are thought to be cardioprotective or gut protective due to their action as a selective thromboxane synthesis inhibitor.

The properties described above offer significant clinical advantages.

According to another aspect of the invention, there is provided a 2-substituted isoflavonoid compound of the general formula (I):

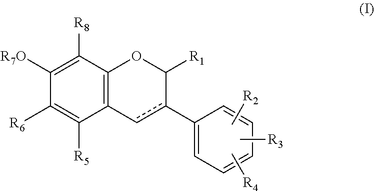

wherein
R₁ is hydroxy, OR₉, OC(O)R₉, alkyl, cycloalkyl, aminoalkyl, —NR₁₁(R₁₂), R₁₁(R₁₂)N-alkyl, aryl, arylalkyl, thiol, alkylthio, nitro, cyano, halo, alkenyl, alkynyl, heteroaryl, arylalkylamino or alkylaryl,
R₂, R₃ and R₄ are independently hydrogen, hydroxy, OR₉, OC(O)R₉, alkyl, cycloalkyl, aryl, arylalkyl, thiol, alkylthio, nitro, cyano or halo,
R₅, R₆ and R₈ are independently hydrogen, hydroxy, OR₉, OC(O)R₉ or alkyl,
R₇ is hydrogen, alkyl, haloalkyl, C(O)R₉, cycloalkyl, aryl or arylalkyl,
R₈ is hydrogen, alkyl, cycloalkyl, aryl, arylalkyl, nitro, cyano or halo,
R₉ is alkyl, haloalkyl, aryl or arylalkyl,
R₁₁ and R₁₂ are independently hydrogen, alkyl, arylalkyl, aryl or BOC, or together form with the nitrogen atom to which they are attached a heterocyclic ring, and
the drawing [symbol] represents either a single bond or a double bond, preferably a double bond
   which hydrocarbon substituents can be optionally substituted by one or more of alkyl, halo, acyloxy, hydroxy, halo, alkoxy, silyloxy, nitro and cyano, and
   which compounds include pharmaceutically acceptable salts thereof.

In a preferred embodiment of the invention there is provided a 2-substituted isoflav-3-ene compound of the formula (I-1):

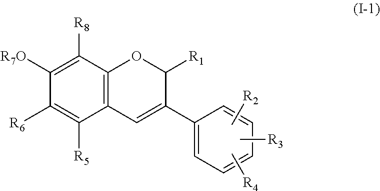

wherein
R₁ is hydroxy, OR₉, OC(O)R₉, alkyl, cycloalkyl, aminoalkyl, —NR₁₁(R₁₂), R₁₁(R₁₂)N-alkyl, aryl, arylalkyl, thiol, alkylthio, nitro, cyano, halo, alkenyl, alkynyl, heteroaryl, arylalkylamino or alkylaryl,
R₂, R₃ and R₄ are independently hydrogen, hydroxy, OR₉, OC(O)R₉, alkyl, cycloalkyl, aryl, arylalkyl, thiol, alkylthio, nitro, cyano or halo,
R₅, R₆ and R₈ are independently hydrogen, hydroxy, OR₉, OC(O)R₉ or alkyl,
R₇ is hydrogen, alkly or haloalkyl, C(O)R₉, cycloalkyl, aryl or arylalkyl,
R₉ is alkyl, haloalkyl, aryl or arylalkyl,
R₁₀ is independently alkyl or aryl, and
R₁₁ and R₁₂ are independently hydrogen, alkyl, arylalkyl, aryl or BOC, or together with the nitrogen atom to which they are attached form a heterocyclic ring,
   which hydrocarbon substituents can be optionally substituted by one or more of alkyl, halo, acyloxy, hydroxy, halo, alkoxy, silyloxy, nitro and cyano, and
   which compounds include pharmaceutically acceptable salts thereof.

In one embodiment, R₁ is an alkyl group selected from methyl, ethyl, propyl, isopropyl, n-butyl and t-butyl. In further embodiment, R₁ is selected from propyl, n-butyl and t-butyl.

In another embodiment, R₁ is a haloalkyl group selected from trifluoromethyl.

In another embodiment, R₁ is an aminoalkyl group selected from aminomethyl.

In another embodiment, R₁ is an alkenyl group selected from allyl.

In another embodiment, R₁ is an akynyl group selected from ethynyl.

In another embodiment, R₁ is an alkoxy group selected from methoxy, ethoxy and bromopropoxy.

In another embodiment, R₁ is an amino group selected from benzylamino.

In another embodiment, R₁ is cyano.

In another embodiment, R₁ is hydroxy.

In another embodiment, R₁ is an alkylthio group selected from methylthio and ethylthio.

In another embodiment, R₁ is a heteroaryl group selected from thiazolyl, triazolyl, pyridinyl, pyridazyl, pyrimidinyl, pyrazinyl, pyrrolyl, imidazyl, triazolyl, tetrazolyl, triazinyl and tetrazinyl.

In a further preferred embodiment, the compound is of formula (I-1) wherein

R₂, R₃ and R₄ are independently hydrogen, hydroxy, OR₉, OC(O)R₉ or halo, more preferably, wherein
R₂, R₃ and R₄ are independently hydrogen, hydroxy, OMe or OC(O)Me.

In a preferred embodiment of the invention there is provided a 2-substituted isoflav-3-ene compound of the formula (I-2):

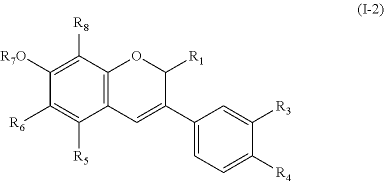

wherein
R₃ and R₄ are independently hydrogen, hydroxy, methoxy or OC(O)Me, and
more preferably, wherein
R₃ and R₄ are independently hydrogen, hydroxy or methoxy, and
more preferably wherein
one of R₃ and R₄ is hydroxy and the other is hydrogen.

In a further preferred embodiment, the compound is of formula (I-1) or (I-2) wherein

R₅, R₆ and R₈ are independently hydrogen, hydroxy or methyl, and
more preferably, wherein
one of R₅, R₆ and R₈ are hydroxy or methyl,
more preferably, wherein
R₅, R₆ and R₈ are hydrogen.

In a further preferred embodiment, the compound is of formula (I-1) or (I-2) wherein

R₇ is hydrogen, methyl or C(O)Me,
more preferably, wherein
R₇ is hydrogen.

In a further preferred embodiment, the compound is a compound of formula (I-1) or (I-2)

wherein
R₁ is heteroaryl,

### R₂ is H,

R₃ and R₄ are independently hydrogen, hydroxy or methoxy,
R₅, R₆ and R₈ are independently hydrogen, hydroxy or methyl, and
R₇ is hydrogen or methyl.

In a further preferred embodiment, the compound is a compound of formula (I-1) or (I-2)

wherein
R₁ is a 5 or 6-membered aromatic ring wherein between 1 and 3 atoms are nitrogen,

### R₂ is H,

R₃ and R₄ are independently hydrogen, hydroxy or methoxy,
R₅, R₆ and R₈ are independently hydrogen, hydroxy or methyl, and
R₇ is hydrogen.

In a further preferred embodiment, the compound is a compound of formula (I-1) or (I-2)

wherein
R₁ is pyridyl, pyrimidinyl, pyrazinyl or pyridazinyl,

### R₂ is H,

R₃ and R₄ are independently hydrogen, hydroxy or methoxy,
R₅, R₆ and R₈ are independently hydrogen, hydroxy or methyl, and
R₇ is hydrogen or methyl.

Especially preferred compounds of formula (I) are compounds (1) to (38):

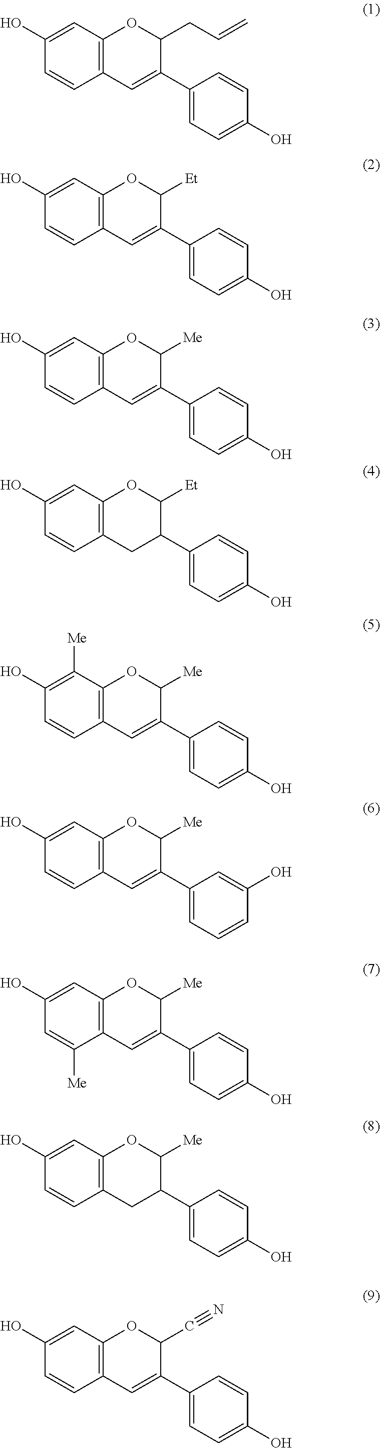

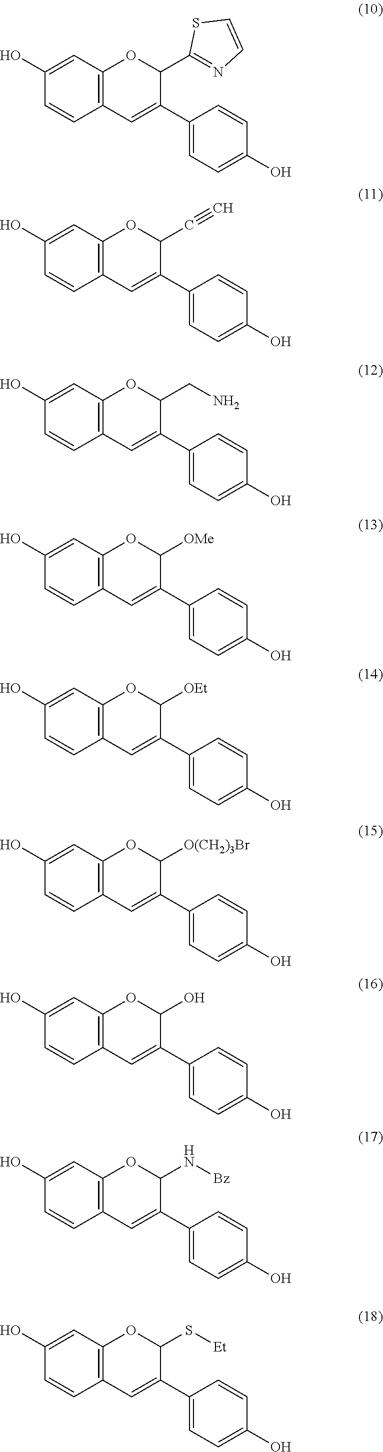

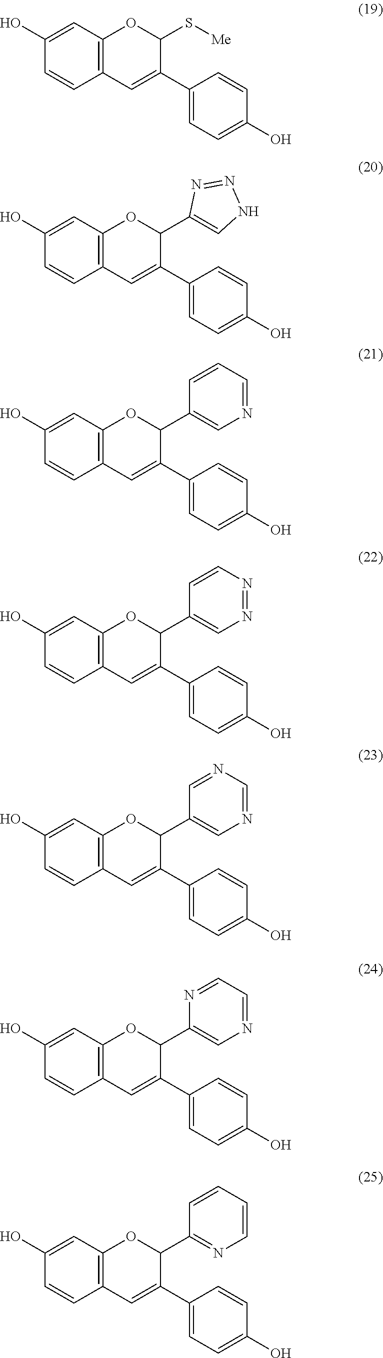

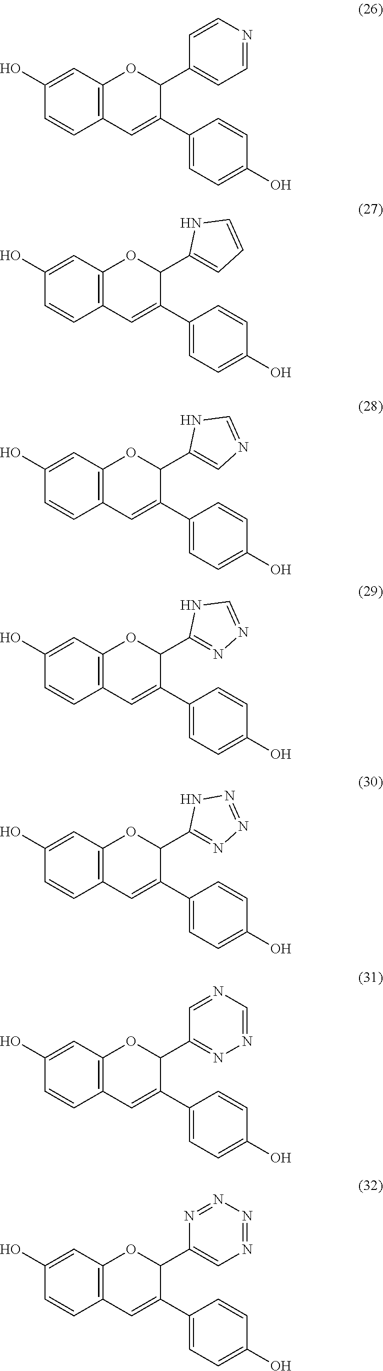

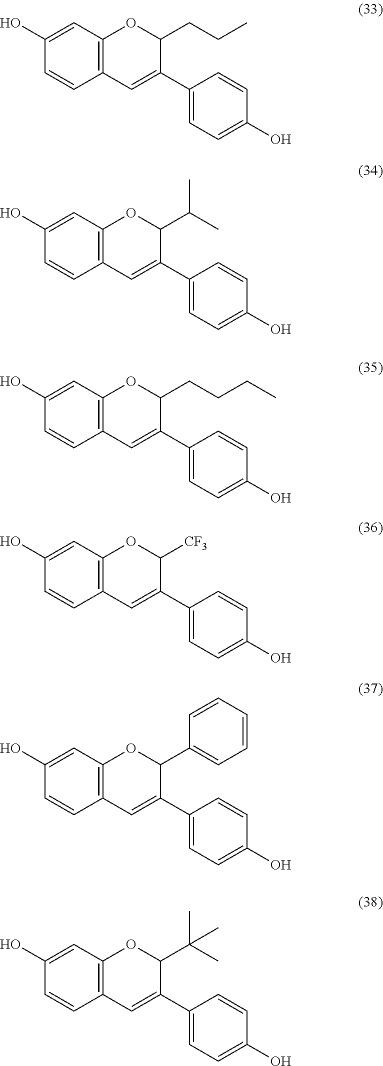

or a pharmaceutically acceptable salt thereof.

These compounds may also be present as corresponding hydroxyl-protected compounds, including acetoxy-protected (compounds 39 to 76) and trimethylsilyl-protected (compounds 77 to 114).

The 3-ene compounds (1)-(3), (5)-(7) and (9)-(38) find particular utility in the methods of the invention. Particular mention can be made of compounds (1), (5), (6), (7), (13), (14) and (18). Additional mention can be made of compounds (10) and (20)-(32).

In as much as compounds are known from the prior art, they do not form part of the invention as it relates to novel compounds. In a preferred aspect of the invention there is provided a 2-substituted isoflav-3-ene or isoflavan compound of formula (I) with the proviso that the following compounds are specifically excluded:
2-methyl-4′,7-dihydroxyisoflav-3-ene,
2-ethyl-4′,7-dihydroxyisoflav-3-ene,
2-isopropyl-4′,7-dihydroxyisoflav-3-ene,
2-phenyl-4′,7-dihydroxyisoflav-3-ene,
2-(4-fluorophenyl)-4′,7-dihydroxyisoflav-3-ene,
2-(4-anisyl)-4′,7-dihydroxyisoflav-3-ene,
2-naphthyl-4′,7-dihydroxyisoflav-3-ene,
2-thienyl-4′,7-dihydroxyisoflav-3-ene,
2-vinyl-4′,7-dihydroxyisoflav-3-ene,
2-(4-hydroxyphenyl)-3-phenyl-7-methoxy-2H-1-benzopyran, and
2-(N-n-butyl-N-methyl-10-aminodecyl)-3(4-hydroxyphenyl)-7-hydroxy-2H-1-benzopyran.

The compounds of formula (I) according to the invention can have chiral centres. The present invention includes all the enantiomers and diastereoisomers as well as mixtures thereof in any proportions. The invention also extends to isolated enantiomers or pairs of enantiomers. Methods of separating enantiomers and diastereoisomers are well known to persons skilled in the art.

The term “isoflavone” or “isoflavonoid compound” as used herein is to be taken broadly to include as isoflavones, isoflavenes, isoflavans, isoflavanones, isoflavanols and the like where a specific meaning is not intended.

The term “alkyl” is taken to include straight chain and branched chain monovalent saturated hydrocarbon groups having 1 to 6 carbon atoms, such as methyl, ethyl, propyl, isopropyl, butyl, isobutyl, secbutyl, tertiary butyl, pentyl and the like. The alkyl group more preferably contains from 1 to 4 carbon atoms, especially methyl, ethyl, propyl or isopropyl.

The term “alkenyl” is taken to include straight chain and branched chain monovalent hydrocarbon radicals having 2 to 6 carbon atoms and at least one carbon-carbon double bond, such as vinyl, propenyl, 2-methyl-2-propenyl, butenyl, pentenyl and the like. The alkenyl group may contains from 2 to 4 carbon atoms.

The term “alkynyl” is taken to include straight chain and branched chain monovalent hydrocarbon radicals having 2 to 6 carbon atoms and at least one carbon-carbon triple bond, such as ethynyl, propynyl, butynyl, pentynyl, hexynyl and the like. The alkynyl group may contain from 2 to 4 carbon atoms.

Cycloalkyl includes cyclic alkyl groups of 3 to 6 carbon atoms such as cyclopropyl, cyclobutyl, cyclopentyl and cyclohexyl.

The alkyl, alkenyl or alkynyl groups or cycloalkyl group may optionally be substituted by one or more of alkyl, halo, acyloxy, hydroxy, halo, alkoxy, silyloxy, nitro and cyano.

The term “aryl” is taken to include phenyl, benzyl, biphenyl and naphthyl and may be optionally substituted by one or more of alkyl, halo, acyloxy, hydroxy, halo, alkoxy, silyloxy, nitro and cyano.

The term “heteroaryl” is taken to mean a monovalent aromatic radical having between 1 and 12 atoms, wherein 1 to 6 atoms are heteroatoms selected from nitrogen, oxygen and sulfur. “Heteroaryl” may be taken to mean a monovalent aromatic radical having between 1 and 6 atoms, wherein 1 to 4 or 1 to 3 atoms are heteroatoms selected from nitrogen, oxygen and sulfur. The heteroaryl group may have 5 or 6 atoms, wherein 1 to 3 or 1 to 4 atoms are heteroatoms selected from oxygen, nitrogen and sulfur. The heteroaryl group may be selected from the group consisting of: furanyl, tetrazinyl, pyrazolyl, tetrazolyl, oxazolyl, isoxazolyl, isothiazolyl, thiazolyl, thienyl, imidazolyl, pyrazinyl, pyridazinyl, pyrimidinyl, pyridyl, pyrrolyl, triazolyl and triazinyl. The heteroaryl group may be selected from the group consisting of: pyridyl, pyrimidinyl, pyrazinyl, pyridazinyl and pyrimidinyl. The heteroaryl group may be 1-pyridyl, 2-pyridyl or 3-pyridyl. The heteroaryl group may be optionally substituted by one or more of alkyl, halo, acyloxy, hydroxy, halo, alkoxy, silyloxy, nitro and cyano.

Preferred heteroaryl groups are furanyl, tetrazinyl, pyrazolyl, tetrazolyl, oxazolyl, isoxazolyl, isothiazolyl, thiazolyl, imidazolyl, pyrazinyl, pyridazinyl, pyrimidinyl, pyridyl, pyrrolyl, triazolyl or triazinyl.

The term “halo” is taken to include fluoro, chloro, bromo and iodo, preferably fluoro and chloro. Reference to for example “haloalkyl” will include monohalogenated, dihalogenated and up to perhalogenated alkyl groups. Preferred perhaloalkyl groups are trifluoromethyl and pentafluoroethyl.

The term “aminoalkyl” means alkyl as defined above, wherein one or more hydrogen atoms have been replaced by one or more amino groups. One or two hydrogen atoms may be replaced by one or two amino groups. The aminoalkyl group may be aminomethyl, aminoethyl, aminopropyl and the like.

The term “arylalkyl” means an aryl group as defined above attached to the molecule via a divalent alkylene group. Examples of arylalkyl groups include benzyl and phenethyl and the like. The term “alkylene” means a divalent group derived from a straight or branched chain saturated hydrocarbon group by the removal of two hydrogen atoms. Representative alkylene groups include methylene, ethylene, propylene, isobutylene, and the like.

The term “alkylaryl” means an alkyl group as defined above attached to the molecule via a divalent arylene group. Examples of alkylaryl groups include tolyl, ethylphenyl, propylphenyl, butylphenyl and the like. The term “arylene” means an aromatic ring system derived from an aryl group as defined above by the removal of two hydrogen atoms.

The term “silyloxy” group typically refers to peralkylsilyloxy such as trimethylsilyloxy or t-butyldimethylsilyloxy.

The compounds of the invention include all salts, such as acid addition salts, anionic salts and zwitterionic salts, and in particular include pharmaceutically acceptable salts as would be known to those skilled in the art. The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, tosylate, citrate, bicarbonate and carbonate.

Pharmaceutically acceptable salts include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, p-toluenesulphonic, tartaric, trifluoroacetic, triphenylacetic, tricarballylic, salicylic, sulphuric, sulphamic, sulphanilic and succinic acid.

The term “pharmaceutically acceptable derivative” or “prodrug” refers to a derivative of the active compound that upon administration to the recipient is capable of providing directly or indirectly, the parent compound or metabolite, or that exhibits activity itself and includes for example phosphate derivatives and sulphonate derivatives. Thus, derivatives include solvates, pharmaceutically active esters, prodrugs or the like. This also includes derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

Chemical functional group protection, deprotection, synthons and other techniques known to those skilled in the art may be used where appropriate to aid in the synthesis of the compounds of the present invention, and their starting materials.

The protection of functional groups on the compounds and derivatives of the present invention can be carried out by well established methods in the art, for example as described in T. W. Greene, Protective Groups in Organic Synthesis, John Wiley & Sons, New York, 1981.

Hydroxy protecting groups include but are not limited to carboxylic acid esters, eg acetate esters, aryl esters such as benzoate, acetals/ketals such as acetonide and benzylidene, ethers such as o-benzyl and p-methoxy benzyl ether, tetrahydropyranyl ether and silyl ethers such as trimethylsilyl and t-butyldimethylsilyl ethers.

Protecting groups can be removed by, for example, acid or base catalysed hydrolysis or reduction, for example, hydrogenation. Silyl ethers may require hydrogen fluoride or tetrabutylammonium fluoride to be cleaved.

It will be clear to persons skilled in the art of medicinal chemistry that compounds of formula (I) may be converted into other compounds of formula (I), for example, where a compound of formula (I) bears one or more hydroxyl substituents then one or more of these substituents can be converted in to a halo substituent such as bromo, chloro or iodo by treating the alcohol with a halogenating agent. Halogenating agents include compounds like NBS, hydrobromic acid, chlorine gas etc. It may be necessary during processes such as halogenation to use protecting groups to protect other functionality in the molecule.

Phenolic type hydroxyls may not be readily convertible to the corresponding halogen compound by treatment with a halogenating agent. However, the desired halogen compound may be prepared by, for example, treating an appropriate aryl amine starting material with NaNO₂ in the presence of HCl under reduced temperature conditions such as 0° C., to form the corresponding azide salt. Subsequent treatment with CuCl, CuBr, KI or HBF₄ may be used to convert the azide into the required halo-compound.

The 2-substituted isoflav-3-enes were synthesized from protected isoflav-3-enes using trityl hexafluorophosphate in forming the corresponding isoflavylium salt intermediate and this was followed by nucleophilic addition. Scheme 1 below depicts the general synthetic methodology.

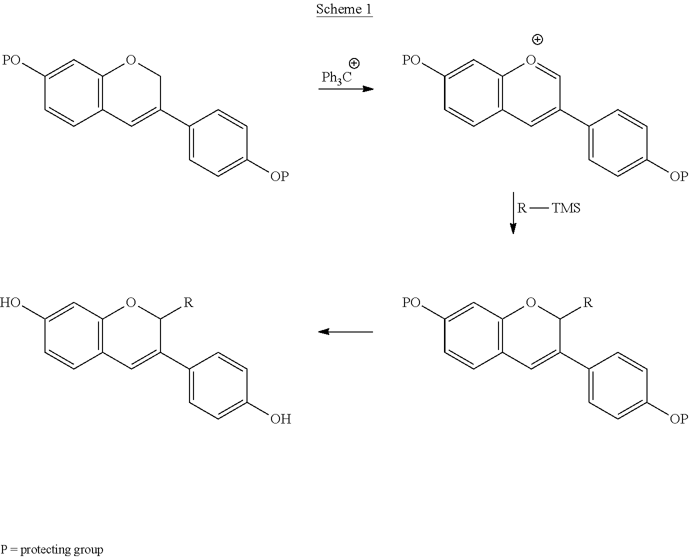

Nucleophiles utilised included trimethyl silyl (TMS) derivatives, a tributyl tin ((Bu)₃Sn) derivative, alcohols and amines as necessary and with optional functional group modification as would be known to one skilled in the art to arrive at the compounds of the invention.

Thus according to another aspect of the present invention there is provided a process for the preparation of a compound of formula (I):

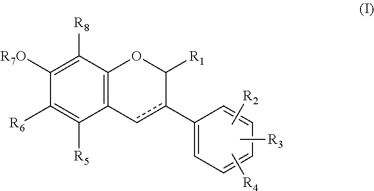

wherein
R₁, R₂, R₃, R₄, R₅, R₆, R₇ and R₈ are as defined above, and
the drawing [symbol] represents either a single bond or a double bond,
which hydrocarbon substituents can be optionally substituted by one or more of alkyl, halo, acyloxy, hydroxy, halo, alkoxy, silyloxy, nitro and cyano,
comprising the step of reacting an isoflavylium salt of formula (II):

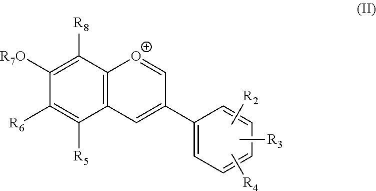

wherein R₂, R₃, R₄, R₅, R₆, R₇ and R₈ and are suitably protected,
with a nucleophile R₁—X
wherein R₁ is nucleophilic and X is a counter group including TMS, (Bu)₃Sn or H, formaldehyde and a primary amine, R₁—NH₂,
to form a compound of the general formula (I).

Access to various substitution patterns around both the benzopyran ring and the pendant phenyl ring of the 2-substituted compounds is made possible by selecting correspondingly substituted R₅-R₈-phenols and R₂-R₄-phenyl acetic acid starting materials according to, for example, published International application Nos. WO 98/08503 and WO01/17986, and references cited therein, the disclosures of which are incorporated herein by reference. A typical synthesis is depicted in Scheme 2 below.

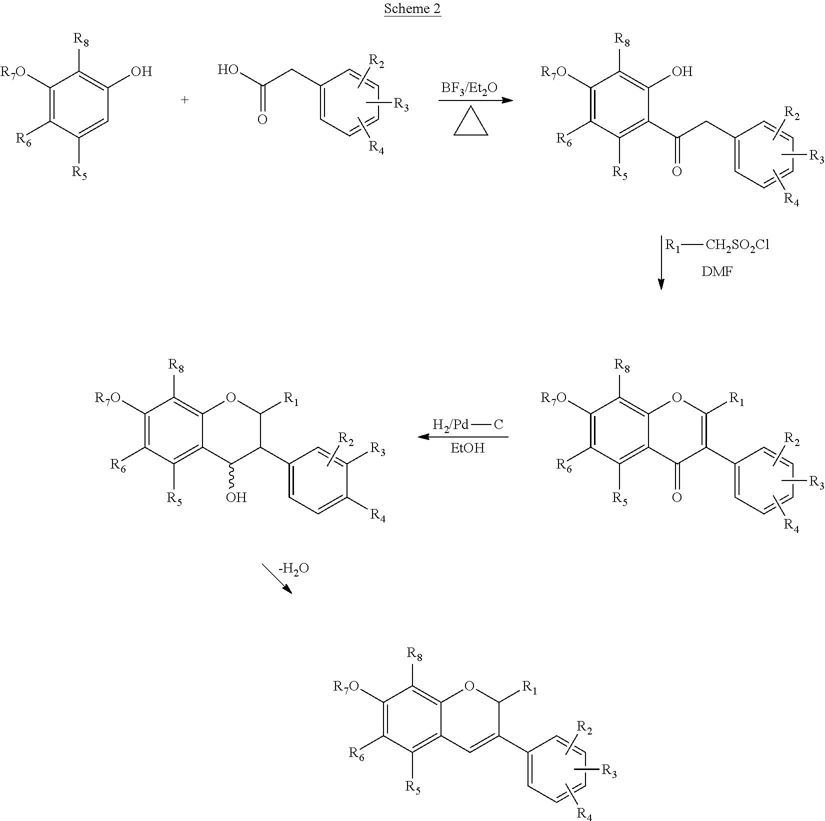

The ring cyclisation reactions my conveniently be preformed with R₁-substituted methanesulfonyl chloride reactants as would be known to those skilled in the art. R₁ may be protected or present as an Umpoled synthon as appropriate. The reduction reaction is successfully performed with Pd—C or Pd-alumina in an alcoholic solvent in the presence of hydrogen to afford isoflavan-4-ol compounds. Dehydration may be effected with acid or P₂O₅ for example. The hydrogenation and dehydration reactions generally work better when the phenol moieties when present are first protected, such as for example as acyloxy or silyloxy groups. The products can then be readily deprotected to generate the corresponding hydroxy-substituted compounds. Persons skilled in the art will be aware that other standard methods of alkylation, cyclisation, hydrogenation and/or dehydration can be employed as appropriate.

Where R₁ is hydrogen, the isoflavylium salt method from Scheme 1 can be employed to effect insertion of the R₁ group.

Access to the various 3-phenyl substituted isoflavones is available by varying the substitution pattern on the phenylacetic acid derived group, or chemical modification thereof with protecting groups and synthons as known in the art.

Likewise, access to the 5-, 6- and/or the 8-substituted isoflavones is available by varying the substitution pattern on the resorcinol group.

Access to 2-substituted isoflavonoids is also available by anhydride cyclisation with 1,2-diphenyl-ethanones to afford isoflavones, followed by hydrogenation and subsequent dehydration of the resultant 4-ol to afford isoflav-3-enes of the subject invention. Scheme 3 below depicts the general synthetic methodology.

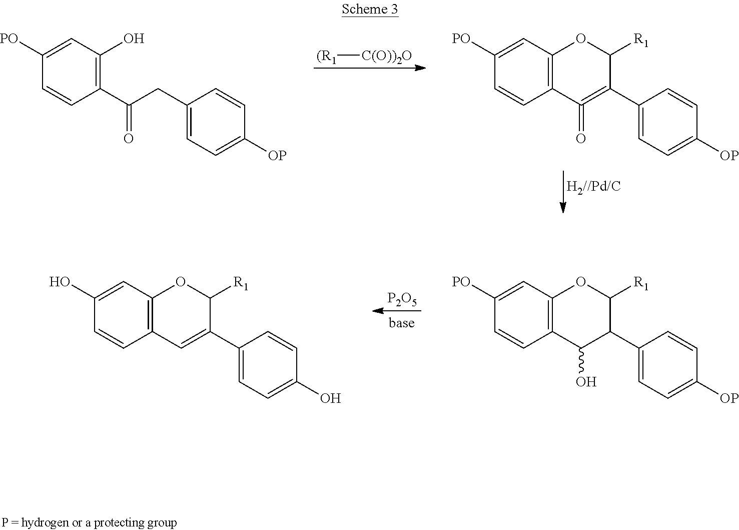

Access to various 2-substituted compounds is obtained by varying the acid anhydride group. Access to various substitution patters around the pendant phenyl ring and the benzopyran phenyl ring is possible by starting with correspondingly substituted 1,2-diphenyl ethanones.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The amount of one or more compounds of formula (I) which is required in a therapeutic treatment according to the invention will depend upon a number of factors, which include the specific application, the nature of the particular compound used, the condition being treated, the mode of administration and the condition of the patient.

Compounds of formula (I) may be administered in a manner and amount as is conventionally practised. See, for example, Goodman and Gilman, “The pharmacological basis of therapeutics”, 7th Edition, (1985). The specific dosage utilised will depend upon the condition being treated, the state of the subject, the route of administration and other well known factors as indicated above. In general, a daily dose per patient may be in the range of 0.1 mg to 5 g; typically from 0.5 mg to 1 g; preferably from 50 mg to 200 mg. The length of dosing may range from a single dose given once every day or two, to twice or thrice daily doses given over the course of from a week to many months to many years as required, depending on the severity of the condition to be treated or alleviated.

It will be further understood that for any particular subject, specific dosage regimens should be adjusted over time according to the individual need and the professional judgment of the person administering or supervising the administration of the compositions.

Relatively short-term treatments with the active compounds can be used to cause stabilisation or shrinkage or remission of cancers. Longer-term treatments can be employed to prevent the development of cancers in high-risk patients.

The production of pharmaceutical compositions for the treatment of the therapeutic indications herein described are typically prepared by admixture of the compounds of the invention (for convenience hereafter referred to as the “active compounds”) with one or more pharmaceutically or veterinary acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain up to 100% by weight of the active compound, preferably from 0.5% to 99%, or from 0.5% to 85%, or from 0.5% to 75%, or from 0.5 to 60% by weight of the active compound.

One or more active compounds may be incorporated in the formulations of the invention, which may be prepared by any of the well known techniques of pharmacy consisting essentially of admixing the components, optionally including one or more accessory ingredients. The preferred concentration of active compound in the drug composition will depend on absorption, distribution, inactivation, and excretion rates of the drug as well as other factors known to those of skill in the art.

The formulations of the invention include those suitable for oral, rectal, ocular, buccal (for example, sublingual), parenteral (for example, subcutaneous, intramuscular, intradermal, or intravenous), transdermal administration including mucosal administration via the nose, mouth, vagina or rectum, and as inhalants, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more other ingredients.

Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatine and glycerin or sucrose and acacia.

Formulations suitable for ocular administration include liquids, gels and creams comprising the active compound in an ocularly acceptable carrier or diluent.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and can be administered at a rate of 0.1 ml/minute/kg.

Formulations for infusion, for example, may be prepared employing saline as the carrier and a solubilising agent such as a cyclodextrin or derivative thereof. Suitable cyclodextrins include α-cyclodextrin, β-cyclodextrin, γ-cyclodextrin, dimethyl-β-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. More preferably the cyclodextrin is hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Formulations suitable for rectal administration are preferably presented as unit dose suppositories. Formulations suitable for vaginal administration are preferably presented as unit dose pessaries. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vasoline, lanoline, polyethylene glycols, alcohols, and combination of two or more thereof. The active compound is generally present at a concentration of from 0.1% to 5% w/w, more particularly from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. See for example Brown, L., et al. (1998).

Formulations suitable for transdermal administration may also be delivered by iontophoresis (see, for example, Panchagnula R, et al., 2000) and typically take the form of an optionally buffered aqueous solution of the active compound. Suitable formulations comprise citrate or Bis/Tris buffer (pH 6) or ethanol/water and contain from 0.1 M to 0.2 M active ingredient.

Formulations suitable for inhalation may be delivered as a spray composition in the form of a solution, suspension or emulsion. The inhalation spray composition may further comprise a pharmaceutically acceptable propellant such as carbon dioxide or nitrous oxide or a hydrogen containing fluorocarbon such as 1,1,1,2-tetrafluoroethane, 1,1,1,2,3,3,3-heptafluoro-n-propane or mixtures thereof.

The active compounds may be provided in the form of food stuffs, such as being added to, admixed into, coated, combined or otherwise added to a food stuff. The term “food stuff” is used in its widest possible sense and includes liquid formulations such as drinks including dairy products and other foods, such as health bars, desserts, etc. Food formulations containing compounds of the invention can be readily prepared according to standard practices.

Therapeutic methods, uses and compositions may be for administration to humans or other animals, including mammals such as companion and domestic animals (such as dogs and cats) and livestock animals (such as cattle, sheep, pigs and goats), birds (such as chickens, turkeys, ducks), marine animals including those in the aquaculture setting (such as fish, crustaceans and shell fish) and the like.

The active compound or pharmaceutically acceptable derivatives prodrugs or salts thereof can also be co-administered with other active materials that do not impair the desired action, or with materials that supplement the desired action, such as antibiotics, antifungals, antiinflammatories, or antiviral compounds. The active agent can comprise two or more isoflavones or derivatives thereof in combination or synergistic mixture. The active compounds can also be administered with lipid lowering agents such as probucol and nicotinic acid; platelet aggregation inhibitors such as aspirin; antithrombotic agents such as coumadin; calcium channel blockers such as verapamil, diltiazem, and nifedipine; angiotensin converting enzyme (ACE) inhibitors such as captopril and enalapril, and β-blockers such as propanolol, terbutalol, and labetalol. The compounds can also be administered in combination with nonsteriodal antiinflammatories such as ibuprofen, indomethacin, aspirin, fenoprofen, mefenamic acid, flufenamic acid and sulindac. The compounds can also be administered with corticosteroids or an anti-emetic such as Zofran®.

Compounds of formula (I) seem to be particularly suitable for co-administration with one or more anti-cancer drugs such as cisplatin, dehydroequol, taxol (paclitaxel), gemcitabine, doxorubicin, topotecan and/or camptothecin. This may result in improved effects in the treatment, for example in the form of synergistic effects, in comparison to when only one of the medicaments is employed. Particularly the compounds of the presently claimed invention seem to be chemosensitisers and increase the cytotoxicity of the one or more anticancer drug co-administered therewith. This seems to be the case even though said anticancer drugs work through a variety of different mechanisms, for example cisplatin is thought to work by interacting with nuclear DNA, taxol is thought to work by blocking cells in the G2/M phase of the cell cycle and prevent them forming normal mitotic apparatus, gemcitabine is thought to work by incorporating itself into the DNA of the cell, ultimately preventing mitosis, doxorubicin is though to be a topoisomerase II inhibitor thereby preventing DNA replication and transcription and topotecan is thought to be a topoisomerase I inhibitor.

Interestingly, in some situations this increased cytotoxicity to cancerous cells is not associated with a corresponding increase in toxicity to non-cancerous cells. Whilst this observation has important implications for the treatment of many cancers, it is especially important to the treatment of cancers such as melanoma, which are extremely difficult to treat.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

The invention also extends to a pack comprising the combination therapy.

The compounds of formula (I) are also found to be cytostatic and cytotoxic against a broad range of cancer cells of human and animal origin. By cancer cells, it is meant cells that display malignant characteristics and which are distinguished from non-cancer cells by unregulated growth and behaviour which usually ultimately is life-threatening unless successfully treated.

The cancer cells that have been found to be responsive to compounds of formula (I) are of epithelial origin (for example, prostate, ovarian, cervical, breast, gall-bladder, pancreatic, colorectal, renal, and non-small lung cancer cells), of neural origin (for example glioma cancer cells) and of mesenchymal origin (for example, melanoma, mesothelioma and sarcoma cancer cells). It is highly unusual and surprising to find a related group of compounds that display such potent cytotoxicity against cancer cells, but with generally lower toxicity against non-cancer cells such as fibroblasts derived from human foreskin. Such cancer cell selectivity is highly unusual and unexpected.

Advantageously the compounds of formula (I) show cytotoxic activity against cancer cells that are well recognised for being poorly sensitive to standard anti-cancer drugs.

The invention also provides the use of compounds of formula (I) to treat patients with cancer by either reducing the rate of growth of such tumours or by reducing the size of such tumours through therapy with said compounds alone, and/or in combination with each other, and/or in combination with other anti-cancer agents, and/or in combination with radiotherapy.

The use of compounds of the present invention either alone or in combination therapy as described above may reduce the adverse side-effects often experienced by patients when treated with standard anti-cancer treatments. The use of compounds of the invention may mean that lower doses can be employed in such therapy which represents an important advance for individuals with cancer.

The compounds of formula (I) of the invention are shown to have favourable toxicity profiles with normal cells and good bioavailability. The compounds of the invention exhibit anti-cancer activity significantly better than, comparable to or at least as a useful alternative to existing cancer treatments.

A requirement accordingly exists for new generation compounds that exhibit physiological properties important to the health and well-being of animals, particularly humans, and to find new methods which exploit these properties for the treatment, amelioration and prophylaxis of disease. Importantly, there is a strong need to identify new, improved, better and/or alternative pharmaceutical compositions, agents and regimes for compounds active against the proliferation of cells including cancer and related diseases. There is a further need for chemotherapeutic agents which address some of the undesirable side effects of known agents. There is also a need for different therapies to be available to physicians to combat the numerous and various types of cancers and to provide new options for treatment to address issues of tolerance of proliferating cells to the existing chemotherapeutic agents and treatment regimes. Agents which can act synergistically with other chemotherapeutics are highly sought after.

Prostate cancer is an increasing problem amongst men, particularly in Western countries. With the exception of lung cancer, prostate cancer is the cancer which results in the greatest mortality in Australian men. The symptoms of prostate cancer and related problems include difficult, painful and frequent urination, blood in the urine, and pain in the lower back, hips and upper thighs, and painful ejaculation.

Testosterone is an androgen which is reductively converted to dihydrotestosterone (DHT) by the enzyme 5-α-reductase. DHT is 40 times more potent as a growth factor at the androgen receptor than testosterone. Prostatic cancer is initially dependent on androgen for growth and division. Slowing the growth of androgen dependant prostate cancer is therefore desirable by inhibiting the conversion of testosterone to DHT.

The first effective systematic therapy for prostate cancer, was castration in conjunction with oral estrogen (a form of androgen ablation) in the 1940s. Remarkably androgen ablation remains the most frequently used therapy for prostate cancer, even today. Currently however, androgen ablation is not achieved through estrogen treatment, but most commonly by the use of the steroidal Finasteride and more recently with Dutasteride. Problems with these drugs include potential steroidal-related side effects.

Once prostate cancer becomes hormone independent, androgen ablation techniques are of little use, and then the usual next line of defence is the use of cytotoxic agents. Monotherapy for prostate cancer will almost inevitably lead to drug resistance and so a way to circumvent this problem is the use of multiple drugs for treatment. Multiple drug therapy commonly alleviates pain associated with cancer as well as decreasing the dosage of some toxic drugs.

Accordingly, there is also a need for access to additional and complimentary compounds useful for the amelioration and treatment of symptoms associated with prostate enlargement and related cellular proliferation and cancers including compounds possibly active as 5-α-reductase inhibitors and/or α_{1A} adrenoceptor antagonists.

Whilst not wishing to be bound by theory the compounds of the present invention are thought to regulate a wide variety of signal transduction processes within animal cells and that these signal transduction processes are involved in a wide range of functions that are vital to the survival and function of all animal cells. Therefore, these compounds have broad-ranging and important health benefits in animals including humans, and in particular have the potential to prevent and treat important and common human diseases, disorders and functions, which represent a substantial unexpected benefit.

The invention is further illustrated by the following non-limiting Examples.

### EXAMPLES

### 1.0 Synthesis

### Nucleophilic Additions to Isoflavylium Salts

2-Substituted isoflavonoid compounds of the subject invention are available by nucleophilic additions to isoflavylium salts.

### Example 1

### 2-Allyl-4′,7-diacetoxy-isoflav-3-ene

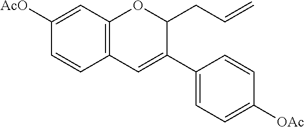

4′,7-Diacetoxy-isoflav-3-ene (1.05 g, 3.24 mmol) and tritylium hexafluorophosphate (1.46 g, 3.76 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Allyltributyltin (2 ml, 6.45 mmol) was added to the stirring suspension under an atmosphere of nitrogen. The reaction mixture was stirred at room temperature for 17 hours, after which the volume was concentrated in vacuo. The pale yellow oil thus obtained was passed through a plug of silica with dichloromethane. The solvent was evaporated under reduced pressure to give a white solid, which was recrystallised from methanol to give the title compound as long, white needles (yield 270 mg, 23%).

¹H NMR (400 MHz, CDCl₃) δ 7.47 (2H, d, J=8.7 Hz, H2′,6′), 7.12 (2H, d, J=8.7 Hz, H3′,5′), 7.07 (1H, d, J=8.1 Hz, H5), 6.72 (1H, br s, H4), 6.67 (1H, dd, J=2.2 Hz, 8.1 Hz, H6), 6.63 (1H, d, J=2.2 Hz, H8), 5.92-5.81 (1H, m, H2″), 5.35 (1H, dd, J=3.3 Hz, 9.1 Hz, H2), 5.09-5.01 (2H, m, H3″a,b), 2.63-2.53 (1H, m, H1″a), 2.34-2.25 (7H, m, 1″b, acetate CH₃).

### Example 2

### 2-Allyl-4′,7-dihydroxy-isoflav-3-ene

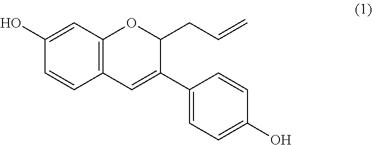

2-Allyl-4′,7-diacetoxy-isoflav-3-ene (113 mg, 0.31 mmol) was suspended in methanol (ca. 5 ml). Potassium hydroxide solution (0.6 ml, 0.6 mmol, 1M in H₂O) was added. The reaction mixture was stirred at room temperature for one hour before being neutralised (pH 6-7) with acetic acid (ca. 0.5 ml, 1M in H₂O). The neutralised reaction mixture was diluted with water (ca. 15 ml) and stirred at room temperature for 18 hours. The light brown precipitate that formed during this time was collected via vacuum filtration to give the title compound (yield 43 mg, 49%).

¹H NMR (400 MHz, d₆-DMSO) δ 9.53 (2H, br s, OH), 7.37 (2H, d, J=8.4 Hz, H2′,6′), 6.95 (1H, d, J=8.1 Hz, H5), 6.77 (2H, d, J=8.4 Hz, H3′,5′), 6.74 (1H, br s, H4), 6.32 (1H, dd, J=2.2 Hz, 8.1 Hz, H6), 6.22 (1H, d, J=2.2 Hz, H8), 5.92-5.80 (1H, m, H2″), 5.34 (1H, dd, J=2.9 Hz, 9.1 Hz, H2), 5.04-4.97 (2H, m, 3″a,b), 2.44-2.34 (1H, m, ra), 2.21-2.13 (1H, m, 1″b).

### Example 3

### 4′,7-Diacetoxy-2-ethyl-isoflav-3-ene

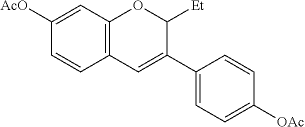

4′,7-Diacetoxy-isoflav-3-ene (1.00 g, 3.08 mmol) and tritylium hexafluorophosphate (1.43 g, 3.69 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Diethylzinc solution (4.0 ml, 4.0 mmol, 1M in hexane) was added to the stirring suspension under an atmosphere of nitrogen. The reaction mixture was stirred at room temperature for 40 minutes before being quenched with saturated aqueous ammonium chloride (ca. 100 ml). The dichloromethane layer was collected, washed with water (2×50 ml) and brine (ca. 50 ml) and dried over MgSO₄. Solvent was removed in vacuo to afford a yellow solid, which was recrystallised from ethyl acetate to give the title compound as off-white needles (yield 300 mg, 26%).

¹H NMR (400 MHz, CDCl₃) δ 7.46 (2H, d, J=8.8 Hz, H2′,6′), 7.11 (2H, d, J=8.8 Hz, H3′,5′), 7.06 (1H, d, J=8.8 Hz, H5), 6.68 (1H, br s, H4), 6.66-6.63 (2H, m, H6, 8), 5.20 (1H, dd, J=3.3 Hz, 9.5 Hz, H2), 2.31 (3H, s, acetate CH₃), 2.27 (3H, s, acetate CH₃), 1.86-1.79 (1H, m, H1″a), 1.61-1.54 (1H, m, H1″b), 1.00 (3H, t, J=7.3 Hz, H2″).

### Example 4

### 4′,7-Dihydroxy-2-ethyl-isoflav-3-ene

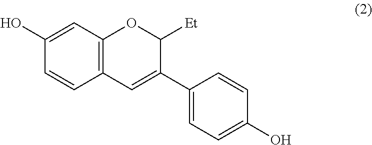

4′,7-Diacetoxy-2-ethyl-isoflav-3-ene (48 mg, 0.13 mmol) was suspended in methanol (ca. 2 ml). Potassium hydroxide solution (0.5 ml, 0.5 mmol, 1M in H₂O) was added. The reaction mixture was stirred at room temperature for two hours before being neutralised (pH 6-7) with acetic acid (ca. 0.5 ml, 1M in H₂O). The neutralised reaction mixture was diluted with water (ca. 15 ml) and stirred at room temperature for 18 hours. The light brown precipitate that formed during this time was collected via vacuum filtration to give the title compound (yield 17 mg, 47%).

¹H NMR (400 MHz, d₆-DMSO) δ 9.50 (2H, br s, OH), 7.34 (2H, d, J=8.8 Hz, H2′,6′), 6.91 (1H, d, J=8.4 Hz, H5), 6.74 (2H, d J=8.8 Hz, H 3′,5′), 6.68 (1H, br s, H4), 6.28 (1H, dd, J=2.2 Hz, 8.4 Hz, H6), 6.23 (1H, d, J=2.2 Hz, H8), 5.15 (1H dd, J=3.3 Hz, 9.5 Hz, H5), 1.66-1.54 (1H, m, H1″a), 1.46-1.34 (1H, m, H1″b), 0.90 (3H, t, J=7.3 Hz, H2″).

### Example 5

### 4′,7-Diacetoxy-2-ethyl-isoflavan

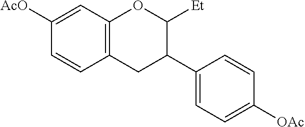

4′,7-Diacetoxy-2-ethyl-isoflav-3-ene (120 mg, 0.34 mmol) and palladium on alumina (450 mg, 10% wt/wt) were suspended in absolute ethanol (10 ml). The mixture was stirred under hydrogen (1 bar) for 90 minutes. The palladium catalyst was removed from the reaction mixture via filtration through a plug of Celite. The filtrate was reduced in vacuo to give the title compound as a pale yellow solid (yield 103 mg, 85%).

¹H NMR (400 MHz, CDCl₃) δ 7.17 (2H, d, J=8.4 Hz, H2′,6′), 7.07 (1H, d, J=9.1 Hz, H5), 6.98 (2H, d, J=8.4 Hz, H3′,5′), 6.65-6.62 (2H, m, H6, 8), 4.18-4.12 (1H, m, H2), 3.33-3.27 (1H, m, H3), 3.07 (2H, ddd, J=6.6 Hz, 16.8 Hz, 71.7 Hz, H4), 2.29 (3H, s, acetate CH₃), 2.28 (3H, s, acetate CH₃), 1.54-1.45 (1H, m, 1″a), 1.40-1.32 (1H, m, 1″b), 0.96 (3H, t, J=7.3 Hz).

### Example 6

### 4′,7-Dihydroxy-2-ethyl-isoflavan

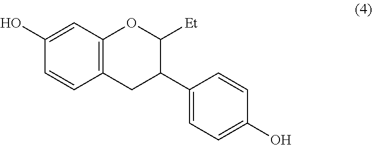

4′,7-Diacetoxy-2-ethyl-isoflavan (55 mg, 0.16 mmol) was suspended in methanol (ca. 3 ml). Potassium hydroxide solution (0.4 ml, 0.4 mmol, 1M in H₂O) was added. The reaction mixture was stirred at room temperature for one hour before being neutralised (pH 6-7) with acetic acid (ca. 0.5 ml, 1M in H₂O). The neutralised reaction mixture was diluted with water (ca. 15 ml) and stirred at room temperature for 3 days. The brown precipitate that formed during this time was collected via vacuum filtration to give the title compound (yield 17 mg, 40%)

¹H NMR (400 MHz, d₆-DMSO) δ 9.18 (2H, br s, OH), 6.94 (2H, d, J=8.4 Hz, H2′,6′), 6.83 (1H, d, J=8.1 Hz, H5), 6.61 (2H, d, J=8.4 Hz, H3′,5′), 6.26 (1H, dd, J=2.6 Hz, 8.1 Hz, H6), 6.16 (1H, d, J=2.6 Hz, H8), 4.10-4.00 (1H, m, H2), 3.14-3.09 (1H, m, H3), 2.84 (2H, ddd, J=6.6 Hz, 16.1 Hz, 94.4 Hz, H4), 1.39-1.16 (2H, m, H1″), 0.85 (3H, t, J=7.3 Hz, H2″).

### Example 7

### 4′,7-Diacetoxy-2-methyl-isoflav-3-ene

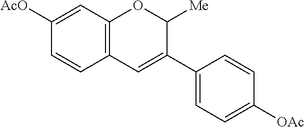

4′,7-Diacetoxy-isoflav-3-ene (1.00 g, 3.08 mmol) and tritylium hexafluorophosphate (1.42 g, 3.66 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Dimethylzinc solution (4.0 ml, 4.0 mmol, 1M in heptane) was added to the stirring suspension under an atmosphere of nitrogen. The reaction mixture was stirred at room temperature for one hour before being quenched with saturated aqueous ammonium chloride (ca. 100 ml). The dichloromethane layer was collected, washed with water (2×50 ml) and brine (ca. 50 ml) and dried over MgSO₄. Solvent was removed in vacuo to afford the title compound as a pale green solid (yield 740 mg, 71%).

¹H NMR (400 MHz, CDCl₃) δ 7.47 (2H, d, J=8.8 Hz, H2′,6′), 7.11 (2H, d, J=8.8 Hz, H3′,5′), 7.07 (1H, d, J=8.1 Hz, H5), 6.69 (1H, br s, H4), 6.66 (1H, dd, J=2.2 Hz, 8.1 Hz, H6), 6.63 (1H, d, J=2.2 Hz, H8), 5.45 (1H, q, J=6.6 Hz, H2), 2.31 (3H, s, acetate CH₃), 2.29 (3H, s, acetate CH₃), 1.39 (3H, d, J=6.6 Hz, 2-CH₃).

### Example 8

### 4′,7-Dihydroxy-2-methyl-isoflav-3-ene

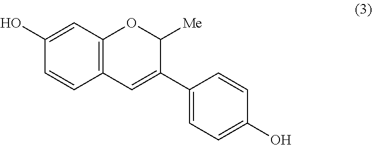

4′,7-Diacetoxy-2-methyl-isoflav-3-ene (122 mg, 0.36 mmol) was suspended in methanol (ca. 5 ml). Potassium hydroxide solution (0.7 ml, 0.7 mmol, 1M in H₂O) was added. The reaction mixture was stirred at room temperature for two hours before being neutralised (pH 6-7) with acetic acid (ca. 0.5 ml, 1M in H₂O). The neutralised reaction mixture was diluted with water (ca. 15 ml) and stirred at room temperature for 18 hours. The pale green precipitate that formed during this time was collected via vacuum filtration to give the title compound (yield 62 mg, 68%).

¹H NMR (400 MHz, d₆-DMSO) δ 9.53 (2H, br s, OH), 7.37 (2H, d, J=8.8 Hz, H2′,6′), 6.95 (1H, d, J=8.4 Hz, H5), 6.77 (2H, d, J=8.8 Hz, H3′,5′), 6.70 (1H, br s, H4), 6.31 (1H, dd, J=2.2 Hz, 8.4 Hz, H6), 6.24 (1H, d, J=2.2 Hz, H8), 5.43 (1H, q, J=6.6 Hz, H2), 1.23 (3H, d, J=6.6 Hz, 2-CH₃).

### Example 9

### 4′,7-Diacetoxy-2-methyl-isoflavan

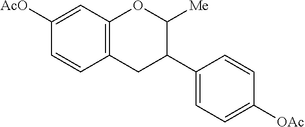

4′,7-Diacetoxy-2-methyl-isoflav-3-ene (160 mg, 0.47 mmol) and palladium on alumina (480 mg, 10% wt/wt) were suspended in absolute ethanol (10 ml). The mixture was stirred under hydrogen (1 bar) for 90 minutes. The palladium catalyst was removed from the reaction mixture via filtration through a plug of Celite. The filtrate was reduced in vacuo to give the title compound as a pale green solid (yield 136 mg, 84%).

¹H NMR (400 MHz, CDCl₃) δ 7.18 (2H, d, J=8.4 Hz, H2′,6′), 7.08 (1H, d, J=8.1 Hz, H5), 7.00 (2H, d, J=8.4 Hz, H3′,5′), 6.64 (1H, dd, J=2.2 Hz, 8.1 Hz, H6), 6.60 (1H, d, J=2.2 Hz, H8), 4.52-4.45 (1H, m, H2), 3.29-3.24 (1H, m, H3), 3.08 (2H, ddd, J=6.2 Hz, 16.5 Hz, 38.8 Hz, H4), 2.30-2.28 (6H, m, acetate CH₃), 1.13 (3H, d, J=6.6 Hz, 2-CH₃)

### Example 10

### 4′,7-Dihydroxy-2-methyl-isoflavan

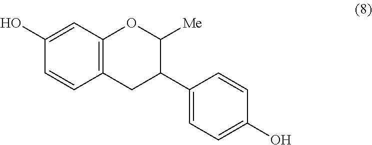

4′,7-Diacetoxy-2-methyl-isoflavan (66 mg, 0.19 mmol) was suspended in methanol (ca. 3 ml). Potassium hydroxide solution (0.5 ml, 0.5 mmol, 1M in H₂O) was added. The reaction mixture was stirred at room temperature for one hour before being neutralised (pH 6-7) with acetic acid (ca. 0.5 ml, 1M in H₂O). The neutralised reaction mixture was diluted with water (ca. 15 ml) and stirred at room temperature for 3 days. The reddish-brown precipitate that formed during this time was collected via vacuum filtration to give the title compound (yield 16 mg, 32%)

¹H NMR (400 MHz, d₆-DMSO) δ 9.22 (2H, br s, OH), 6.97 (2H, d, J=8.4 Hz, H2′,6′), 6.87 (1H, d, J=8.1 Hz, H5), 6.65 (2H, d, J=8.4 Hz, H3′,5′), 6.29 (1H, dd, J=2.2 Hz, 8.1 Hz, H6), 6.17 (1H, d, J=2.2 Hz, H8), 4.14-4-03 (1H, m, H2), 3.11-3.06 (1H, m H3), 2.87 (2H, ddd, J=6.2 Hz, 16.1 Hz, 60.0 Hz, H4), 0.99 (3H, d, J=6.6 Hz, 2-CH₃).

### Example 11

### 4′,7-Diacetoxy-2,8-dimethyl-isoflav-3-ene

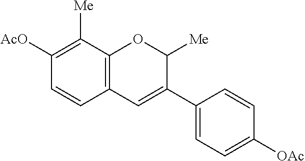

4′,7-Diacetoxy-8-methyl-isoflav-3-ene (1.07 g, 3.16 mmol) and tritylium hexafluorophosphate (1.33 g, 3.43 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Dimethylzinc solution (4.0 ml, 4.0 mmol, 1M in heptane) was added to the stirring suspension under an atmosphere of nitrogen. The reaction mixture was stirred at room temperature for 90 minutes before being quenched with saturated aqueous ammonium chloride (ca. 100 ml). The dichloromethane layer was collected, washed with water (2×50 ml) and brine (ca. 50 ml) and dried over MgSO₄. Solvent was removed in vacuo to afford a green/brown oil, which was recrystallised from methanol to give the title compound as green needles (yield 238 mg, 21%).

¹H NMR (400 MHz, CDCl₃) δ 7.47 (2H, d, J=8.8 Hz, H2′,6′), 7.11 (2H, d, J=8.8 Hz, H3′,5′), 6.94 (1H, d, J=8.1 Hz, H5), 6.69 (1H, br s, H4), 6.61 (1H, d, J=8.1 Hz, H6), 5.51 (1H, q, J=6.6 Hz, H2), 2.32 (3H, s, acetate CH₃), 2.31 (3H, s, acetate CH₃), 2.05 (3H, s, 8-CH₃), 1.38 (3H, d, J=6.6 Hz, 2-CH₃).

### Example 12

### 4′,7-Dihydroxy-2,8-dimethyl-isoflav-3-ene

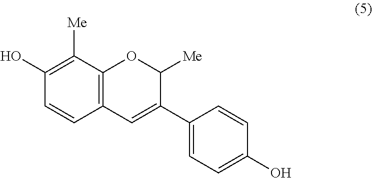

4′,7-Diacetoxy-2,8-dimethyl-isoflav-3-ene (67 mg, 0.19 mmol) was suspended in methanol (ca. 3 ml). Potassium hydroxide solution (0.4 ml, 0.4 mmol, 1M in H₂O) was added. The reaction mixture was stirred at room temperature for one hour before being neutralised (pH 6-7) with acetic acid (ca. 0.5 ml, 1M in H₂O). The neutralised reaction mixture was diluted with water (ca. 15 ml) and extracted with ethyl acetate (3×10 ml). The combined extracts were washed with brine (ca. 25 ml) and dried over MgSO₄. Solvent was evaporated in vacuo to give the title compound as a reddish-brown solid (yield 40 mg, 78%)

¹H NMR (400 MHz, d₆-DMSO) δ 9.57 (1H, br s, OH), 9.41 (1H, br s, OH), 7.37 (2H, d, J=8.8 Hz, H2′,6′), 6.81-6.72 (3H, m, H5, 3′,5′), 6.70 (1H, br s, H4), 6.38 (1H, d, J=8.1 Hz, H6), 5.50 (1H, q, J=6.2 Hz, H2), 1.97 (3H, s, 8-CH₃), 1.21 (3H, d, J=6.2 Hz, 2-CH₃).

### Example 13

### 3′,7-Diacetoxy-2-methyl-isoflav-3-ene

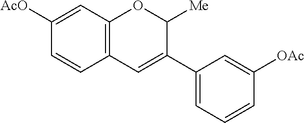

3′,7-Diacetoxy-isoflav-3-ene (1.10 g, 3.39 mmol) and tritylium hexafluorophosphate (1.45 g, 3.74 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Dimethylzinc solution (4.0 ml, 4.0 mmol, 1M in heptane) was added to the stirring suspension under an atmosphere of nitrogen. The reaction mixture was stirred at room temperature for one hour before being quenched with saturated aqueous ammonium chloride (ca. 100 ml). The dichloromethane layer was collected, washed with water (2×50 ml) and brine (ca. 50 ml) and dried over MgSO₄. Solvent was removed in vacuo to give a green oil, which was recrystallised from methanol to afford the title compound as fine, white crystals. The recrystallisation filtrate was reduced in vacuo to afford a second crop of the title compound as a green oil (combined yield 287 mg, 25%).

¹H NMR (400 MHz, CDCl₃) δ 7.39 (1H, t, J=8.1 Hz, H5′), 7.31 (1H, dt, J=1.1 Hz, 8.1 Hz, H6′), 7.19 (1H, dd, J=1.1 Hz, 2.2 Hz, H2′), 7.07 (1H, d, J=8.1 Hz, H5), 7.04 (1H, ddd, J=1.1 Hz, 2.2 Hz, 8.1 Hz, H4′), 6.73 (1H, br s, H4), 6.66 (1H, dd, J=2.6 Hz, 8.1 Hz, H6), 6.63 (1H, d, J=2.6 Hz, H8), 5.44 (1H, q, J=6.6 Hz, H2), 2.32 (3H, s, acetate CH₃), 2.28 (3H, s, acetate CH₃), 1.39 (3H, d, J=6.6 Hz, 2-CH₃).

### Example 14

### 3′,7-Dihydroxy-2-methyl-isoflav-3-ene

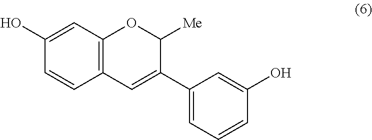

3′,7-Diacetoxy-2-methyl-isoflav-3-ene (105 mg, 0.31 mmol) was suspended in methanol (ca. 5 ml). Potassium hydroxide solution (0.6 ml, 0.6 mmol, 1M in H₂O) was added. The reaction mixture was stirred at room temperature for two hours before being neutralised (pH 6-7) with acetic acid (ca. 0.5 ml, 1M in H₂O). The neutralised reaction mixture was diluted with water (ca. 15 ml) and extracted with ethyl acetate (3×10 ml). The combined extracts were washed with brine (ca. 25 ml) and dried over MgSO₄. Solvent was evaporated in vacuo to give the title compound as a brownish-green solid (yield 53 mg, 67%).

¹H NMR (400 MHz, d₆-DMSO) δ 9.61 (1H, br s, OH), 9.45 (1H, br s, OH), 7.14 (1H, t, J=8.1 Hz, H5′), 6.98 (1H, d, J=8.1 Hz, H5), 6.94 (1H, dt, J=0.7 Hz, 8.1 Hz, H6′), 6.87 (1H, dd, J=0.7 Hz, 2.2 Hz, HT), 6.79 (1H, br s, H4), 6.66 (1H, ddd, J=0.7 Hz, 2.2 Hz, 8.1 Hz, H4′), 6.31 (1H, dd, J=2.2 Hz, 8.1 Hz, H6), 6.23 (1H, d, J=2.2 Hz, H8), 5.39 (1H, q, J=6.66 Hz, H2), 1.22 (3H, d, J=6.6 Hz, 2-CH₃).

### Example 15

### 4′,7-Diacetoxy-2,5-dimethyl-isoflav-3-ene

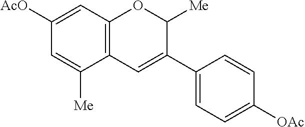

4′,7-Diacetoxy-5-methyl-isoflav-3-ene (510 mg, 1.51 mmol) and tritylium hexafluorophosphate (670 mg, 1.73 mmol) were dissolved in dry dichloromethane (50 ml). The reaction mixture was stirred at room temperature, under nitrogen, for one hour. The yellow solid that precipitated during this time was isolated via vacuum filtration and resuspended in dry dichloromethane (50 ml). Dimethylzinc solution (2.0 ml, 2.0 mmol, 1M in heptane) was added to the stirring suspension under an atmosphere of nitrogen. The reaction mixture was stirred at room temperature for two hours before being quenched with saturated aqueous ammonium chloride (ca. 100 ml). The dichloromethane layer was collected, washed with water (2×50 ml) and brine (ca. 50 ml) and dried over MgSO₄. Solvent was removed in vacuo to afford a pale green oil, which was recrystallised from methanol to give the title compound as brownish-green needles (yield 138 mg, 26%).

¹H NMR (400 MHz, CDCl₃) δ 7.48 (2H, d, J=8.8 Hz, H2′,6′), 7.12 (2H, d, J=8.8 Hz, H3′,5′), 6.82 (1H, br s, 4H), 6.52 (1H, d, J=2.2 Hz, H6), 6.50 (1H, d, J=2.2 Hz, H8), 5.41 (1H, q, J=6.6 Hz, H2), 2.36 (3H, s, 5-CH₃), 2.32 (3H, s, acetate CH₃), 2.27 (3H, s, acetate CH₃), 1.38 (3H, d, J=6.6 Hz, 2-CH₃).

### Example 16

### 4′,7-Dihydroxy-2,5-dimethyl-isoflav-3-ene

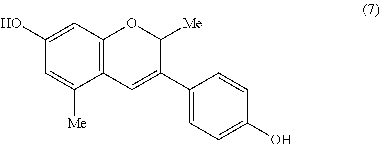

4′,7-Diacetoxy-2,5-dimethyl-isoflav-3-ene (111 mg, 0.31 mmol) was suspended in methanol (ca. 6 ml). Potassium hydroxide solution (0.5 ml, 0.5 mmol, 1M in H₂O) was added. The reaction mixture was stirred at room temperature for 90 minutes before being neutralised (pH 6-7) with acetic acid (ca. 0.5 ml, 1M in H₂O). The neutralised reaction mixture was diluted with water (ca. 15 ml) and extracted with ethyl acetate (3×10 ml). The combined extracts were washed with brine (ca. 25 ml) and dried over MgSO₄. Solvent was evaporated in vacuo to give the title compound as a reddish-brown solid (yield 45 mg, 53%).

¹H NMR (400 MHz, d₆-DMSO) δ 9.56 (1H, br s, OH), 9.41 (1H, br s, OH), 7.40 (2H, d, J=8.4 Hz, H2′,6′), 6.79-6.76 (3H, m, H4, 3′,5′), 6.19 (1H, d, J=2.2 Hz, H6), 6.10 (1H, d, J=2.2 Hz, H8), 5.38 (1H, q, J=6.6 Hz, H2), 2.25 (3H, s, 5-CH₃), 1.22 (3H, d, J=6.6 Hz, 2-CH₃).

### Example 17

### 4′,7-Diacetoxy-2-cyano-isoflav-3-ene

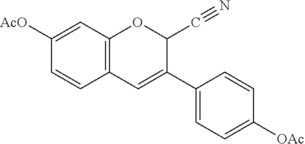

Freshly distilled anhydrous DCM (50 mL) was added to 4′,7-Diacetoxy-isoflav-3-ene (0.503 mg, 1.550 mmol), trityl hexafluorophosphate (0.879 g, 2.265 mmol) and powdered 3 Å molecular sieves. The murky brown solution was stirred at room temperature for 30 min. Trimethylsilyl cyanide (0.480 g, 485 mmol) was then injected into the reaction mixture and left to stir at rt overnight. The solution was filtered. The filtrate was then dried and dissolved in DCM (4 mL) and applied to a silica column. A gradient column was then run starting with 5% hexane in DCM then run using 100% DCM. The product 108a was collected in 80% yield as a white solid (0.431 g, 1.235 mmol, mp 156-158° C.).

¹H-NMR (500 MHz, CDCl₃): δ 7.49 (d, 2H, J≦8.7, H-2′/6′), 7.23 (d, 1H, J=8.0, H-5), 7.18 (d, 2H, J=8.6, H-3′/5′), 6.97 (s, 1H, H-4), 6.85 (d of d, 1H, J=2.5, 8.1, H-6), 6.84 (s, 1H, H-8), 6.01 (s, 1H, H-2), 2.32 (s, 3H, CH₃), 2.30 (s, 3H, CH₃). ¹³C-NMR (75 MHz, CDCl₃): 169.1 (C═O), 168.9 (C═O), 151.9 (C8a′), 151.1 (C7), 150.3 (C4′), 131.9 (C3), 128.3 (C5), 126.2 (C2′), 125.9 (C1′), 122.4 (C3′), 122.3 (C4), 119.1 (C4a), 117.0 (C6), 116.0 (CN), 110.5 (C8), 64.3 (C2), 21.1 (CH₃). MS (CI⁺): m/z 323 (—CN, 100%), 349 (M⁺, 8%). MS (ES⁺): m/z 367 (M⁺+H₂O, 100%). Microanalysis: Found: C=69.20%; H=4.34, 4.35%; N=3.67%; C₂₀H₁₅NO₅ requires: C=68.76%; H=4.33%, N=4.01%.

### Example 18

### 2-Cyano-4′,7-hydroxyisoflav-3-ene

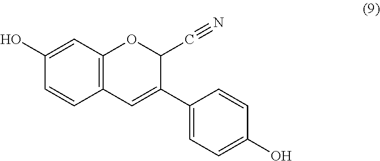

The diacetoxy nitrile from Example 17 above (0.0845 g, 0.243 mmol) was stirred at room temperature in THF (3 mL) and 50% methanol/water 1M NaOH (9 mL) for 4 h. The solution was then neutralized with 5M HCl and extracted with DCM (2×50 mL). The DCM extract was the concentrated, and then applied to a silica plug (short column). 15% Diethyl ether in DCM was passed through the plug to give the title nitrile as a red glass (0.061 g, 0.233 mmol) in 96% yield.

¹H-NMR (300 MHz, d₆-acetone): δ 8.79 (bs, H, OH), 7.52 (d, 2H, J=8.7, H-2′), 7.19 (d, 1H, J=8.1, H-5), 7.09 (s, 1H, H-4), 6.93 (d, 2H, J=8.7, H-3′), 6.59 (d of d, 1H, J=8.4, 2.4, H-6), 6.55 (d, of d, J=2.1, 0.3, H-8), 6.46 (s, 1H, H-2). ¹³C-NMR (75 MHz, d₆-acetone): 160.0 (C8a), 158.1 (C7), 156.6 (C4), 142.8 (C3), 139.7 (CP), 128.9 (CT), 125.8 (C5), 115.4 (C3′), 113.4 (C4), 110.3 (CN), 105.0 (C6), 103.5 (C4a), 102.8 (C8), 61.2 (C2). MS (CI⁺): m/z 266 (M+1, 3%), 239 (isoflavylium, 100%). Microanalysis: Found: C=72.43%; H=4.21, N=5.25%; C₂₁H₂₀O₆ requires: C=72.45%; H=4.18%, N=5.28%.

### Example 19

### 4′,7-Diacetoxy-2-(2-thiazoyl)-isoflav-3-ene

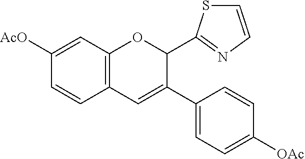

Anhydrous DCM (50 mL) was added to 4′,7-Diacetoxy-isoflav-3-ene (0.451 g, 1.390 mmol), trityl hexafluorophosphate (0.849 g, 2.188 mmol) and powdered 3 Å molecular sieves. The murky brown solution was stirred at room temperature for 30 min. 2-(Trimethylsilyl)thiazole (0.4025 g, 2.564 mmol) was then injected into the reaction mixture and the mixture left to stir at room temperature for 2 h. The solution was filtered. The filtrate was then dried (MgSO₄) and dissolved in DCM (4 mL) and applied to a silica column. A gradient column was then run starting with 100% DCM increasing polarity by 5% ethyl acetate increments and ending with 10% ethyl acetate in DCM. The product was collected in 78% yield as a creamy white solid (0.695 g 1.707 mmol, 136-138 mp).

¹H-NMR (300 MHz, CDCl₃): δ 7.60 (bd, 1H, J=2.7, H-2″), 7.37 (d, 2H, J=8.7, H-2′/6′), 7.23 (d, 1H, J=6.3, H-5), 7.08 (d, 1H, J=2.7, H-3″), 6.93 (d, 2H, J=8.4, H-375′), 6.89 (s, 1H, H-4), 6.57 (d of d, 1H, J=8.4, 2.7, H-6), 6.53 (d, 1H, J=2.4, H-8), 6.44 (bs, 1H, H-2), 2.13 (s, 3H, CH₃), 2.10 (s, 3H, CH₃). ¹³C-NMR (75 MHz, CDCl₃): δ 169.5 (C═O), 169.3 (C═O), 169.3 (C1″), 151.8 (C8a), 151.7 (C7), 150.8 (C4′) 143.3 (C4″), 134.0 (C3), 131.5 (C1′), 127.9 (C5), 126.9 (CT), 122.2 (C3′), 121.1 (C3″), 120.9 (C4), 120.2 (C4a), 115.7 (C6), 110.6 (C8), 74.7 (C2), 21.3 (CH₃CO). MS (CI⁺): m/z 407.8 (M+1, 100%). HR (CI⁺) MS: m/z calcd for [M⁺] C₂₂H₁₇NO₅S: 408.0906, found: 408.0887.

### Example 20

### 4′,7-Dihydroxy-2-(2-thiazoyl)-isoflav-3-ene

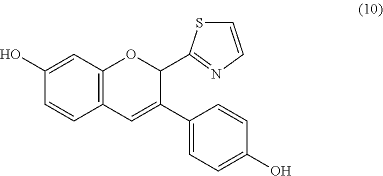

The diacetoxy compound of Example 19 was deprotected according to the general method of Example 18 to afford the title compound.

### Example 21

### 4′,7-Diacetoxy-2-ethynyl-isoflav-3-ene

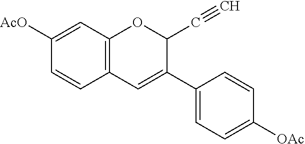

Anhydrous DCM (50 mL) was added to 4′,7-diacetoxy-isoflav-3-ene (0.646 g, 1.995 mmol), trityl hexafluorophosphate (1.159 g, 2.987 mmol) and powdered 3 Å molecular sieves. The brown-yellow solution was stirred at room temperature for 1 h. TMS acetylene (1.4 mL) was then injected into the reaction mixture and the mixture left to stir at room temperature for 1 h. The solution was filtered. The filtrate was then dried (MgSO₄) and dissolved in DCM (4 mL) and applied to a silica column. A gradient column was then run starting with 100% DCM increasing polarity by 5% ethyl acetate increments and ending with 10% ethyl acetate in DCM. The product was collected in 7% yield as a creamy white solid (0.047 g, 0.1397 mmol, mp 179-181° C. decomp.).

¹H-NMR (300 MHz, CDCl₃): δ 7.56 (d, 2H, J=8.7, H-2′), 7.19 (d, 1H, J=8.1, H-5), 7.10 (d, 2H, J=8.7, H-3′), 6.91 (s, 1H, H-4), 6.80 (d, 1H, J=2.4, H-8), 6.75 (d of d, 1H, J=8.4, 2.4, H-6), 6.19 (d, 1H, J=7.5, H-2), 3.71 (d, 1H, J=7.8, C≡CH), 2.30 (s, 3H, COCH₃), 2.29 (s, 3H, COCH₃). ¹³C-NMR (75 MHz, CDCl₃): δ 169.8 (C═O), 169.7 (C═O), 134.2 (C7), 131.0 (C8a), 128.0 (C4′), 127.0 (C3), 127.0 (Cr), 122.2 (C5), 122.1 (C2′), 120.8 (C4), 119.0 (C3′), 115.6 (C6), 110.8 (C8), 91.7 (C2), 21.4 (COCH₃). MS (CI⁺): m/z 323 (isoflavylium, M-C≡CH, 100%). Microanalysis: Found: C=72.39%; H=4.66%; C₂₀H₁₆O₅ requires: C=72.41%; H=4.63%.

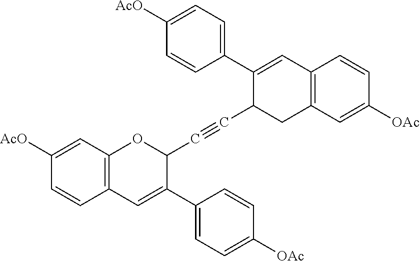

A second, dimeric product was produced in 67% yield (0.4478 g, 0.6683 mmol, mp 237-238° C. decomp.).

¹H-NMR (300 MHz, d-DMF): δ 7.11 (d, 2H, J=9.0, H-2′), 7.02 (d, 1H, J=8.4, H-5), 6.98 (s, 1H, H-4), 6.73 (d of d, 1H, J=2.4, 0.3, H-8), 6.66 (d, 2H, J=9.0, H-3′), 6.53 (s, 1H, H-2), 6.51 (d of d, 1H, J=8.4, 2.4, H-6), 1.94 (s, 3H, COCH₃), 1.88 (s, 3H, COCH₃). ¹³C-NMR (75 MHz, d6-DMSO): δ 169.2 (C═O), 169.1 (C═O), 151.8 (C7), 150.8 (C8a), 150.2 (C4″), 133.0 (C3), 128.4 (C1′), 128.2 (C5), 126.4 (CT), 122.2 (C4), 121.5 (C3′), 119.4 (C6), 116.3 (C4a), 110.7 (C8), 92.5 (C═), 91.7 (C2), 20.4 (COCH₃), 20.3 (COCH₃). MS (ES⁺): m/z 323 (M+1, 100%). Microanalysis: Found: C=69.27%, H=4.62%; C₄₁H₃₄O₁₀ requires: C=69.28%, H=4.60%,

### Example 22

### 2-Ethynyl-4′,7-dihydroxyisoflav-3-ene

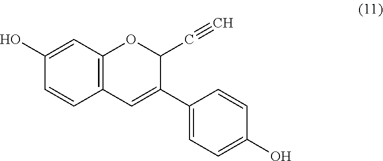

The monomeric diacetoxy compound of Example 21 was deprotected according to the general method of Example 18 to afford the title compound.

### Example 23

### 4′,7-Diacetoxy-2-(N—(BOC)aminomethyl)-isoflav-3-ene

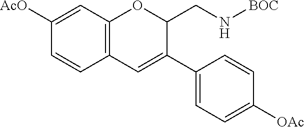

Anhydrous DCM (50 mL) was added to 4′,7-Diacetoxy-isoflav-3-ene (0.404 g, 1.246 mmol), trityl hexafluorophosphate (0.612 g, 1.576 mmol) and powdered 3 Å molecular sieves. The orange-yellow solution was stirred at rt for 30 min. Anhydrous t-butyl N-(t-butyloxycarbonyl)-N-(trimethylysilyl)methyl carbamate (prepared by BOC protection of trimethylsilylmethylamine) was then injected into the reaction mixture and the mixture stirred at room temperature overnight. The solution was filtered. The filtrate was then dried (MgSO₄) and dissolved in DCM (4 mL) and applied to a silica column. A gradient column was then run starting with 100% DCM increasing polarity by 5% ethyl acetate increments and ending with 10% ethyl acetate in DCM. The product was collected in 45% yield as a creamy white solid (0.255 g, 0.5612 mmol, mp 52-54° C.).

¹H-NMR (300 MHz, CDCl₃): δ 7.64 (d, 1H, J=8.7, H-5), 7.52 (d, 1H, j=8.7, H-2′), 7.48 (d, 1H, J=8.7, H-2′), 7.13 (d, 2H, H=8.7), 6.98 (d of d, 1H, J=8.7, 2.4, H-6), 6.91 (d, 1H, J=2.8, H-4), 6.67 (d, 0.6H, J=2.1, H-8), 6.66 (d, 0.4H, J=2.1, H-8), 5.44 (d of d, 0.4H, J=11.0, 2.4, NH), 5.15 (d of d of d, 1H, J=11.1, 6.0, 2.4, H-2), 4.86 (bt, 0.3H, J=1.5, NH), 4.76 (bd, 0.3H, J=1.2, NH), 2.59 (d of d, 1H, J=15.0, 10.0, CH₂), 2.20 (d, 1H, J=15.0, CH₂), 2.31 (s, 3H, COCH₃), 2.23 (s, 3H, COCH₃), 2.16 (s 9H,C(CH₃)₃). ¹³C-NMR (75 MHz, CDCl₃): δ 169.5 (COCH₃), 155.2 (CONH), 150.1 (C8a), 147.0 (C7), 143.7 (C4′), 134.7 (C3), 130.7 (C1′), 127.5 (C5), 125.2 (C2′), 122.2 (C2′), 121.9 (C4), 120.2 (C3′), 116.1 (C6), 114.9 (C4a), 109.5 (C8), 75.5 (C(CH₃)), 72.4 (C2), 43.1 (CH₂), 30.7 (C(CH₃)₃), 21.3 (COCH₃). MS (ES⁺): m/z 453.2 (M⁺, 33.3%), 396.2 (M-C(CH₃)₃, 9.2%), 379.2 (M-O—C(CH₃)₃, 25.3%), 338 (M+1-2×boc, 100%), 323.0 (Isoflavylium, 14.5%). HR (ES⁺) MS: m/z calcd for [M⁺] C₂₅H₂₇NO₇: 454.1876, found: 454.1872.

### Example 24

### 2-aminomethyl-4′,7-dihydroxy-isoflav-3-ene

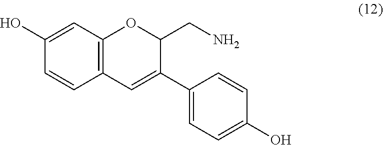

The diacetoxy compound of Example 23 is subjected to reductive removal of the BOC protecting group and acetoxy deprotection according to the general method of Example 18 to afford the title compound.

Amino methyl compound (12) is also obtained following reduction of the diacetoxy nitrile compound of Example 17 with LiAlH₄ and according to the above method.

### Example 25

### 4′,7-Diacetoxy-2-methoxy-isoflav-3-ene

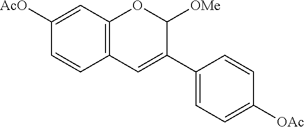

Anhydrous DCM (50 mL) was added to 4′,7-diacetoxy-isoflav-3-ene (0.376 mg, 1.159 mmol), trityl hexafluorophosphate (0.956 g, 2.4647 mmol) and powdered 3 Å molecular sieves. The brown-yellow solution was stirred at room temperature for 30 min. Anhydrous methanol (3 mL) was then injected into the reaction mixture and the mixture stirred at rt overnight. The solution was filtered. The filtrate was then dried (MgSO₄) and dissolved into DCM (4 mL) and applied to a silica column. A gradient column was then run starting with 100% DCM increasing polarity by 5% ethyl acetate increments and ending with 10% ethyl acetate in DCM. The title product was collected in 63% yield as a creamy white solid (0.258 g, 0.731 mmol, mp 149-151° C.).

¹H-NMR (300 MHz, CDCl₃): δ 7.53 (d, 2H, J=9.0, H-2′), 7.24 (d, 1H, J=8.4, H-5), 7.13 (d, 2H, J=8.4, H-3′), 6.98 (s, 1H, H-4), 6.85 (d, 1H, J=2.1, H-8), 6.77 (d of d, 1H, J=8.4, 2.1, H-6), 5.85 (s, 1H, H-2), 3.58 (s, 3H, OCH₃), 2.32 (s, 3H, CH₃CO), 2.30 (s, 3H, CH₃CO). ¹³C-NMR (75 MHz, CDCl₃): δ 169.5 (C═O), 169.3 (C═O), 151.6 (C7), 150.7 (C8a), 150.4 (C4′), 133.7 (C^{a}), 128.3 (C^{a}), 128.1 (C5), 127.0 (C2′), 122.1 (C3′), 121.6 (C4), 119.6 (C4a), 115.6 (C6), 110.5 (C8), 98.3 (C2), 55.5 (OCH₃), 21.3 (CH₃CO).

C^{a}: C3 or C1′

MS (CI⁺): m/z 323 (100%, M-OCH₃). Microanaylsis: Found: C=67.82%; H=5.13, 4.35%; C₂₀H₁₈O₆ requires: C=67.79%; H=5.12%.

### Example 26

### 4′,7-Dihydroxy-2-methoxy-isoflav-3-ene

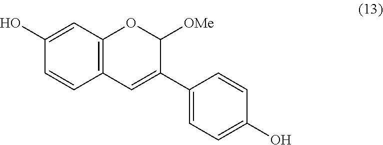

The diacetoxy compound of Example 25 was deprotected according to the general method of Example 18 to afford the title compound.

### Example 27

### 4′,7-Diacetoxy-2-ethoxy-isoflav-3-ene

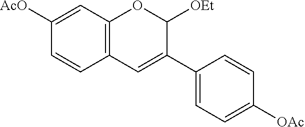

Anhydrous DCM (50 mL) was added to 4′,7-diacetoxy-isoflav-3-ene (0.502 g, 1.549 mmol), trityl hexafluorophosphate (0.872 g, 2.247 mmol) and powdered 3 Å molecular sieves. The murky brown solution was stirred at rt for 30 min. Anhydrous ethanol (3 mL) was then injected into the reaction mixture and left to stir at rt overnight. The solution was filtered. The filtrate was then dried (MgSO₄) and dissolved into DCM (4 mL) and applied to a silica column. A gradient column was then run starting with 100% DCM increasing polarity by 5% ethyl acetate increments and ending with 10% ethyl acetate in DCM. The title product was obtained in 63% yield as a creamy white solid (0.359 g, 0.9759 mmol, mp 134-136° C.).

¹H-NMR (300 MHz, CDCl₃): δ 7.53 (d, 2H, J=9.0, H-2′), 7.23 (d, 1H, J=8.4, H-5), 7.12 (d, 2H, J=8.4, H-3′), 6.98 (s, 1H, H-4), 6.82 (d, 1H, J=2.1, H-8), 6.76 (d of d, 1H, J=8.4, 2.1, H-6), 5.95 (s, 1H, H-2), 4.00 (m, 1H, OCH₂CH₃), 3.78 (m, 1H, OCH₂CH₃) 2.32 (s, 3H, CH₃CO), 2.30 (s, 3H, CH₃CO), 1.25 (t, 3H, J=7.2, CH₂CH₃). ¹³C-NMR (75 MHz, CDCl₃): δ 169.5 (C═O), 169.3 (C═O), 151.4 (C7), 151.1 (C8a), 150.6 (C4), 134.6 (C^{a}), 129.7 (C^{a}), 128.0 (C5), 126.9 (C2′), 122.1 (C3′), 121.5 (C4), 119.6 (C4a), 115.4 (C6), 110.4 (C8), 97.2 (C2), 64.1 (CH₂CH₃), 21.5 (CH₃CO), 15.7 (CH₃CH₂).

C^{a}: C3 or C1′

MS (CI⁺): m/z 323 (isoflavylium, 100%). Microanalysis: Found: C=68.49%; H=5.53%; C₂₁H₂₀O₆ requires: C=68.40%; H=5.48%.

### Example 28

### 2-Ethoxy-4′,7-dihydroxy-isoflav-3-ene

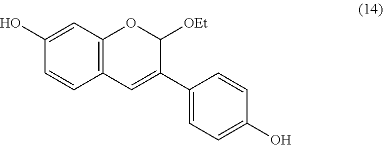

The diacetoxy compound of Example 27 (0.011 g, 0.03013 mmol) was stirred at rt in a 0.1M NaOH, 50% methanol/50% water solution (0.6 mL) and THF (4.5 mL) for 2 h. The solution was neutralised with 5M HCl and extracted with DCM (2×25 mL). The DCM layers were then combined, dried with MgSO₄ and concentrated, then applied to a silica plug. 15% Diethyl ether in DCM was passed through the plug to give the title product as a red glass (0.005 g, 0.016 mmol) in 53% yield.

¹H-NMR (300 MHz, d₄-methanol): δ 7.08 (d, 2H, J=8.4, H-2′), 7.00 (d, 1H, J=7.8, H-5), 6.81 (s, 1H, H-4), 6.48 (d, 2H, J=8.4, H-3′), 6.44 (d of d, 1H, J=7.8, 2.4, H-6), 6.35 (d, 1H, J=2.1, H-8), 6.13 (s, 1H, H-2), 4.80 (m, 1H, OCH₂CH₃), 3.63 (m, 1H, OCH₂CH₃). ¹³C-NMR (75 MHz, d₄-methanol): δ 158.7 (C8a), 156.9 (C7), 150.9 (C4′), 127.7 (C*), 126.8 (C*), 126.6 (C2′), 126.3 (C5), 119.5 (C4), 115.3 (C4a), 115.3 (C3′), 109.7 (C6), 103.6 (C8), 92.0 (C2), 67.8 (CH₂CH₃), 20.5 (CH₃CH₂).

C^{a}: C3 or C1′

MS (CI⁺): m/z 239.3 (isoflavylium, 100%). Microanalysis: Found: C=71.78%; H=4.21, N=5.25%; C₁₇H₁₆O₄ requires: C=71.82%; H=5.67%.

### Example 29

### 4′,7-Diacetoxy-2-(3-bromopropyloxy)-isoflav-3-ene

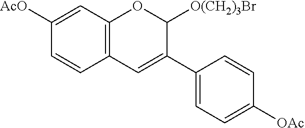

Freshly distilled anhydrous DCM (50 mL) was added to 4′,7-diacetoxy-isoflav-3-ene (0.434 g, 1.338 mmol), trityl hexafluororphosphate (0.9910 g, 2.570 mmol) and powdered 3 Å molecular sieves under argon. The murky brown solution was stirred at room temperature for 1 h. 3-Bromopropanol (1.2 mL) was then injected into the reaction mixture and the mixture stirred for 1.5 h. The solution was filtered. The filtrate was then concentrated by evaporation under reduced pressure and the residue applied to a silica column. A column was then run in 100% DCM. The title product was collected in 66% yield as a white solid (0.407 g, 0.8831 mmol, mp 123-125° C.).

¹H-NMR (300 MHz, CDCl₃): δ 7.54 (d, 2H, J=8.7, H-2′), 7.23 (d, 1H, J=8.4, H-5), 7.14 (d, 2H, J=8.7, H-3′), 7.00 (s, 1H, H-4), 6.85 (d, 1H, J=2.4, H-8), 6.78 (d of d, 1H, J=2.1, 8.1, H-6), 5.97 (s, 1H, H-2), 4.11 (m, 1H, OCH₂), 3.88 (m, 1H, OCH₂), 3.41 (m, 2H, BrCH₂), 2.31 (s, 3H, CH₃CO), 2.30 (s, 3H, CH₃CO), 2.11 (m, 2H, OCH₂CH₂CH₂Br). ¹³C-NMR (75 MHz, CDCl₃): δ 169.6 (C═O), 169.4 (C═O), 151.5 (C7), 151.0 (C8a), 150.7 (C4′), 134.3 (C^{a}), 129.5 (C^{a}), 128.1 (C5), 126.8 (CT), 122.2 (C3′), 121.4 (C4), 119.5 (C4a), 115.6 (C6), 110.4 (C8), 97.6 (C2), 65.6 (OCH₂), 32.7 (OCH₂CH₂), 30.8 (CH₂Br), 21.4 (COCH₃), 21.4 (COCH₃).

C^{a}: C3 or C1′

MS (CI⁺): m/z 463/461 (M+1, 13/17%), 462/460 (M⁺, 14/13%), 418/420 (M+1-COCH₃, 4/4%), 323 (M-OCH₂CH₂CH₂Br, 100%), 281 (M-OCH₂CH₂CH₂Br—COCH₃, 75%), 239 (M-OCH₂CH₂CH₂Br-2×COCH₃, 38%). HR (CI⁺) MS: m/z calcd for [M⁺] C₂₂H₂₁BrO₆: 461.0594, found: 461.0590.

### Example 30

### 2-(3-bromopropyloxy)-4′,7-dihydroxy-isoflav-3-ene

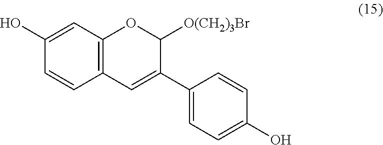

The diacetoxy 2-bromopropoxy compound from Example 29 (0.106 g, 0.231 mmol) was stirred at room temperature in a 0.1M NaOH, 50% methanol/50% water solution (1 mL) and THF (9 mL) for 20 h. An additional portion of aqueous 5M NaOH was added (0.2 mL) The solution was neutralised with 5M HCl_{aq} and extracted with DCM (2×50 mL). The DCM layers were then combined, dried with MgSO₄ and concentrated, then the residue applied to a silica plug. 15% Diethyl ether in DCM was passed through the plug to give the title product as a red glass (0.057 g, 0.1507 mmol) in 65% yield.

¹H-NMR (300 MHz, d₆-acetonitrile): δ 7.53 (d, 2H, J=8.7, H-2′), 7.23 (d, 1H, J=9.0, H-5), 7.08 (s, 1H, H-4), 6.96 (d, 2H, J=8.7, H-3′), 6.62 (m, 2H, H6, H-8), 6.11 (s, 1H, H-2), 4.16 (d of t, 1H, J=10.2, 5, OCH₂), 3.96 (d of d of d, 1H, J=12.3, 6.9, 5.7, OCH₂), 3.52 (t, 2H, J=6.6, OCH₂CH₂CH₂Br), 2.18 (t of d, 2H, J=6.5, 4.8, OCH₂CH₂CH₂Br). ¹³C-NMR (75 MHz, d₆-acetonitrile): δ 158.4 (C8a), 157.2 (C7), 151.7 (C4′), 129.0 (C3), 128.4 (C1′), 127.9 (C5), 127.1 (CT), 119.5 (C4), 116.0 (C3′), 115.3 (C4a), 109.8 (C6), 103.8 (C8), 97.8 (C2), 65.7 (OCH₂), 33.1 (CH₂CH₂Br), 31.2 (CH₂Br). (CI⁺) MS: m/z 379.3/377.1 (M+1, 9/10%), 239 (M-O(CH₂)₃Br, 100%). HR(CI⁺) MS: m/z calcd for [M⁺] C₁₈H₁₇BrO₄: 377.0383, found: 377.0387.

### Example 31

### 4′,7-Diacetoxy-2-hydroxy-isoflav-3-ene

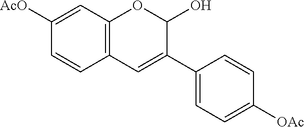

Anhydrous DCM is added to 4′,7-diacetoxy-isoflav-3-ene, trityl hexafluorophosphate and powdered 3 Å molecular sieves. The brown-yellow solution is stirred at room temperature for 30 min. Wet tetrahydrofuran is then added to the reaction mixture, stirred and the resultant mixture is filtered. The filtrate is dried (MgSO₄), dissolved into DCM and subjected to column chromatography to afford the title compound.

### Example 32

### 2,4′,7-Trihydroxy-isoflav-3-ene

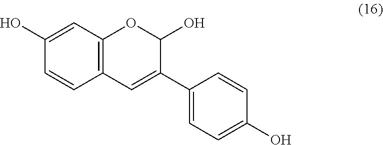

The diacetoxy compound of Example 31 is deprotected according to the general method of Example 18 to afford the title compound.

Alternatively, the title compound can be prepared as follows.

To a stirred solution of phenoxodiol (250 mg, 1.04 mmol) in TFA (5 mL) was added thallium(III) trifluoroacetate (TTFA) (600 mg, 1.10 mmol). The mixture was stirred further for 15 min, poured into water (120 mL) and extracted with ethyl acetate (50 mL×1, 25 mL×2). The combined organic extract was washed with saturated sodium bicarbonate solution (50 mL×2), dried over anhydrous sodium sulfate and concentrated under vacuum. The crude product was adsorbed on silica gel. Chromatography (SiO₂, 40% ethyl acetate/hexanes) gave trihydroxyisoflav-3-ene as a pink solid (130 mg, 48%). M.p.>325° C.; UV (MeOH): λₘₐₓ 211 (ε 22853 cm⁻¹M⁻¹), 236 (ε 11623 cm⁻¹M⁻¹), 323 (ε 26597 cm⁻¹M⁻¹) nm; IR (KBr): νₘₐₓ 3228 (br), 3228 (br), 1814, 1623, 1610, 1589, 1518, 1508, 1286, 1257, 1129, 983, 963 cm⁻¹; ¹H NMR (300 MHz, acetone-d₆): δ 5.91 (d, J=7.1 Hz, 1H, 2OH), 6.21 (d, J=7.1 Hz, 1H, H2), 6.45 (d, J=2.6 Hz, 1H, H8), 6.48 (dd, J=2.6, 8.3 Hz, 1H, H6), 6.85 (d, J=8.7 Hz, 2H, H3′, H5′), 6.88 (s, 1H, H4′), 7.08 (d, J=8.3 Hz, 1H, H5), 7.50 (d, J=8.7 Hz, 2H, H2′, H6′), 8.37 (s, 1H, 4′ OH), 8.42 (s, 1H, 7 OH); ¹³C NMR (75.6 MHz, acetone-d₆): δ 91.6 (C2), 103.3 (C8), 108.7 (C6), 114.4 (C4a), 115.3 (C3′, C5′), 118.0 (C4), 126.5 (CT, C6′), 127.6 (C5), 128.7 and 129.0 (C3 and C1′), 151.6 (C8a), 156.9 (C4′), 158.2 (C7); HRMS (ESI) m/z Calcd for C₁₅H₁₂O₄Na (M+Na)⁺279.0628. Found 279.0630.

### Example 33

### 4′,7-Diacetoxy-2-(N-benzyl)methyl-isoflav-3-ene

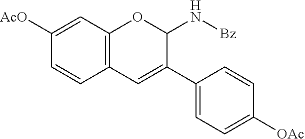

4′,7-diacetoxy-isoflav-3-ene (260 mg, 0.80 mmol) and tritylium hexafluorophosphate (380 mg, 0.98 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Benzylamine (0.1 ml, 0.92 mmol) was added to the stirring suspension under an atmosphere of nitrogen. The reaction mixture was stirred at room temperature for 17 hours, after which the volume was concentrated in vacuo to give the title compound as a clear yellow solid (yield 190 mg, 55%).

¹H NMR (400 MHz, CDCl₃) δ 7.58 (2H, d, J=8.7 Hz, H2′,6′), 7.36-7.17 (5H, m, benzyl Ar), 7.14 (1H, d, J=8.4 Hz, H5), 7.08 (2H, d, J=8.7 Hz, H3′, 5′), 6.88 (1H, br s, H4), 6.79 (1H, d, J=2.2 Hz, H8), 6.72 (1H, dd, J=2.2 Hz, 8.4 Hz, H6), 5.69 (1H, br s, H2), 4.07 (2H, dd, J=13.5 Hz, 24.2 Hz, benzyl CH₂), 2.32 (3H, s, acetate CH₃), 2.30 (3H, s, acetate CH₃).

### Example 34

### 4′,7-Dihydroxy-2-(N-benzyl)methyl-isoflav-3-ene

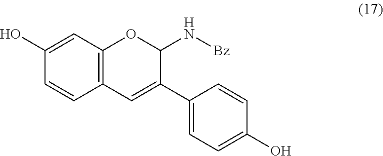

The diacetoxy compound of Example 33 is deprotected according to the general method of Example 18 to afford the title compound.

### Example 35

### 4′,7-Diacetoxy-2-ethylthio-isoflav-3-ene

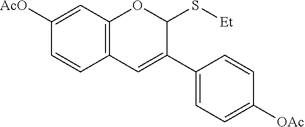

4′,7-diacetoxy-isoflav-3-ene (260 mg, 0.80 mmol) and tritylium hexafluorophosphate (400 mg, 1.03 mmol) were dissolved in dry dichloromethane (100 ml). The reaction mixture was stirred at room temperature, under nitrogen, for one hour. The yellow solid that precipitated during this time was isolated via vacuum filtration and resuspended in dry dichloromethane (100 ml). Ethanethiol (0.1 ml, 1.35 mmol) was added to the stirring suspension under an atmosphere of nitrogen. The reaction mixture was stirred at room temperature for 17 hours, after which the volume was concentrated in vacuo to give the title compound as a red/orange solid (yield 160 mg, 52%).

¹H NMR (400 MHz, CDCl₃) δ 7.61 (2H, d, J=8.8 Hz, H2′, 6′), 7.16 (1H, d, J=8.4 Hz, H5), 7.13 (2H, d, J=8.8 Hz, H3′, 5′), 6.86 (1H, br s, H4), 6.68 (1H, dd, J=2.2 Hz, 8.4 Hz, H6), 6.75 (1H, d, J=2.2 Hz), 6.46 (1H, br s, H2), 2.85-2.65 (2H, m, thioethyl CH₂), 2.32 (3H, s, acetate CH₃), 2.30 (3H, s, acetate CH₃), 1.35 (3H, t, J=7.3 Hz, thioethyl CH₃).

### Example 36

### 2-Ethylthio-4′,7-dihydroxy-isoflav-3-ene

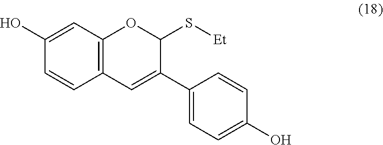

The diacetoxy compound of Example 35 is deprotected according to the general method of Example 18 to afford the title compound.

### Example 37

### 4′,7-Diacetoxy-2-methylthio-isoflav-3-ene

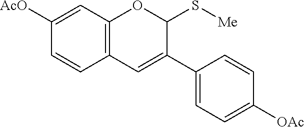

Freshly distilled anhydrous DCM is added to 4′,7-diacetoxy-isoflav-3-ene, trityl hexafluororphosphate and powdered 3 Å molecular sieves under argon. The murky brown solution is stirred at room temperature for 1 h. Methylthiol is then added to the reaction mixture and the mixture stirred for 1.5 h. The solution is filtered, concentrated by evaporation under reduced pressure and purified by column chromatography to afford the title compound.

### Example 38

### 4′,7-Dihydroxy-2-methylthio-isoflav-3-ene

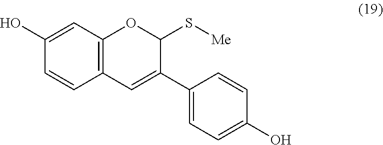

The diacetoxy compound of Example 37 is deprotected according to the general method of Example 18 to afford the title compound.

### Example 39

### 4′,7-Diacetoxy-2-(triazo-3-yl)-isoflav-3-ene

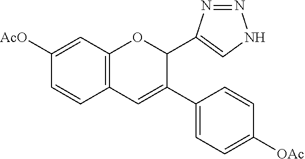

The ethynyl diacetate compound of Example 21 is subjected to azide 1,3-cycloaddition to the acetylene unit to prepare the title compound.

### Example 40

### 4′,7-Dihydroxy-2-(triazo-3-yl)-isoflav-3-ene

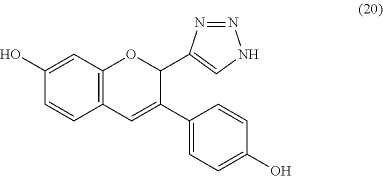

The diacetoxy compound of Example 39 is deprotected according to the general method of Example 18 to afford the title compound.

### Example 41

### 4′,7-Diacetoxy-2-(pyridin-3-yl)-isoflav-3-ene

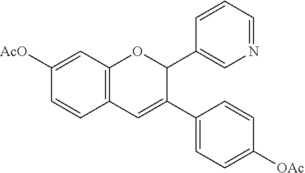

Freshly distilled anhydrous DCM is added to 4′,7-diacetoxy-isoflav-3-ene, trityl hexafluorophosphate and powdered 3 Å molecular sieves under argon. The murky brown solution is stirred at room temperature for 1 h. 3-Trimethylsilylpyridine is then added to the reaction mixture and the mixture stirred for 1.5 h. The solution is filtered, concentrated by evaporation under reduced pressure and purified by column chromatography to afford the title compound.

### Example 42

### 4′,7-Dihydroxy-2-(pyridin-3-yl)-isoflav-3-ene

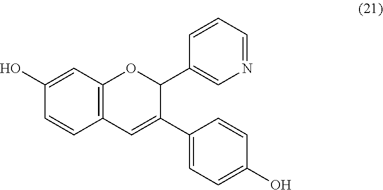

The diacetoxy compound of Example 41 is deprotected according to the general method of Example 18 to afford the title compound.

### Example 43

### 4′,7-Dihydroxy-2-(pyridazin-4-yl)-isoflav-3-ene

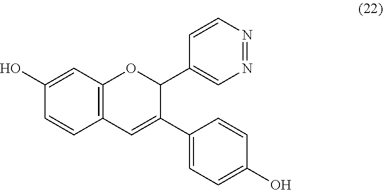

The title compound is prepared according to the general method of Example 17 by reacting the isoflavylium salt of 4′,7-diacetoxy-isoflav-3-ene with 4-trimethylsilylpyridazine and subsequent deprotection according to the general method of Example 18.

### Example 44

### 4′,7-Dihydroxy-2-(pyrimidin-5-yl)-isoflav-3-ene

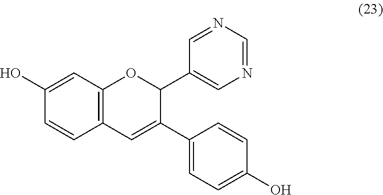

The title compound is prepared according to the general method of Example 17 by reacting the isoflavylium salt of 4′,7-diacetoxy-isoflav-3-ene with 5-trimethylsilylpyrimidine and subsequent deprotection according to the general method of Example 18.

### Example 45

### 4′,7-Dihydroxy-2-(pyrazin-2-yl)-isoflav-3-ene

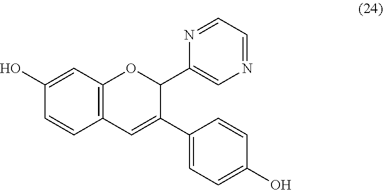

The title compound is prepared according to the general method of Example 17 by reacting the isoflavylium salt of 4′,7-diacetoxy-isoflav-3-ene with 2-trimethylsilylpyrazine and subsequent deprotection according to the general method of Example 18.

### Example 46

### 4′,7-Dihydroxy-2-(pyridin-2-yl)-isoflav-3-ene

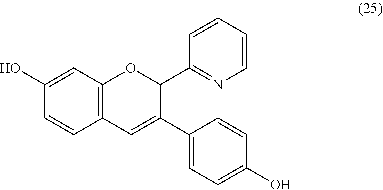

The title compound is prepared according to the general method of Example 17 by reacting the isoflavylium salt of 4′,7-diacetoxy-isoflav-3-ene with 2-trimethylsilylpyridine and subsequent deprotection according to the general method of Example 18.

### Example 47

### 4′,7-Dihydroxy-2-(pyridin-4-yl)-isoflav-3-ene

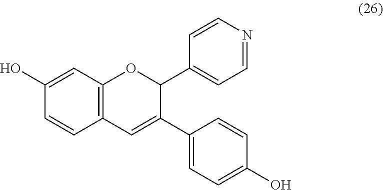

The title compound is prepared according to the general method of Example 17 by reacting the isoflavylium salt of 4′,7-diacetoxy-isoflav-3-ene with 4-trimethylsilylpyridine and subsequent deprotection according to the general method of Example 18.

### Example 48

### 4′,7-Dihydroxy-2-(pyrrol-2-yl)-isoflav-3-ene

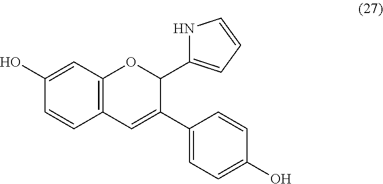

The title compound is prepared according to the general method of Example 17 by reacting the isoflavylium salt of 4′,7-diacetoxy-isoflav-3-ene with 2-trimethylsilylpyrrole and subsequent deprotection according to the general method of Example 18.

### Example 49

### 4′,7-Dihydroxy-2-(imidazol-4-yl)-isoflav-3-ene

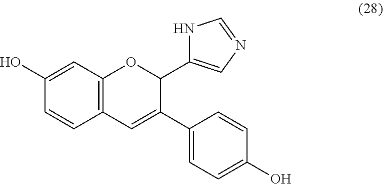

The title compound is prepared according to the general method of Example 17 by reacting the isoflavylium salt of 4′,7-diacetoxy-isoflav-3-ene with 4-trimethylsilylimidazole and subsequent deprotection according to the general method of Example 18.

### Example 50

### 4′,7-Dihydroxy-2-(1,2,4-triazol-3-yl)-isoflav-3-ene

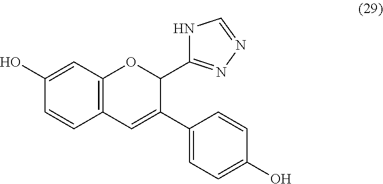

The title compound is prepared according to the general method of Example 17 by reacting the isoflavylium salt of 4′,7-diacetoxy-isoflav-3-ene with 3-trimethylsilyl-1,2,4-triazole and subsequent deprotection according to the general method of Example 18.

### Example 51

### 4′,7-Dihydroxy-2-(tetrazol-4-yl)-isoflav-3-ene

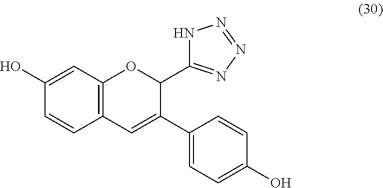

The title compound is prepared according to the general method of Example 17 by reacting the isoflavylium salt of 4′,7-diacetoxy-isoflav-3-ene with 5-trimethylsilyltetrazole and subsequent deprotection according to the general method of Example 18.

### Example 52

### 4′,7-Dihydroxy-2-(1,2,4-triazin-6-yl)-isoflav-3-ene

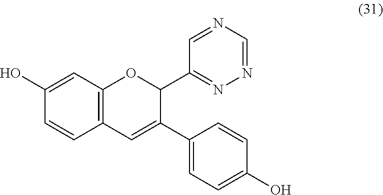

The title compound is prepared according to the general method of Example 17 by reacting the isoflavylium salt of 4′,7-diacetoxy-isoflav-3-ene with 6-trimethylsilyl-1,2,4-triazine and subsequent deprotection according to the general method of Example 18.

### Example 53

### 4′,7-Dihydroxy-2-(1,2,3,4-tetrazin-4-yl)-isoflav-3-ene

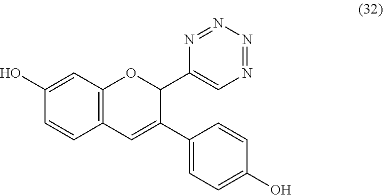

The title compound is prepared according to the general method of Example 17 by reacting the isoflavylium salt of 4′,7-diacetoxy-isoflav-3-ene with 5-trimethylsilyl-1,2,3,4-tetrazine and subsequent deprotection according to the general method of Example 18.

### Anhydride Cyclisations

2-Substituted isoflavonoid compounds of the subject invention are also available by anhydride cyclisation with 1,2-diphenyl-ethanones.

### Example 54

### 3-(4-Hydroxy-phenyl)-2-pyridin-4-yl-2H-chromen-7-ol (26)

### Example 54(a)

### 7-Hydroxy-3-(4-hydroxy-phenyl)-2-pyridin-4-yl-chromen-4-one

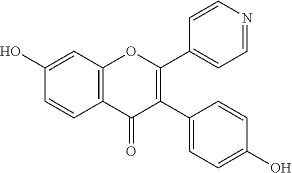

1-(2,4-Dihydroxy-phenyl)-2-(4-hydroxy-phenyl)-ethanone (1.06 g, 4.35 mmol) and isonicotinic anhydride (3.27 g, 14.1 mmol) were dissolved in triethylamine (10 ml). The solution was heated to reflux for 22 hours. Once the reaction mixture had cooled, it was poured into water (60 ml), acidified (to pH 5) with 2M HCl and stirred at room temperature for two hours. A yellow-brown precipitate was collected by vacuum filtration and refluxed in methanol (5 ml) with sodium hydroxide solution (2M, 5 ml) for 45 minutes.

The mixture was allowed to cool, then poured into water (50 ml), neutralised with 2M HCl, and stirred at room temperature overnight. Vacuum filtration afforded the title compound as a yellow solid (Yield: 832 mg, 58%).

¹H NMR (400 MHz in DMSO) δ 8.53 (2H, d, J=6.1 Hz, H-3″,5″), 7.90 (1H, d, J=8.8 Hz, H-5), 7.31 (2H, d, J=6.1 Hz, H-2″,6″), 6.93 (2H, d, J=8.6 Hz, H-2′,6′), 6.89 (1H, dd, J=1.9, 8.7 Hz, H-6), 6.84 (1H, d, J=2.1 Hz), 6.65 (2H, d, J=8.6 Hz, H-3′,5′).

### Example 54(b)

### Acetic acid 3-(4-acetoxy-phenyl)-4-oxo-2-pyridin-4-yl-4H-chromen-7-yl ester

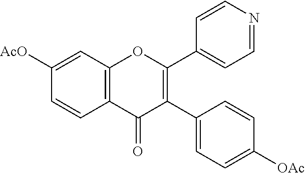

7-Hydroxy-3-(4-hydroxy-phenyl)-2-pyridin-4-yl-chromen-4-one (723 mg, 2.18 mmol) and potassium carbonate (664 mg, 4.80 mmol) were refluxed in acetone (15 ml) for one hour. Once cooled, the reaction mixture was poured into water (30 ml) and neutralised with 2M HCl. Vacuum filtration afforded the title compound as a pale yellow solid in quantitative yield.

¹H NMR (400 MHz in DMSO) δ 8.59 (2H, d, J=6.1 Hz, H-3″,5″), 8.16 (1H, d, J=8.7 Hz, H-5), 7.62 (1H, d, J=2.1 Hz, H-8), 7.36 (2H, d, J=6.2 Hz, H-2″,6″), 7.35 (1H, dd, J=1.9, 8.3 Hz, H-6), 7.23 (2H, d, J=8.7 Hz, H-2′,6′), 7.09 (2H, d, J=8.7 Hz, H-3′,5′), 2.34 (3H, s, acetate CH₃), 2.25 (3H, s, acetate CH₃).

### Example 54(c)

### Acetic acid 3-(4-acetoxy-phenyl)-4-hydroxy-2-pyridin-4-yl-chroman-7-yl ester

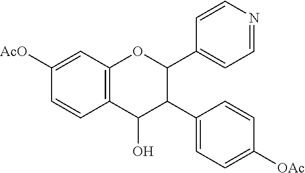

Acetic acid 3-(4-acetoxy-phenyl)-4-oxo-2-pyridin-4-yl-4H-chromen-7-yl ester (611 mg, 1.47 mmol) and platinum(IV) oxide hydrate (1.77 g) were suspended in ethyl acetate (20 ml). The reaction mixture was stirred under hydrogen (1 bar) for 8 hours. The catalyst was removed via vacuum filtration. The solvent was evaporated in vacuo to give the title compound as a yellow solid (Yield: 267 mg, 43%).

¹H NMR (400 MHz, CDCl₃) δ 8.46 (2H, d, J=6.0 Hz, H-3″,5″), 7.60 (1H, d, J=8.4 Hz, H-5), 7.11 (2H, d, J=5.6 Hz, H-2″,6″), 6.92 (2H, d, J=8.8 Hz, H-2′,6′), 6.85 (2H, d, J=8.9 Hz, H-3′,5′), 6.83 (1H, dd, J=2.2, 8.5 Hz, H-6), 6.79 (1H, d, J=2.3 Hz, H-8), 5.56 (1H, br d, J=2.4 Hz, H-2), 5.45 (1H, br d, J=7.1 Hz, H-4), 3.67 (1H, dd, J=2.5, 7.0 Hz, H-3), 2.32 (3H, s, acetate CH₃), 2.23 (3H, s, acetate CH₃).

### Example 54(d)

### Acetic acid 3-(4-acetoxy-phenyl)-2-pyridin-4-yl-2H-chromen-7-yl ester

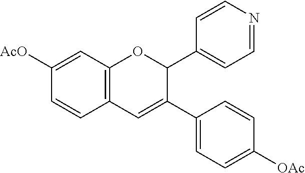

A suspension of acetic acid 3-(4-acetoxy-phenyl)-4-hydroxy-2-pyridin-4-yl-chroman-7-yl ester (101 mg, 0.24 mmol) and phosphorous pentoxide (880 mg, 6.2 mmol) in dichloromethane (5 ml) was stirred at room temperature for 16 hours, in a flask fitted with a drying tube. The dichloromethane was decanted and the residue was dissolved in methanol (20 ml) and poured into water (100 ml), with stirring. Saturated NaHCO₃ solution (10 ml) was added prior to extraction with ethyl acetate (3×20 ml). The organic layer were washed with brine (30 ml) and dried over MgSO₄. The solvent was evaporated in vacuo to give the title compound as a yellow solid (Yield: 81 mg, 84%).

¹H NMR (400 MHz in DMSO) δ 8.51 (2H, d, J=6.1 Hz, H-3″,5″), 7.65 (2H, d, J=8.8 Hz, H-2′,6′), 7.41 (1H, br s, H-4), 7.34 (2H, d, J=6.2 Hz, H-2″,6″), 7.30 (1H, d, J=8.3 Hz, H-5), 7.16 (2H, d, J=8.8 Hz, H-3′,5′), 6.71 (1H, dd, J=2.2, 8.1 Hz, H-6), 6.67 (1H, d, J=2.3 Hz, H-8), 6.66 (1H, br s, H-2), 2.26 (3H, s, acetate CH₃), 2.21 (3H, s, acetate CH₃).

### Example 54(e)

### 3-(4-Hydroxy-phenyl)-2-pyridin-4-yl-2H-chromen-7-ol

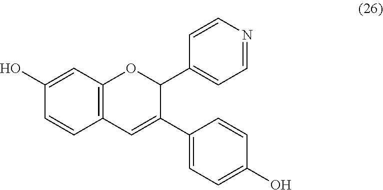

To a solution of acetic acid 3-(4-acetoxy-phenyl)-2-pyridin-4-yl-2H-chromen-7-yl ester (81 mg, 0.20 mmol) in methanol (3 ml), 1M potassium hydroxide solution (0.2 ml) was added. The mixture was stirred for 15 minutes at room temperature before it was neutralised with 1M acetic acid. Water (10 ml) was added and the resulting mixture was stirred at room temperature for 2 hours. Vacuum filtration afforded the title compound as a pale orange powder (23 mg, 36%).

¹H NMR (400 MHz in DMSO) δ 9.61 (1H, br s, OH), 9.52 (1H, br s, OH), 8.47 (2H, d, J=6.1 Hz, H-3″,5″), 7.38 (2H, d, J=8.9 Hz, H-2′,6′), 7.26 (2H, d, J=6.1 Hz, H-2″,6″), 7.07 (1H, br s, H-4), 7.01 (1H, d, J=8.3 Hz, H-5), 6.74 (2H, d, J=8.8 Hz, H-3′,5′), 6.42 (1H, br s, H-2), 6.32 (1H, dd, J=2.3, 8.2 Hz, H-6), 6.22 (1H, d, J=2.3 Hz, H-8).

### Example 55

### 3-(4-Hydroxy-phenyl)-2-propyl-2H-chromen-7-ol (33)

### Example 55(a)

### 7-Hydroxy-3-(4-hydroxy-phenyl)-2-propyl-chromen-4-one

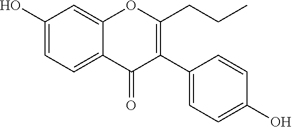

1-(2,4-Dihydroxy-phenyl)-2-(4-hydroxy-phenyl)-ethanone (1.04 g, 4.27 mmol) and butyric anhydride (2.2 ml, 13.4 mmol) were dissolved in triethylamine (10 ml). The solution was heated to reflux for 22 hours. Once the reaction mixture had cooled, it was poured into water (60 ml), acidified (to pH 5) with 2M HCl and stirred at room temperature for two hours. A brown solid was collected by vacuum filtration and refluxed in methanol (5 ml) with sodium hydroxide solution (2M, 5 ml) for 45 minutes. The mixture was allowed to cool, then poured into water (50 ml), neutralised with 2M HCl, and stirred at room temperature overnight. Vacuum filtration afforded the title compound as a pale orange solid in quantitative yield.

¹H NMR (400 MHz in DMSO) δ 7.81 (1H, d, J=8.7 Hz, H-5), 6.95 (2H, d, J=8.6 Hz, H-2′,6′), 6.84 (1H, dd, J=2.2, 8.7 Hz, H-6), 6.79 (1H, d, J=2.2 Hz, H-8), 6.75 (2H, d, J=8.6 Hz, H-3′,5′), 2.41 (2H, br t, J=7.5 Hz, CH₂CH₂CH₃), 1.56 (2H, sextet, J=7.5 Hz, CH₂CH₂CH₃), 0.77 (3H, t, J=7.4 Hz, CH₂CH₂CH₃).

### Example 55(b)

### Acetic acid 3-(4-acetoxy-phenyl)-4-oxo-2-propyl-4H-chromen-7-yl ester

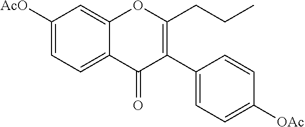

7-Hydroxy-3-(4-hydroxy-phenyl)-2-propyl-chromen-4-one (1.24 g, 4.18 mmol) and potassium carbonate (1.74 g mg, 12.6 mmol) were refluxed in acetone (15 ml) for one hour. Once cooled, the reaction mixture was poured into water (30 ml) and neutralised with 2M HCl. Vacuum filtration afforded the title compound as a beige solid (Yield: 976 mg, 61%).

¹H NMR (400 MHz in DMSO) δ 8.08 (1H, d, J=8.7 Hz, H-5), 7.53 (1H, d, J=2.1 Hz, H-8), 7.29 (2H, d, J=8.6 Hz, H-2′,6′), 7.27 (1H, dd, J=2.3, 8.5 Hz, H-6), 7.20 (2H, d, J=8.6 Hz, H-3′,5′), 2.52 (2H, br tr, J=7.5 Hz, CH₂CH₂CH₃), 2.33 (3H, s, acetate CH₃), 2.29 (3H, s, acetate CH₃), 1.66 (2H, sextet, J=7.3 Hz, CH₂CH₂CH₃), 0.83 (3H, t, J=7.4 Hz, CH₂CH₂CH₃).

### Example 55(c)

### Acetic acid 3-(4-acetoxy-phenyl)-4-hydroxy-2-propyl-chroman-7-yl ester

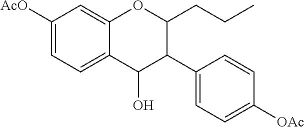

Acetic acid 3-(4-acetoxy-phenyl)-4-oxo-2-propyl-4H-chromen-7-yl ester (517 mg, 1.36 mmol) and 5% palladium on carbon paste (2.76 g) were suspended in ethyl acetate (10 ml). The reaction mixture was stirred under hydrogen (1 bar) for one week. The catalyst was removed via vacuum filtration through a plug of Celite. The solvent was evaporated in vacuo to give the title compound (a mixture of cis and trans isomers around the C-3-C-4 bond) as an off-white solid (Yield: 336 mg, 64%).

¹H NMR (400 MHz, CDCl₃) δ 7.51 (1H, d, J=8.5 Hz, trans H-5), 7.49 (1H, d, J=8.4 Hz, cis H-5), 7.22 (2H, d, J=8.6 Hz, trans H-2′,6′), 7.14 (2H, d, J=8.7 Hz, cis H-2′,6′), 7.01 (2H, d, J=8.7 Hz, cis H-3′,5′), 6.99 (2H, d, J=8.7 Hz, trans H-3′,5′), 6.71 (1H, dd, J=2.3, 8.4 Hz, trans H-6), 6.69 (1H, dd, J=2.3, 8.4 Hz, cis H-6), 6.64 (1H, d, J=2.3 Hz, trans H-8), 6.63 (1H, d, J=2.3 Hz, cis H-8), 5.18 (1H, br d, J=6.9 Hz, cis H-4), 4.94 (1H, d, J=10.0 Hz, trans H-4), 4.43 (1H, dd, J=2.2, 4.9, 8.1 Hz, cis H-2), 4.26 (1H, ddd, J=2.5, 6.4, 8.1 Hz, trans H-2), 3.36 (1H, dd, J=2.3, 7.1 Hz, cis H-3), 2.85 (1H, dd, J=10.3, 10.3 Hz, trans H-3), 2.32 (3H, s, trans acetate CH₃), 2.30 (3H, s, cis acetate CH₃), 2.29 (3H, s, trans acetate CH₃), 2.27 (3H, s, cis acetate CH₃), 1.62-1.30 (8H, m, cis and trans CH₂CH₂CH₃), 0.92 (3H, t, cis CH₂CH₂CH₃), 0.89 (3H, t, trans CH₂CH₂CH₃).

### Example 55(d)

### Acetic acid 3-(4-acetoxy-phenyl)-2-propyl-2H-chromen-7-yl ester

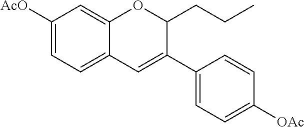

Acetic acid 3-(4-acetoxy-phenyl)-4-hydroxy-2-propyl-chroman-7-yl ester (305 mg, 0.79 mmol) and 85% phosphoric acid (0.75 ml) were refluxed in toluene (7.5 ml) for 19 hours. The reaction mixture was allowed to cool, neutralised with saturated sodium hydrogen carbonate solution and extracted with ethyl acetate (3×20 ml). Semi-preparative HPLC gave the title compound as a brown solid (Yield: 48 mg, 16%).

¹H NMR (400 MHz, CDCl₃) δ 7.46 (2H, d, J=8.8 Hz, H-2′,6′), 7.11 (2H, d, J=8.8 Hz, H-3′,5′), 7.06 (1H, d, J=8.0 Hz, H-5), 6.68 (1H, br s, H-4), 6.65 (1H, dd, J=2.3, 8.0 Hz, H-6), 6.63 (1H, d, J=2.2 Hz, H-8), 5.29 (1H, dd, J=2.5, 9.9 Hz, H-2), 2.32 (3H, s, acetate CH₃), 2.29 (3H, s, acetate CH₃), 1.90-1.79 (1H, m, CH^{a}CH₂CH₃), 1.50-1.38 (3H, m, CH^{b}CH₂CH₃), 0.89 (3H, t, J=7.2 Hz, CH₂CH₂CH₃).

### Example 55(e)

### 3-(4-Hydroxy-phenyl)-2-propyl-2H-chromen-7-ol (33)

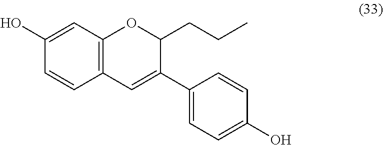

To a solution of acetic acid 3-(4-acetoxy-phenyl)-2-propyl-2H-chromen-7-yl ester (48 mg, 0.13 mmol) in methanol (5 ml), 1M potassium hydroxide solution (0.5 ml) was added. The mixture was stirred for 15 minutes at room temperature before it was neutralised with 1M acetic acid. Water (20 ml) was added and the resulting mixture was extracted with ethyl acetate (3×5 ml). Solvent was evaporated in vacuo to give the title compound as a brown solid in quantitative yield.

¹H NMR (400 MHz in d₆-DMSO) δ 9.56 (1H, br s, OH), 9.52 (1H, br s, OH), 7.36 (2H, d, J=8.8 Hz, H-2′,6′), 6.93 (1H, d, J=8.2 Hz, H-5), 6.77 (1H, d J=8.8 Hz, H-3′,5′), 6.70 (1H, br s, H-4), 6.31 (1H, dd, J=2.3, 8.1 Hz, H-6), 6.24 (1H, d, J=2.2 Hz, H-8), 5.26 (1H, dd, J=2.9, 9.8 Hz, H-2), 1.51-1.27 (4H, m, CH₂CH₂CH₃), 0.85 (3H, t, J=7.3 Hz, CH₂CH₂CH₃).

### Example 56

### 3-(4-Hydroxy-phenyl)-2-isopropyl-2H-chromen-7-ol (34)

### Example 56(a)

### 7-Hydroxy-3-(4-hydroxy-phenyl)-2-isopropyl-chromen-4-one

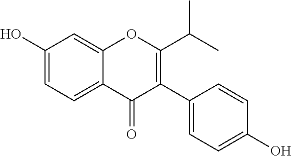

1-(2,4-Dihydroxy-phenyl)-2-(4-hydroxy-phenyl)-ethanone (0.98 g, 4.02 mmol) and isobutyric anhydride (2.2 ml, 13.3 mmol) were dissolved in triethylamine (10 ml). The solution was heated to reflux for 22 hours. Once the reaction mixture had cooled, it was poured into water (60 ml), acidified (to pH 5) with 2M HCl and stirred at room temperature for two hours. A brown solid was collected by vacuum filtration and refluxed in methanol (5 ml) with sodium hydroxide solution (2M, 5 ml) for 45 minutes. The mixture was allowed to cool, then poured into water (50 ml), neutralised with 2M HCl, and stirred at room temperature overnight. Vacuum filtration afforded the title compound as a beige solid (Yield: 855 mg, 72%).

¹H NMR (400 MHz in DMSO) δ 7.84 (1H, d, J=8.7 Hz, H-5), 6.99 (2H, d, J=8.5 Hz, H-2′,6′), 6.88 (1H, dd, J=2.2, 8.7 Hz, H-6), 6.85 (1H, d, J=2.2 Hz, H-8), 6.80 (2H, d, J=8.5 Hz, H-3′,5′), 2.84 (1H, septet, J=6.8 Hz, CH(CH₃)₂), 1.17 (6H, d, J=6.9 Hz, CH(CH₃)₂).

### Example 56(b)

### Acetic acid 3-(4-acetoxy-phenyl)-2-isopropyl-4-oxo-4H-chromen-7-yl ester

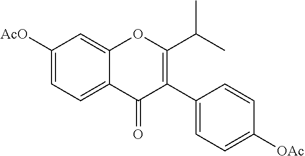

7-Hydroxy-3-(4-hydroxy-phenyl)-2-isopropyl-chromen-4-one (779 mg, 2.63 mmol) and potassium carbonate (801 mg, 5.80 mmol) were refluxed in acetone (15 ml) for one hour. Once cooled, the reaction mixture was poured into water (30 ml) and neutralised with 2M HCl. Vacuum filtration afforded the title compound as an off-white solid (Yield: 775 mg, 77%).

¹H NMR (400 MHz in DMSO) δ 8.07 (1H, d, J=8.6 Hz, H-5), 7.56 (1H, d, J=2.1 Hz, H-8), 7.29 (2H, d, J=8.7 Hz, H-2′,6′), 7.27 (1H, dd, J=2.1, 8.6 Hz, H-6), 7.20 (2H, d, J=8.7 Hz, H-3′,5′), 2.82 (1H, septet, J=6.9 Hz, CH(CH₃)₂), 2.33 (3H, s, acetate CH₃), 2.29 (3H, s, acetate CH₃), 1.22 (6H, d, J=6.8 Hz, CH(CH₃)₂).

### Example 56(c)

### Acetic acid 3-(4-acetoxy-phenyl)-4-hydroxy-2-isopropyl-chroman-7-yl ester

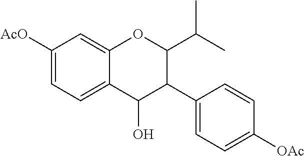

Acetic acid 3-(4-acetoxy-phenyl)-2-isopropyl-4-oxo-4H-chromen-7-yl ester (497 mg, 1.31 mmol) and 5% palladium on carbon paste (2.44 g) were suspended in ethyl acetate (10 ml). The reaction mixture was stirred under hydrogen (1 bar) for one week. The catalyst was removed via vacuum filtration through a plug of Celite. The solvent was evaporated in vacuo to give the title compound (a mixture of cis and trans isomers around the C-3-C-4 bond) as an off-white solid (Yield: 381 mg, 63%).

¹H NMR (400 MHz, CDCl₃) δ 7.50 (1H, d J=8.2 Hz, trans H-5), 7.46 (1H, d J=8.2 Hz, cis H-5), 7.26 (2H, d, J=8.7 Hz, trans H-2′,6′), 7.17 (2H, d, J=8.7 Hz, cis H-2′,6′), 6.99 (2H, d, J=8.8 Hz, cis H-3′,5′), 6.97 (2H, d, J=8.9 Hz, trans H-3′,5′), 6.69 (1H, dd, J=2.3, 8.1 Hz, cis H-6), 6.68 (1H, dd, J=2.3, 8.2 Hz, trans H-6), 6.67 (1H, d, J=2.1 Hz, cis H-8), 6.65 (1H, d, J=2.2 Hz, trans H-8), 5.13 (1H, br d, J=7.0 Hz, cis H-4), 4.93 (1H, br d, J=10.0 Hz, trans H-4), 4.18 (1H, dd, J=2.0, 11.0 Hz, cis H-2), 3.96 (1H, dd, J=2.1, 10.1 Hz, trans H-2), 3.54 (1H, dd, J=2.4, 6.8 Hz, cis H-3), 2.95 (1H, dd, J=10.4, 10.4 Hz, trans H-3), 2.32 (3H, s, trans acetate CH₃), 2.30 (3H, s, cis acetate CH₃), 2.29 (3H, s, trans acetate CH₃), 2.26 (3H, s, cis acetate CH₃), 1.69 (1H, d septet, J=7.4, 12.3 Hz, trans CH(CH₃)₂), 1.58 (1H, d septet, J=2.6, 7.0 Hz, cis CH(CH₃)₂), 1.07 (6H, d, J=6.8 Hz, cis CH(CH₃)₂), 1.03 (6H, d, J=7.0 Hz, trans CH(CH₃)₂).

### Example 56(d)

### Acetic acid 3-(4-acetoxy-phenyl)-2-isopropyl-2H-chromen-7-yl ester

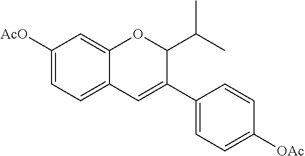

Acetic acid 3-(4-acetoxy-phenyl)-4-hydroxy-2-isopropyl-chroman-7-yl ester (300 mg, 0.78 mmol) and 85% phosphoric acid (0.75 ml) were refluxed in toluene (7.5 ml) for 19 hours. The reaction mixture was allowed to cool, neutralised with saturated sodium hydrogen carbonate solution and extracted with ethyl acetate (3×20 ml). Semi-preparative HPLC gave the title compound as a brown solid (Yield: 49 mg, 17%).

¹H NMR (400 MHz, CDCl₃) δ 7.45 (2H, d, J=8.8 Hz, H-2′,6′), 7.10 (2H, d, J=8.8 Hz, H-3′,5′), 7.08 (1H, d, J=8.0 Hz, H-5), 6.67 (1H, dd, J=2.3, 8.0 Hz, H-6), 6.62 (1H, br s, H-4), 6.59 (1H, d, J=2.1 Hz, H-8), 5.15 (1H, d, J=5.6 Hz, H-2), 2.32 (3H, s, acetate CH₃), 2.29 (3H, s, acetate CH₃), 1.95 (1H, d septet, J=5.6, 7.1 Hz, CH(CH₃)₂), 0.88 (3H, d, J=6.8 Hz, CH(CH₃)₂), 0.85 (3H, d, J=6.9 Hz, CH(CH₃)₂).

### Example 56(e)

### 3-(4-Hydroxy-phenyl)-2-isopropyl-2H-chromen-7-ol (34)

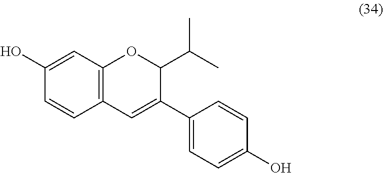

To a solution of acetic acid 3-(4-acetoxy-phenyl)-2-isopropyl-2H-chromen-7-yl ester (0.49 mg, 0.13 mmol) in methanol (5 ml), 1M potassium hydroxide solution (0.5 ml) was added. The mixture was stirred for 15 minutes at room temperature before it was neutralised with 1M acetic acid. Water (20 ml) was added and the resulting mixture was extracted with ethyl acetate (3×5 ml). Solvent was evaporated in vacuo to give the title compound as a brown solid in quantitative yield.

¹H NMR (400 MHz in d₆-DMSO) δ 9.52 (1H, br s, OH), 9.47 (1H, br s, OH), 7.37 (2H, d, J=8.8 Hz, H-2′,6′), 6.90 (1H, d, J=8.1 Hz, H-5), 6.75 (1H, d J=8.8 Hz, H-3′,5′), 6.66 (1H, br s, H-4), 6.27 (1H, dd, J=2.4, 8,1 Hz, H-6), 6.22 (1H, d, J=2.4 Hz, H-8), 5.15 (1H, d, J=5.9 Hz, H-2), 1.90-1.72 (1H, m, CH(CH₃)₂), 0.84 (3H, d, J=6.8 Hz, CH(CH₃)₂), 0.79 (3H, d, J=6.9 Hz, CH(CH₃)₂).

### Example 57

### 2-Butyl-3-(4-hydroxy-phenyl)-2H-chromen-7-ol (35)

### Example 57(a)

### 2-Butyl-7-hydroxy-3-(4-hydroxy-phenyl)-chromen-4-one

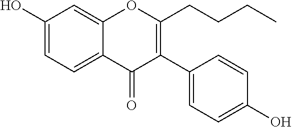

1-(2,4-Dihydroxy-phenyl)-2-(4-hydroxy-phenyl)-ethanone (1.05 g, 4.28 mmol) and valeric anhydride (2.7 ml, 13.7 mmol) were dissolved in triethylamine (10 ml). The solution was heated to reflux for 22 hours. Once the reaction mixture had cooled, it was poured into water (60 ml), acidified (to pH 5) with 2M HCl and stirred at room temperature for two hours. A yellow-brown solid was collected by vacuum filtration and refluxed in methanol (5 ml) with sodium hydroxide solution (2M, 5 ml) for 45 minutes. The mixture was allowed to cool, then poured into water (50 ml), neutralised with 2M HCl, and stirred at room temperature overnight. Vacuum filtration afforded the title compound as a beige solid (Yield: 1.12 g, 84%).

¹H NMR (400 MHz in DMSO) δ 10.81 (1H, br s, OH), 9.50 (1H, br s, OH), 7.80 (1H, d, J=8.7 Hz, H-5), 6.95 (2H, d, J=8.6 Hz, H-2′,6′), 6.84 (1H, dd, J=2.2, 8.7 Hz, H-6), 6.79 (1H, d, J=2.2 Hz, H-8), 6.75 (2H, d, J=8.6 Hz, H-3′,5′), 2.44 (2H, br t, J=7.7 Hz, CH₂CH₂CH₂CH₃), 1.53 (2H, quintet, J=7.7 Hz, CH₂CH₂CH₂CH₃), 1.17 (2H, sextet, J=7.5 Hz, CH₂CH₂CH₂CH₃), 0.72 (3H, t, J=7.3 Hz, CH₂CH₂CH₂CH₃).

### Example 57(b)

### Acetic acid 3-(4-acetoxy-phenyl)-2-butyl-4-oxo-4H-chromen-7-yl ester

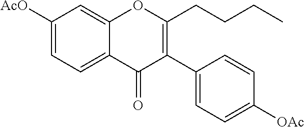

2-Butyl-7-hydroxy-3-(4-hydroxy-phenyl)-chromen-4-one (1.07 g, 3.43 mmol) and potassium carbonate (1.11 g, 8.03 mmol) were refluxed in acetone (15 ml) for one hour. Once cooled, the reaction mixture was poured into water (30 ml) and neutralised with 2M HCl. Vacuum filtration afforded the title compound as a beige solid (Yield: 699 mg, 52%).

¹H NMR (400 MHz in DMSO) δ 8.08 (1H, d, J=8.8 Hz, H-5), 7.54 (1H, d, J=2.2 Hz, H-8), 7.30 (2H, d, J=8.7 Hz, H-2′,6′), 7.28 (1H, dd, J=2.1, 8.6 Hz, H-6), 7.20 (2H, d, J=8.7 Hz, H-3′,5′), 2.54 (2H, br t, J=7.6 Hz, CH₂CH₂CH₂CH₃), 2.33 (3H, s, acetate CH₃), 2.29 (3H, s, acetate CH₃), 1.62 (2H, quintet, J=7.6 Hz, CH₂CH₂CH₂CH₃), 1.24 (2H, sextet, J=7.5 Hz, CH₂CH₂CH₂CH₃), 0.77 (3H, t, J=7.4 Hz, CH₂CH₂CH₂CH₃).

### Example 57(c)

### Acetic acid 3-(4-acetoxy-phenyl)-2-butyl-4-hydroxy-chroman-7-yl ester

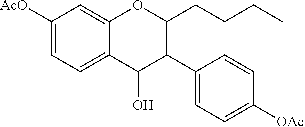

Acetic acid 3-(4-acetoxy-phenyl)-2-butyl-4-oxo-4H-chromen-7-yl ester (435 mg, 1.10 mmol) and 5% palladium on carbon paste (2.42 g) were suspended in ethyl acetate (10 ml). The reaction mixture was stirred under hydrogen (1 bar) for one week. The catalyst was removed via vacuum filtration through a plug of Celite. The solvent was evaporated in vacuo to give the title compound (a mixture of cis and trans isomers around the C-3-C-4 bond) as an off-white solid (Yield: 274 mg, 63%)

¹H NMR (400 MHz, CDCl₃) δ 7.51 (1H, d, J=8.7 Hz, trans H-5), 7.49 (1H, d, J=8.5 Hz, cis H-5), 7.22 (2H, d, J=8.6 Hz, trans H-2′,6′), 7.17 (2H, d, J=8.7 Hz, cis H-2′,6′), 6.99 (2H, d, J=8.7 Hz, cis H-3′,5′), 6.98 (2H, d, J=8.8 Hz, trans H-3′,5′), 6.71 (1H, dd, J=2.3, 8.4 Hz, cis H-6), 6.69 (1H, dd, J=2.3, 8.5 Hz, trans H-6), 6.64 (1H, d, J=2.3 Hz, cis H-8), 6.63 (1H, d, J=2.3 Hz, trans H-8), 5.18 (1H, br dd, J=8.1, 8.1 Hz, cis H-4), 4.94 (1H, br d, J=10.1 Hz, trans H-4), 4.41 (1H, ddd, J=2.3, 5.1, 7.4 Hz, cis H-2), 4.26 (1H, ddd, J=3.2, 7.7, 10.7 Hz, trans H-2), 3.37 (1H, dd, J=2.3, 7.0 Hz, cis H-3), 2.85 (1H, dd, J=10.3, 10.3 Hz, trans H-3), 2.32 (3H, s, trans acetate CH₃), 2.30 (3H, s, cis acetate CH₃), 2.29 (3H, s, trans acetate CH₃), 2.27 (3H, s, cis acetate CH₃), 1.60-1.15 (12H, m, cis and trans CH₂CH₂CH₂CH₃), 0.87 (3H, t, J=7.2 Hz, cis CH₂CH₂CH₂CH₃), 0.82 (3H, t, J=7.3 Hz, trans CH₂CH₂CH₂CH₃).

### Example 57(d)

### Acetic acid 4-(7-acetoxy-2-butyl-2H-chromen-3-yl)-phenyl ester

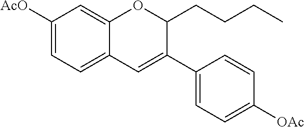

Acetic acid 3-(4-acetoxy-phenyl)-2-butyl-4-hydroxy-chroman-7-yl ester (244 mg, 0.61 mmol) and 85% phosphoric acid (0.75 ml) were refluxed in toluene (7.5 ml) for 19 hours. The reaction mixture was allowed to cool, neutralised with saturated sodium hydrogen carbonate solution and extracted with ethyl acetate (3×20 ml). Solvent was evaporated in vacuo to give the title compound as a brown solid (Yield: 138 mg, 59%).

¹H NMR (400 MHz, CDCl₃) δ 7.46 (2H, d, J=8.9 Hz, H-2′,6′), 7.11 (2H, d, J=8.9 Hz, H-3′,5′), 7.06 (1H, d, J=7.9 Hz, H-5), 6.68 (1H, br s, H-4), 6.66 (1H, dd, J=2.3, 7.9 Hz, H-6), 6.64 (1H, d, J=2.3 Hz, H-8), 5.27 (1H, dd, J=2.5, 9.9 Hz, H-2), 2.32 (3H, s, acetate CH₃), 2.29 (3H, s, acetate CH₃), 1.91-1.79 (1H, m, CH^{a}CH₂CH₂CH₃), 1.52-1.50 (5H, m, CH^{b}CH₂CH₂CH₃), 0.85 (3H, t, J=7.3 Hz, CH₂CH₂CH₂CH₃).

### Example 57(e)

### 2-Butyl-3-(4-hydroxy-phenyl)-2H-chromen-7-ol (35)

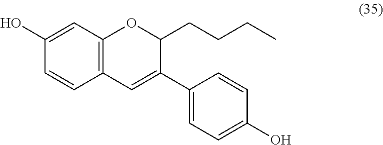

To a solution of acetic acid 4-(7-acetoxy-2-butyl-2H-chromen-3-yl)-phenyl ester (138 mg, 0.13 mmol) in methanol (5 ml), 1M potassium hydroxide solution (0.5 ml) was added. The mixture was stirred for 15 minutes at room temperature before it was neutralised with 1M acetic acid. Water (20 ml) was added and the resulting mixture was extracted with ethyl acetate (3×5 ml). Solvent was evaporated in vacuo to give the title compound as a brown solid in quantitative yield.

¹H NMR (400 MHz in d₆-DMSO) δ 9.56 (1H, br s, OH), 9.51 (1H, br s, OH), 7.35 (2H, d, J=8.7 Hz, H-2′,6′), 6.93 (1H, d, J=8.2 Hz, H-5), 6.78 (2H, d, J=8.7 Hz, H-3′,5′), 6.69 (1H, br s, H-4), 6.32 (1H, dd, J=2.2, 8.1 Hz, H-6), 6.25 (1H, d, J=2.1 Hz, H-8), 5.25 (1H, dd, J=2.5, 9.5 Hz, H-2), 1.77-1.57 (1H, m, CH^{a}CH₂CH₂CH₃), 1.52-1.25 (5H, m, CH^{b}C₂CH₂CH₃), 0.81 (3H, t, J=7.3 Hz, CH₂CH₂CH₂CH₃).

### Example 58

### 3-(4-Hydroxy-phenyl)-2-trifluoromethyl-2H-chromen-7-ol (36)

### Example 58(a)

### 7-Hydroxy-3-(4-hydroxy-phenyl)-2-trifluoromethyl-chromen-4-one

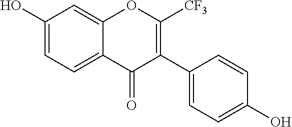

1-(2,4-Dihydroxy-phenyl)-2-(4-hydroxy-phenyl)-ethanone (2.23 g, 9.13 mmol) and trifluoroacetic anhydride (4.0 ml, 28.8 mmol) were dissolved in triethylamine (20 ml). The solution was heated to reflux for one hour. Once the reaction mixture had cooled, it was poured into water (150 ml), acidified (to pH 5) with 2M HCl and stirred at room temperature for two hours. Vacuum filtration afforded the title compound as a brown solid in quantitative yield.

¹H NMR (400 MHz in DMSO) δ 9.84 (1H, br s, OH), 9.60 (1H, br s, OH), 7.89 (1H, d, J=8.7 Hz, H-5), 7.02 (2H, d, J=8.5 Hz, H-2′,6′), 6.97 (1H, dd, J=1.9, 8.7 Hz, H-6), 6.89 (1H, d, J=1.9 Hz, H-8), 6.78 (2H, d, J=8.6 Hz, H-3′,5′).

### Example 58(b)

### Acetic acid 3-(4-acetoxy-phenyl)-4-oxo-2-trifluoromethyl-4H-chromen-7-yl ester

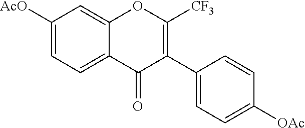

7-Hydroxy-3-(4-hydroxy-phenyl)-2-trifluoromethyl-chromen-4-one (2.92 g, 9.06 mmol) and potassium carbonate (3.04 g, 23 mmol) were refluxed in acetone (15 ml) for thirty minutes. Once cooled, the reaction mixture was poured into water (150 ml) and neutralised with 2M HCl. A brown solid was collect by vacuum filtration. Recrystallisation from ethanol afforded the title compound as an orange-yellow solid (Yield: 1.46 g, 45%).

¹H NMR (400 MHz in CDCl₃) δ 8.25 (1H, d, J=8.8 Hz, H-5), 7.43 (1H, d, J=2.1 Hz, H-8), 7.27 (2H, d, J=8.7 Hz, H-2′,6′), 7.23 (1H, dd, J=2.2, 8.8 Hz, H-6), 7.20 (2H, d, J=8.8 Hz, H-3′,5′), 2.38 (3H, s, acetate CH₃), 2.32 (3H, s, acetate CH₃).

### Example 58(c)

### Acetic acid 3-(4-acetoxy-phenyl)-4-hydroxy-2-trifluoromethyl-chroman-7-yl ester

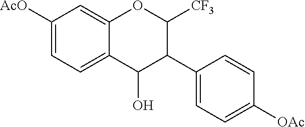

Acetic acid 3-(4-acetoxy-phenyl)-4-oxo-2-trifluoromethyl-4H-chromen-7-yl ester (463 mg, 1.13 mmol) and 5% palladium on carbon paste (2.59 g) were suspended in ethyl acetate (10 ml). The reaction mixture was stirred under hydrogen (1 bar) for one week. The catalyst was removed via vacuum filtration through a plug of Celite. The solvent was evaporated in vacuo to give the title compound (a mixture of cis and trans isomers around the C-3-C-4 bond) as an off-white solid (Yield: 335 mg, 76%)

¹H NMR (400 MHz, CDCl₃) δ 7.54 (1H, d, J=8.4 Hz, trans H-5), 7.53 (1H, d, J=8.3 Hz, cis H-5), 7.17 (2H, d, J=8.6 Hz, cis H-2′,6′), 7.16 (2H, d, J=8.6 Hz, trans H-2′,6′), 7.05 (2H, d, J=9.0 Hz, trans H-3′,5′), 7.00 (2H, d, J=8.8 Hz, cis H-3′5′), 6.83 (1H, dd, J=2.3, 8.4 Hz, cis H-6), 6.80 (1H, dd, J=2.3, 8.4 Hz, trans H-6), 6.80 (1H, d, J=2.2 Hz, cis H-8), 6.76 (1H, d, J=2.2 Hz, trans H-8), 5.25 (1H, br dd, J=7.3, 9.7 Hz, cis H-4), 4.99 (1H, br d, J=10.4 Hz, trans H-4), 4.83 (1H, dq, J=2.5, 6.5 Hz, cis H-2), 4.72 (1H, dq, J=6.5, 11.5 Hz, trans H-2), 3.72 (1H, dd, J=2.5, 6.9 Hz, cis H-3), 3.21 (1H, dd, J=10.3, 10.3 Hz, trans H-3), 2.32 (3H, s, trans acetate CH₃), 2.31 (3H, s, cis acetate CH₃), 2.30 (3H, s, trans acetate CH₃), 2.27 (3H, s, cis acetate CH₃).

### Example 58(d)

### Acetic acid 4-(7-acetoxy-2-trifluoromethyl-2H-chromen-3-yl)-phenyl ester

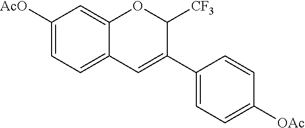

Acetic acid 3-(4-acetoxy-phenyl)-4-hydroxy-2-trifluoromethyl-chroman-7-yl ester (315 mg, 0.80 mmol) and 85% phosphoric acid (0.75 ml) were refluxed in toluene (7.5 ml) for 19 hours. The reaction mixture was allowed to cool, neutralised with saturated sodium hydrogen carbonate solution and extracted with ethyl acetate (3×20 ml). Semi-preparative HPLC gave the title compound as a brown solid (Yield: 49 mg, 17%).

¹H NMR (400 MHz, CDCl₃) δ 7.48 (2H, d, J=8.8 Hz, H-2′,6′), 7.15 (2H, d, J=8.8 Hz, H-3′,5′), 7.14 (1H, d, J=8.0 Hz, H-5), 6.93 (1H, br s, H-4), 6.76 (1H, d, J=2.3 Hz, H-8), 6.75 (1H, dd, 2.5, 7.0 Hz, H-6), 5.68 (1H, quartet, J=6.7 Hz, H-2), 2.32 (3H, s, acetate CH₃), 2.29 (3H, s, acetate CH₃).

### Example 58(e)

### 3-(4-Hydroxy-phenyl)-2-trifluoromethyl-2H-chromen-7-ol (36)

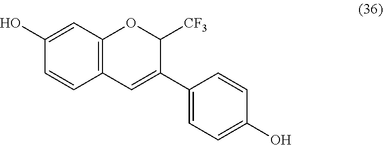

To a solution of Acetic acid 4-(7-acetoxy-2-trifluoromethyl-2H-chromen-3-yl)-phenyl ester (28 mg, 0.08 mmol) in methanol (5 ml), 1M potassium hydroxide solution (0.5 ml) was added. The mixture was stirred for 15 minutes at room temperature before it was neutralised with 1M acetic acid. Water (20 ml) was added and the resulting mixture was extracted with ethyl acetate (3×5 ml). Solvent was evaporated in vacuo to give the title compound as a brown solid in quantitative yield.

¹H NMR (400 MHz in d₆-DMSO) δ 9.80 (1H, br s, OH), 9.63 (1H, br s, OH), 7.48 (2H, d, J=8.8 Hz, H-2′,6′), 7.05 (1H, d, J=8.3 Hz, H-5), 7.03 (1H, br s, H-4), 6.77 (2H, d, J=8.9 Hz, H-3′,5′), 6.40 (1H, dd, J=2.3, 8.2 Hz, H-6), 6.35 (1H, d, J=2.2 Hz), 6.25 (1H, quartet, J=7.4 Hz, H-2).

### Example 59

### 3-(4-Hydroxy-phenyl)-2-phenyl-2H-chromen-7-ol (37)

### Example 59(a)

### 7-Hydroxy-3-(4-hydroxy-phenyl)-2-phenyl-chromen-4-one

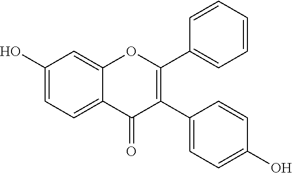

1-(2,4-Dihydroxy-phenyl)-2-(4-hydroxy-phenyl)-ethanone (4.99 g, 20.4 mmol) and benzoic anhydride (15.3 g, 64.5 mmol) were dissolved in triethylamine (10 ml). The solution was heated to reflux for 6 hours. Once the reaction mixture had cooled, it was poured into water (600 ml), acidified (to pH 3) with 2M HCl and stirred at room temperature for two hours. A yellow solid was collected by vacuum filtration and refluxed in methanol (50 ml) with sodium hydroxide solution (2M, 10 ml) for 20 minutes. The mixture was allowed to cool, then poured into water (600 ml), neutralised with 2M HCl, and stirred at room temperature overnight. Vacuum filtration afforded the crude product as a brown solid. Column chromatography afforded the title compound as an orange-yellow solid (Yield: 710 mg, 11%).

¹H NMR (400 MHz in DMSO) δ 10.81 (1H, br s, OH), 9.40 (1H, br s, OH), 7.93 (1H, d, J=8.7 Hz, H-5), 7.43-7.21 (5H, m, Ph Ar—H), 6.94 (1H, dd, J=2.2, 8.7 Hz, H-6), 6.92 (2H, d, J=8.6 Hz, H-2′,6′), 6.90 (1H, d, J=2.2 Hz, H-8), 6.65 (2H, d, J=8.6 Hz, H-3′,5′).

### Example 59(b)

### Acetic acid 3-(4-acetoxy-phenyl)-4-oxo-2-phenyl-4H-chromen-7-yl ester

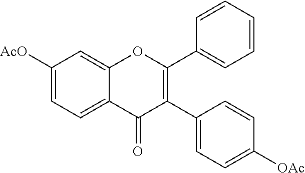

7-Hydroxy-3-(4-hydroxy-phenyl)-2-phenyl-chromen-4-one (710 mg, 2.15 mmol) and potassium carbonate (663 mg, 4.80 mmol) were refluxed in acetone (18 ml) for one hour. Once cooled, the reaction mixture was poured into water (30 ml) and neutralised with 2M HCl. Vacuum filtration afforded the title compound as a beige solid in quantitative yield.

¹H NMR (400 MHz in DMSO) δ 8.14 (1H, d, J=8.5 Hz, H-5), 7.59 (1H, d, J=2.1 Hz, H-8), 7.42-7.30 (6H, m, Ph Ar—H, H-6), 7.19 (2H, d, J=8.7 Hz, H-2′,6′), 7.04 (2H, d, J=8.7 Hz, H-3′,5′), ), 2.32 (3H, s, acetate CH₃), 2.23 (3H, s, acetate CH₃).

### Example 59(c)

### Acetic acid 3-(4-acetoxy-phenyl)-4-hydroxy-2-phenyl-chroman-7-yl ester

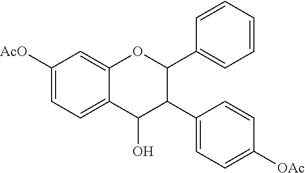

Acetic acid 3-(4-acetoxy-phenyl)-4-oxo-2-phenyl-4H-chromen-7-yl ester (254 mg, 0.61 mmol,) and 5% palladium on carbon paste (1.41 g) were suspended in ethyl acetate (10 ml). The reaction mixture was stirred under hydrogen (1 bar) for 24 hours. The catalyst was removed via vacuum filtration through a plug of Celite. The solvent was evaporated in vacuo to give the title compound as a pale pink solid (Yield: 166 mg, 65%).

¹H NMR (400 MHz, CDCl₃) δ 7.60 (1H, d, J=8.4 Hz, H-5), 7.26-7.10 (5H, m, Ph Ar—H), 6.93 (2H, d, J=8.7 Hz, H-2′,6′), 6.86 (2H, d, J=8.8 Hz, H-3′,5′), 6.81 (1H, dd, J=2.2, 8.4 Hz, H-6), 6.77 (1H, d, J=2.1 Hz, H-8), 5.59 (1H, br d, J=2.1 Hz, H-2), 5.45 (1H, br d, J=7.1 Hz, H-4), 3.65 (1H, dd, J=2.1, 7.0 Hz, H-3), 2.32 (3H, s, acetate CH₃), 2.23 (3H, s, acetate CH₃).

### Example 59(d)

### Acetic acid 3-(4-acetoxy-phenyl)-2-phenyl-2H-chromen-7-yl ester

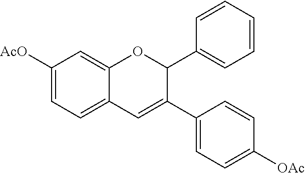

Acetic acid 3-(4-acetoxy-phenyl)-4-hydroxy-2-phenyl-chroman-7-yl ester (160 mg, 0.38 mmol) and 85% phosphoric acid (0.5 ml) were refluxed in toluene (5 ml) for 2 hours. The reaction mixture was allowed to cool, neutralised with saturated sodium hydrogen carbonate solution and extracted with ethyl acetate (3×20 ml). Solvent was evaporated in vacuo to give the title compound as a dark orange solid in quantitative yield.

¹H NMR (400 MHz, CDCl₃) δ 7.39 (2H, d, J=8.8 Hz, H-2′,6′), 7.19-7.15 (5H, m, Ph Ar—H), 7.13 (1H, d, J=8.4 Hz, H-5), 7.06 (1H, br s, H-4), 7.04 (2H, d, J=8.8 Hz, H-3′,5′), 6.64 (1H, dd, J=2.2, 8.2 Hz, H-6), 6.53 (1H, d, J=2.3 Hz, H-8), 6.24 (1H, s, H-2), 2.29 (3H, s, acetate CH₃), 2.23 (3H, s, acetate CH₃).

### Example 59(e)

### 3-(4-Hydroxy-phenyl)-2-phenyl-2H-chromen-7-ol (37)

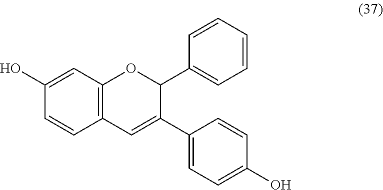

To a solution of acetic acid 3-(4-acetoxy-phenyl)-2-phenyl-2H-chromen-7-yl ester (135 mg) in methanol (5 ml), 1M potassium hydroxide solution (0.5 ml) was added. The mixture was stirred for 10 minutes at room temperature before it was neutralised with 1M acetic acid. Water (20 ml) was added and the resulting mixture was extracted with ethyl acetate (3×20 ml). Solvent was evaporated in vacuo to give the title compound as a brown solid in quantitative yield.

¹H NMR (400 MHz in d₆-DMSO) δ 7.43-7.22 (5H, m, Ph Ar—H), 7.32 (2H, d, J=8.9 Hz, H-2′,6′), 7.05 (1H, br s, H-4), 6.99 (1H, d, J=8.3 Hz, H-5), 6.71 (2H, d, J=8.8 Hz, H-3′,5′), 6.33 (1H, br s, H-2), 6.29 (1H, dd, J=2.3, 8.2 Hz, H-6), 6.12 (1H, d, J=2.4 Hz, H-8).

### Example 60

### 4′,7-Dihydroxy-2-(t-butyl)-isoflav-3-ene

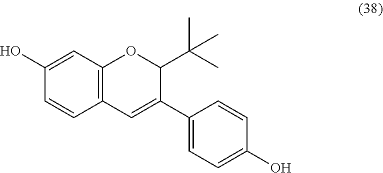

The title compound is prepared according to the general method of Example 54 by reacting trimethylacetic anhydride with 1-(2,4-dihydroxy-phenyl)-2-(4-hydroxy-phenyl)-ethanone and subsequent transformation to the 2-t-butyl isoflav-3-ene compound.

### Example 61

### 4′,7-Dihydroxy-2-(pyridin-2-yl)-isoflav-3-ene

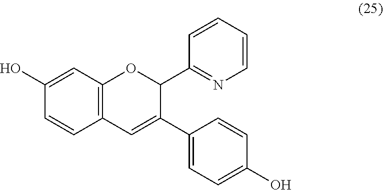

The title compound is prepared according to the general method of Example 54 by reacting pyridine-2-carboxylic acid anhydride with 1-(2,4-dihydroxy-phenyl)-2-(4-hydroxy-phenyl)-ethanone and subsequent transformation to the 2-pyridin-2-yl isoflav-3-ene compound.

In the above general methods, the structures may be optionally substituted or protected with appropriate substituents, or synthons or derivatives thereof. The compounds may be present as, for example, their salts, acetates, benzyl or silyloxy derivatives as can be determined by a skilled synthetic chemist and as generally described herein above. Hydroxy groups can be readily alkylated (MeI/base), acylated (Ac₂O/Py) or silylated (C1 —SiR₃/base) and likewise deprotected by standard methods known in the art.

### 2. Anti-Inflammatory Activity

Prostaglandins e.g PGE₂ and PGI₂ and thromboxanes (TXs) eg TXA₂ are members of a family of fatty acid derivatives known as eicosanoids. They are involved in both normal physiology and inflammatory responses, but have opposing effects on e.g. cytokine release and platelet aggregation. Release of arachidonic acid (AA) from membrane phospholipids provides the primary substrate for eicosanoid synthesis.

Action of the cyclooxygenase (COX) enzymes, regardless of isotype, causes synthesis of the intermediate prostaglandin PGH₂, the common precursor for PGE₂, PGI₂ and TXA₂.

Prostanoids play an important modulatory role in the immune response through complex interactions with leukocytes and parenchymal cells in the inflamed organ. They can produce both pro- and anti-inflammatory actions depending upon the inflammatory stimulus, the predominant prostanoid produced, and the profile of prostanoid receptor expression.

Inhibition of TX synthase leads to reduced formation of TXs, and because there is an increased availability of the substrate PGH₂ for PG synthase, an increase in synthesis of PGs. An increase in PGE₂ can exert anti-inflammatory effects, for example:
a. PGE, has been reported to attenuate some acute inflammatory responses, in particular those initiated by mast cell degranulation.
b. PGE₂ suppresses, whereas TXA₂ increases TNF_{α} and IL-1β. Inhibition of TXA₂ is a potential way of inhibiting inflammatory cytokine production, particularly that of TNF. Currently, biological therapies which suppress TNF levels (with antibodies or soluble TNF receptors have been successful in treating rheumatoid arthritis which is refractory to, or no longer responsive to other therapies. A chemical agent which suppressed TNF production and which could be taken orally would be a great advance Inhibition of TXA₂ formation may be a means of suppressing production of TNF, a cytokine which is involved in the signs and symptoms of joint inflammation and in the longer term degradative phase of joint inflammation manifest in cartilage degradation, diminution of joint space and ultimately, joint failure.
c. PGE₂ inhibits a wide range of T and B cell functions including inhibition of T lymphocyte activation and proliferation and Ig production. Conversely, TXA₂ may promote T cell activation and proliferation and facilitate the development of effector cytolytic T cells (CTLs). Altering this balance in favour of PG production may facilitate ‘quenching’ of an inappropriate immune response as occurs in autoimmune disease.
d. In asthma, PGE₂ promotes vasodilation and increases vascular permeability. As inflammation progresses, PGE₂ synthesis by macrophages is enhanced due to increased expression of COX-2 and PGE-synthase. PGE₂ inhibits leukocyte activation and promotes bronchodilation. TXA₂ synthase inhibitors and thromboxane prostanoid (TP) receptor antagonists have been developed as anti-asthma drugs.
e. In glomerulonephritis there is co-activation of the AA COX pathway toward synthesis of PGs and TX and of lipoxygenase pathways toward synthesis of leukotrienes. TXA₂ is the most abundant eicosanoid synthesized in nephritic glomeruli, and TXA₂ synthase inhibitors (eg Dazmegrel) are now available for the treatment of glomerulonephritis. In a rat model of nephritis, Dazmegrel increased PGE₂ synthesis which is useful as PGE₂ preserves kidney function in glomerulonephritis.
f. Thromboxanes may play a major pathogenic role in inflammatory bowel disease (IBD). TXs are produced in excess not only in inflamed mucosa but also in Crohn's disease by uninflammed bowel and by isolated intestinal and peripheral blood mononuclear cells. Their cellular source is likely to include platelets, neutrophils, endothelial and epithelial cells as well as mononuclear cells. The pro-inflammatory effects of TXs are both direct (diapedesis and activation of neutrophils, mucosal ulceration, reduction of suppressor T-cell activity) and indirect (vasoconstriction, platelet activation). PGs are thought to be protective to gastrointestinal mucosa. Sulfasalazine, a compound frequently administered in the treatment of chronic IBD, as well as one of its main metabolites, sulfapyridine, have been demonstrated to inhibit synthesis of TXB₂ while enhancing synthesis of PGF_{2α} or PGE₂, respectively. In other words, they would appear to have some level of TX synthase inhibition.

### 2.1 Effect on Eicosanoid Synthesis

Eicosanoids, products of the metabolism various fatty acids, the main one of which is arachidonic acid (AA) are involved in both normal physiology and inflammatory responses (vasodilation, coagulation, pain and fever). There are four main families of eicosanoids—the prostaglandins, prostacyclins and the thromboxanes (known collectively as the prostanoids) and the leukotrienes. Two families of enzymes catalyse eicosanoid production:
COX, which generates the prostanoids. COX-1 is responsible for basal prostanoid synthesis, while COX-2 is important in the inflammatory response.
LO which generates the leukotrienes.

Prostanoid synthesis, and thus inflammation can be reduced by inhibiting COX, as is seen with the most prevalent class of anti-inflammatory agents, the NSAIDs (non-steroidal anti-inflammatory drugs). The following assays examined the effects of test compounds for their ability to reduce the synthesis of PGE₂ and TXB₂ produced in response to the inflammatory stimulus of lipopolysaccharide (LPS) in a murine macrophage cell line, RAW 264.7.

Similar patterns of inhibition for production of both PGE2 and TXA2 suggest that a compound is COX inhibitor. On that basis, all compounds demonstrated COX inhibition.

### 2.1.1 Prostanoid Synthesis in a Murine Macrophage Cell Line

### Methods

The mouse macrophage cell line RAW 264.7 was cultured in DMEM supplemented with foetal bovine serum (FBS), 2 mM glutamine and 50 U/ml penicillin/streptomycin (pen/strep). Cells were treated with either test compound (in 0.025% DMSO) or vehicle alone, and added one hour before 50 ng/ml LPS. After incubation for 24 hrs, culture media was collected for PGE₂ or TXB₂ measurement by ELISA (Cayman Chemical). The effect of test compounds at 10 μM on cell viability was examined using an MTT assay.

### Results

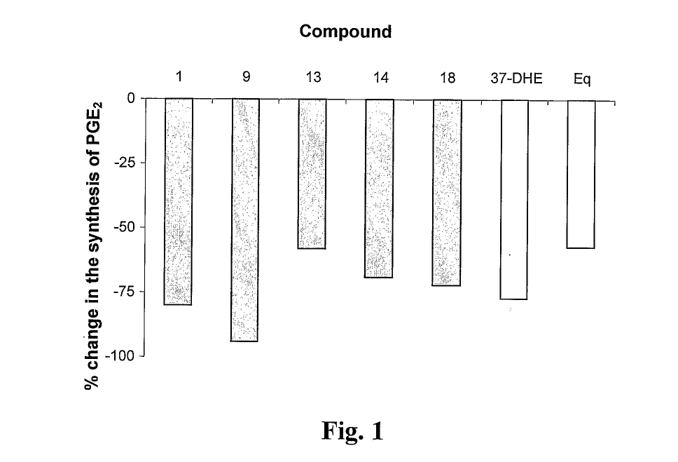

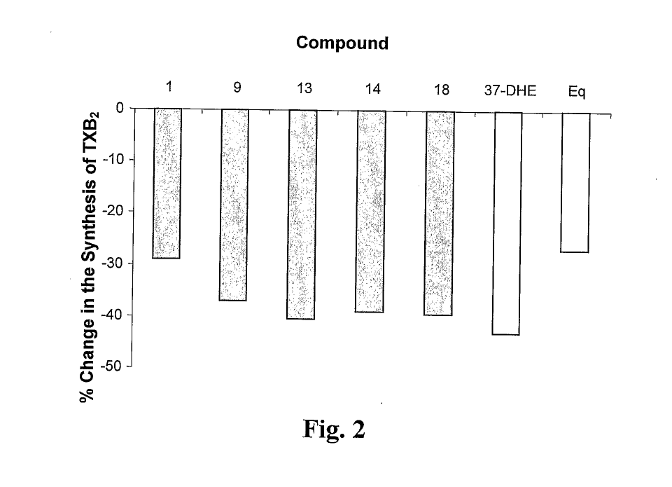

There was no effect on cell viability at 10 μM by any compound except compound (I). It was examined three times and on average, it reduced cell viability by approximately 20%. All compounds substantially reduced the synthesis of PGE₂ and TXB₂ as shown in FIGS. 1 and 2. Comparative results are shown with 3,7-dihydroxyisoflav-3-ene (37-DHE) and equol (Eq).

### 2.2 Effect on Nitric Oxide Production in a Murine Macrophage Cell Line

Nitric oxide (NO), a molecular messenger synthesized by nitric oxide synthase (NOS) from L-arginine and molecular oxygen, is involved in a number of physiological and pathological processes. Three structurally distinct isoforms of NOS have been identified: endothelial (eNOS), inducible (iNOS) and neuronal (nNOS). The site of NO release impacts significantly on its net function and structural impact. Overproduction of NO by mononuclear cells and macrophages in response to iNOS, has been implicated in various inflammatory processes, whereas NO produced by endothelial cells in response to eNOS has a physiological role in maintaining vascular tone (Salerno et al. 2002).

### Methods

Nitrite concentration is a quantitative indicator of NO production (Wang et al. 2002) and was determined by the Griess Reaction (Coligan et al. 1994). Briefly, 100 μL of Griess reagent was added to 50 μL of each supernatant in duplicate in two separate assays, run as for the examination of PGE₂ etc. The absorbance at 550 nm was measured, and nitrite concentrations were determined against a standard curve of sodium nitrite.

### Results

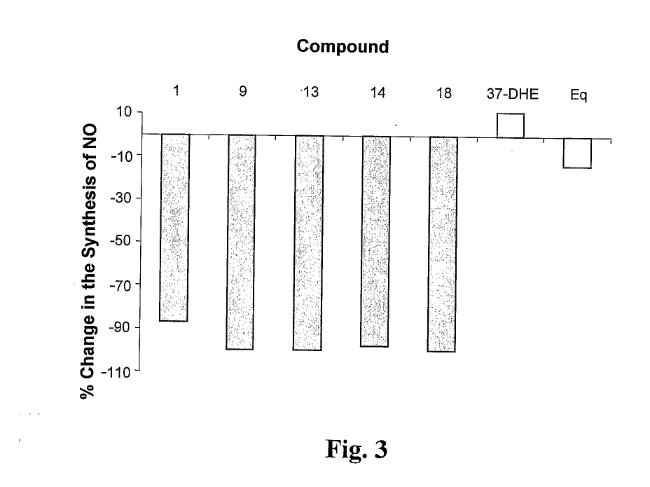

Treatment with all test compounds at 10 μM inhibited the production of nitrite by macrophages stimulated by LPS as shown in FIG. 3. Comparative results are shown with 3,7-dihydroxyisoflav-3-ene (37-DHE) and equol (Eq) which did not inhibit the production of nitrite. This finding confirms the anti-inflammatory activity of the compounds of the invention.

### 2.3 Anti-Oxidant Activity

Reactive oxygen species (ROS) including oxygen ions, peroxides and superoxides are free radicals, small molecules which are capable of damaging cells and DNA via oxidative stress. ROS can initiate lipid peroxidation, direct inhibition of mitochondrial respiratory chain enzymes, inactivation of glyceraldehyde 3-phosphate dehydrogenase, inhibition of membrane sodium/potassium ATP-ase activity, inactivation of membrane sodium channels, and other oxidative modifications of proteins, all of which play a role in the pathophysiology of inflammation (Cuzzocrea 2006). Antioxidants prevent the formation of free radicals, so compounds with antioxidant capabilities can potentially reduce inflammation.

### 2.3.1 Effect on Free Radical Scavenging

The antioxidant (free radical trapping) activity of test compounds was assessed using the stable free radical compound 2,2-diphenyl-1-picrylhydrazyl (DPPH). A stock solution of DPPH was prepared at a concentration of 0.1 mM in ethanol and mixed for 10 minutes prior to use. Test compounds at a concentration of 100 μM were reacted with DPPH for 20 minutes, after which time the absorbance at 517 nm was determined and the change in absorbance compared to a reagent blank (DPPH with ethanol alone). A dose response curve was produced for those compounds with free radical scavenging activity (ΔAbs>0.3) at 100 μM. The IC₅₀ value was estimated as the concentration of test compound that caused a 0.6 change in absorbance (with 1.2 absorbance units representing total scavenging of the DPPH radical). The results are set out in Table 1 below.

TABLE 1
Free radical scavenging ability of test compounds - EC₅₀ (μM)

| Compound | EC₅₀ (μM) |
|---|---|
| 1 | 22.8 |
| 5 | 44.8 |
| 6 | 49.6 |
| 7 | 38.4 |
| 13 | 21.8 |
| 14 | 24.1 |
| 18 | 20 |
| 37-DHE | 55.1 |
| Eq | >100 |

The compounds tested demonstrated an ability to scavenge free radicals and therefore are indicated for reducing inflammation and treatment of related conditions including acting as an antioxidant. Comparative results are shown with 3,7-dihydroxyisoflav-3-ene (37-DHE) which is seen to exhibit only low activity and equol (Eq) which was not active.

### 2.4 Results and Conclusions

Compounds of the invention are shown to inhibit TXB₂ and induce PGE₂ in a dose responsive manner in murine macrophages (RAW 264.7) and human monocytes stimulated with LPS. In addition, compounds of the invention are found to inhibit the induction of TNFα in human monocytes. Accordingly, the compounds of the invention are found to be useful in the treatment of inflammatory diseases and related conditions.

The compounds of the invention are also shown to be antioxidants and can therefore are indicated in the treatment of diseases and disorders responsive to antioxidant activity including inflammation and related conditions. These conditions include cardiovascular indications including myocardial infarction, atherosclerosis, restenosis, stroke, sunlight induced damage, cataracts, arthritis, cancer and other conditions resulting from oxidative damage.

### 3.0 Toxicity to Normal Cells

### 3.1 Method

Neonatal foreskin fibroblasts (NFF), a gift from Dr. Peter Parsons (Queensland Institute of Medical Research) were cultured in RPMI supplemented with 10% FBS (Gibco, Australia), penicillin (100 U/ml), streptomycin (100 mg/ml), L-glutamine (2 mM) and sodium bicarbonate (1.2 g/L), and cultured at 37° C. in a humidified atmosphere of 5% CO₂ for 5 days. Test compounds were added in serial two-fold dilutions from 150 μM in triplicate. These assays were run twice.

In a single assay, RAW 264.7 cells were seeded in 96-well plates at an appropriate cell density as determined from growth kinetics analysis and cultured for 24 hours in the absence and presence of the test compounds. Test compounds were added in serial two-fold dilutions from 150 μM in triplicate.

Cell proliferation was assessed after the addition of 20 μl of 3-4,5 dimethylthiazol-2,5-diphenyl tetrazolium bromide (MTT, 5 mg/ml in PBS, Sigma) for 3 hrs at 37° C. according to manufacturer's instructions, by comparing mean absorbance values of test compounds with that of the control.

### 3.2 Results

Compound 1 was markedly toxic in the tests performed, whereas Compounds 2, 3, 4, 6 and 7 showed only mild to moderate toxicity. Compounds 5 and 8 demonstrated no toxicity in either cell line at the maximum concentration examined (150 μM). The results are set out in Table 2 below.

TABLE 2
Activity of the test compounds against a
panel of normal, non-transformed cells.

| Indication (IC₅₀ μM) | |
|---|---|
| | RAW 264.7 |
| NFF | macrophage |

| Compound | Fibroblast | Unstimulated | with LPS |
|---|---|---|---|
| Compound 1 | 14.72 ± 3.05 | 4.7 | 2.2 |
| Compound 2 | 97.79 ± 1.00 | 15.2 | 90.6 |
| Compound 3 | 140.29 ± 1.07 | 9.3 | 101.7 |
| Compound 4 | 122.15 ± 1.00 | 28.3 | >150 |
| Compound 5 | >150 | >150 | >150 |
| Compound 6 | 138.59 ± 1.05 | >150 | 139.9 |
| Compound 7 | 126.56 ± 1.01 | 98.8 | 115.0 |
| Compound 8 | >150 | >150 | >150 |

Data are presented as IC₅₀ determination (mean ± SD).

### 4.0 Anti-Cancer Activity

### 4.1 Methods

The ovarian cancer cell line, CP70 was obtained as a gift from Dr. Gil Mor (Yale University) and routinely cultured in DMEM/Hams F-12 1:1 (Gibco, Cat#11320-082) supplemented with 10 mM HEPES (Sigma, Cat#H0887), 1× non essential amino acids (Sigma, Cat#M7145), 5.0 g/L sodium bicarbonate (Sigma, Cat#S5761), and 1 mM sodium pyruvate (Sigma, Cat#S8636).

The human pancreatic cancer cell line, HPAC (CRL-2119) was routinely cultured in DMEM/Hams F-12 1:1 (Gibco) and supplemented with 15 mM HEPES, 0.002 mg/ml insulin (Sigma, Cat#I9278), 0.005 mg/ml transferrin (Sigma, Cat#T8158), 40 ng/ml hydrocortisone (Sigma, Cat#H0135) and 10 ng/ml epidermal growth factor (Sigma, Cat#E4269).

The colon adeno-carcinoma cell line HT-29 (HTB-38™) and prostate adenocarcinoma cell line PC-3 (CRL-435™) were cultured in RPMI 1640 medium (Gibco, Cat#21870-076).

The breast cancer cell line MDA-MB-468 (HTB-132™) was cultured in DMEM/Hams F-12 1:1 (Gibco). The melanoma cell line MM200 was obtained as a gift from Peter Hersey (University of Newcastle) and cultured in DMEM medium (Gibco, Cat#11960-069).

The large cell lung cancer cell line NCI-H460 was cultured in RPMI 1640 medium additionally supplemented with 4.5 g/L glucose (Sigma, Cat#G8769), 5.0 g/L sodium pyruvate, 5 g/L sodium bicarbonate and buffered with 10 mM HEPES.

All cultures with the exception of HPAC and CP70 were supplemented with 2 mM L-Glutamine (Gibco, Cat#25030)

All cultures were supplemented with 10% FBS (Gibco, Cat#10099-158), 5000 U/ml penicillin and 5 mg/ml streptomycin (Gibco, Cat#15070), and cultured at 37° C. in a humidified atmosphere of 5% CO₂.

All cell lines were purchased from ATCC (Maryland, USA) except where noted.

IC₅₀ values were determined for each cell line. Cells were seeded in 96-well plates at an appropriate cell density as determined from growth kinetics analysis and cultured for 5 days in the absence and presence of the test compounds. Cell proliferation was assessed after the addition of 20 μl of 3-4,5 dimethylthiazol-2,5-diphenyl tetrazolium bromide (MTT, 2.5 mg/ml in PBS, Sigma) for 3-4 hrs at 37° C. according to manufacturer's instructions. IC₅₀ values were calculated from semi-log plots of % of control proliferation on the y-axis against log dose on the x-axis.

### 4.2 Results

Compound 1 exhibited activity against all cell lines tested (IC₅₀ of ˜3-10 μM). Compounds 2, 4 and 5 were active against MDA-MB-468 and PC-3 cell lines (IC₅₀ ˜2-29 μM). Compound 3 was active against CP70, MDA-MB-468, NCI-H460 and PC-3 cell lines (IC₅₀ ˜5-13 μM). Compounds 6, 7 demonstrated moderate activity against PC-3 and MDA-MB-468 cells (IC₅₀ 14-32 μM). Compound 7 also demonstrated moderate activity against CP70 cells (IC₅₀ ˜31 μM). Compound 8 displayed little activity in the tests performed against the cell lines tested, HPAC, MDA-MB-468 and PC-3. The results are set out in Table 3 below.

TABLE 3
Activity of test compounds against various cell lines representative of different cancer indications

| | | Analogue (IC₅₀ μM) | | | | | | | |
|---|---|---|---|---|---|---|---|---|---|
| | | Geometric Mean ± SD (Log-normal Distribution) | | | | | | | |
| Cancer | | Compound | | | | | | | |
| Indication | Cell ID | 1 | 2 | 3 | 4 | 5 | 6 | 7 | 8 |
| Ovarian | CP70 | 8.39 ± 2.80 | NT | 8.39 ± 1.13 | NT | NT | 72.91 ± 1.07 | 31.06 ± 1.06 | NT |
| Pancreatic | HPAC | 5.21 ± 1.88 | 55.95 ± 1.03 | 59.33 ± 1.28 | 102.76 ± 1.16 | 84.44 ± 1.34 | 74.59 ± 1.24 | 60.51 ± 1.21 | 150.00 ± 1.00 |
| Colorectal | HT-29 | 9.36 ± 2.88 | NT | 110.09 ± 1.03 | NT | NT | 150.00 ± 1.00 | 150.00 ± 1.00 | NT |
| Breast | MDA- | 3.25 ± 1.51 | 2.41 ± 1.26 | 5.18 ± 1.09 | 7.23 ± 1.07 | 16.72 ± 1.18 | 17.02 ± 1.00 | 14.45 ± 1.07 | 66.31 ± 1.05 |
| | MB-468 | | | | | | | | |
| Melanoma | MM200 | 6.37 ± 1.39 | 70.92 ± 1.00 | 69.40 ± 1.22 | NT | NT | 137.75 ± 1.16 | 102.12 ± 1.27 | NT |
| Lung | NCI- | 6.72 ± 1.05 | NT | 12.60 ± 1.10 | NT | 41.07 ± 1.00 | 114.78 ± 1.00 | 51.50 ± 1.03 | NT |
| | H460 | | | | | | | | |
| Prostate | PC-3 | 3.96 ± 2.02 | 4.14 ± 1.04 | 12.02 ± 1.22 | 16.55 ± 1.08 | 29.03 ± 1.05 | 31.69 ± 1.05 | 31.89 ± 1.47 | 78.71 ± 1.26 |

Likewise, compounds 9 to 38 are shown to have from moderate to very good and excellent activity across a number of cancer cell lines.

Pharmacophore studies on 5AR and α_{1A} adrenoreceptor activity using CATALYST from Accelrys show that the 3-ene compounds of the invention, especially compounds (1)-(3), (5)-(7) and (9)-(38), and more especially compound (12) have particular activity. Thus the compounds find utility in treating or ameliorating symptoms associated with prostrate enlargement such as and including partial blockage of the urethra, pain and discomfort in the prostrate region including pain during urination or ejaculation, cell proliferation and cancer.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited herein, if any, are hereby incorporated by reference.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

### SELECTED REFERENCES

Coligan, J. E., A. M. Kruisbeek, et al., Eds. (1994). Oxidative Metabolism of Murine Macrophages. Current Protocols in Immunology. U.S.A., John Wiley and Sons, Inc.
Cuzzocrea, S. (2006). “Role of nitric oxide and reactive oxygen species in arthritis.” Curr Pharm Des 12(27): 3551-70.
Salerno, L., V. Sorrenti, et al. (2002). “Progress in the development of selective nitric oxide synthase (NOS) inhibitors.” Current Pharmaceutical Design. 8(3): 177-200.
Wang, J. and G. Mazza (2002). “Inhibitory effects of anthocyanins and other phenolic compounds on nitric oxide production in LPS/IFN-gamma-activated RAW 264.7 macrophages.” Journal of Agricultural & Food Chemistry 50(4): 850-7.

1-4. (canceled)

5. A method for the treatment, prevention or amelioration of an oxidative or inflammatory disease or disorder, which comprises administering to a subject one or more compounds of formula (I) or a pharmaceutically acceptable salt or derivative thereof, 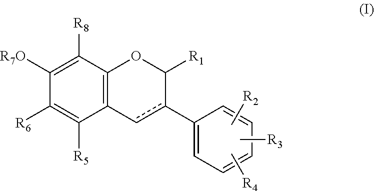
wherein
R₁ is hydroxy, OR₉, OC(O)R₉, OSi(R₁₀)₃, alkyl, cycloalkyl, aminoalkyl, —NR₁₁(R₁₂), R₁₁(R₁₂)N-alkyl, aryl, arylalkyl, thiol, alkylthio, nitro, cyano, halo, alkenyl, alkynyl, heteroaryl, arylalkylamino or alkylaryl,
R₂, R₃ and R₄ are independently hydrogen, hydroxy, OR₉, OC(O)R₉, OSi(R₁₀)₃, alkyl, cycloalkyl, aryl, arylalkyl, thiol, alkylthio, nitro, cyano or halo,
R₅ and R₆ are independently hydrogen, hydroxy, OR₉, OC(O)R₉ or alkyl,
R₇ is hydrogen, alkyl, haloalkyl, C(O)R₉, Si(R₁₀)₃, cycloalkyl, aryl or arylalkyl,
R₈ is hydrogen, hydroxy, OR₉, OC(O)R₉, alkyl, cycloalkyl, aryl, arylalkyl, nitro, cyano or halo,
R₉ is alkyl, haloalkyl, aryl or arylalkyl,
R₁₀ is independently alkyl or aryl,
R₁₁ and R₁₂ are independently hydrogen, alkyl, arylalkyl, aryl or BOC, or together form with the nitrogen atom to which they are attached a heterocyclic ring, and
the drawing [symbol] represents either a single bond or a double bond,
which hydrocarbon substituents can be optionally substituted by one or more of alkyl, halo, acyloxy, hydroxy, halo, alkoxy, silyloxy, nitro and cyano.

6. (canceled)

7. A compound of general formula (I): 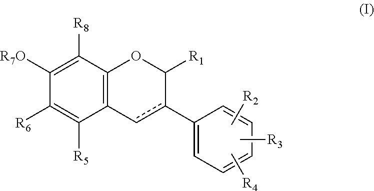
wherein
R₁ is hydroxy, OR₉, OC(O)R₉, alkyl, cycloalkyl, aminoalkyl, —NR₁₁(R₁₂), R₁₁(R₁₂)N-alkyl, aryl, arylalkyl, thiol, alkylthio, nitro, cyano, halo, alkenyl, alkynyl, heteroaryl, arylalkylamino or alkylaryl,
R₂, R₃ and R₄ are independently hydrogen, hydroxy, OR₉, OC(O)R₉, alkyl, cycloalkyl, aryl, arylalkyl, thiol, alkylthio, nitro, cyano or halo,
R₅ and R₆ are independently hydrogen, hydroxy, OR₉, OC(O)R₉ or alkyl,
R₇ is hydrogen,
R₈ is hydrogen, hydroxy, OR₉, OC(O)R₉, alkyl, cycloalkyl, aryl, arylalkyl, nitro, cyano or halo,
R₉ is alkyl, haloalkyl, aryl or arylalkyl,
R₁₁ and R₁₂ are independently hydrogen, alkyl, arylalkyl, aryl or BOC, or together form with the nitrogen atom to which they are attached a heterocyclic ring, and
the drawing [symbol] represents either a single bond or a double bond, preferably a double bond
which hydrocarbon substituents can be optionally substituted by one or more of alkyl, halo, acyloxy, hydroxy, halo, alkoxy, silyloxy, nitro and cyano, and
which compounds include pharmaceutically acceptable salts thereof,
with the proviso that the following compounds are specifically excluded:
2,4′,7-trihydroxyisoflavan,
2,3-bis(4-hydroxyphenyl)-2H-1-benzopyran-7-ol,
3-(3,4-dihydroxyphenyl)-2-(2,4,6-trihydroxyphenyl)-5,7-chromandiol,
3,4-dihydro-2-methyl-3-phenyl-2H-1-benzopyran-7-ol,
2-methyl-4′,7-dihydroxyisoflav-3-ene,
2-ethyl-4′,7-dihydroxyisoflav-3-ene,
2-isopropyl-4′,7-dihydroxyisoflav-3-ene,
2-phenyl-4′,7-dihydroxyisoflav-3-ene,
2-(4-fluorophenyl)-4′,7-dihydroxyisoflav-3-ene,
2-(4-anisyl)-4′,7-dihydroxyisoflav-3-ene,
2-naphthyl-4′,7-dihydroxyisoflav-3-ene,
2-thienyl-4′,7-dihydroxyisoflav-3-ene,
2-vinyl-4′,7-dihydroxyisoflav-3-ene,
2-(4-hydroxyphenyl)-3-phenyl-7-methoxy-2H-1-benzopyran,
2-(N-n-butyl-N-methyl-10-aminodecyl)-3(4-hydroxyphenyl)-7-hydroxy-2H-1-benzopyran, and
2-(N-n-butyl-N-methyl-11-aminoundecyl)-3(4-hydroxyphenyl)-7-hydroxy-2H-1-benzopyran.

8. The compound of claim 7, which is of the formula (I-1): 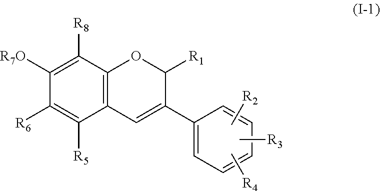
wherein
R₁, R₂, R₃, R₄, R₅, R₆, R₇ and R₈ are as defined in claim 7.

9. The compound of claim 7, wherein R₁ is an alkyl group selected from propyl, n-butyl and t-butyl.

10. The compound of claim 7, wherein R₁ is a haloalkyl group selected from trifluoromethyl.

11. The compound of claim 7, wherein R₁ is an aminoalkyl group selected from aminomethyl.

12. The compound of claim 7, wherein R₁ is an alkenyl group selected from allyl.

13. The compound of claim 7, wherein R₁ is an alkynyl group selected from ethynyl.

14. The compound of claim 7, wherein R₁ is an alkoxy group selected from methoxy, ethoxy and bromopropoxy.

15. The compound of claim 7, wherein R₁ is an amino group selected from benzylamino.

16. The compound of claim 7, wherein R₁ is cyano.

17. The compound of claim 7, wherein R₁ is hydroxy.

18. The compound of claim 7, wherein R₁ is an alkylthio group selected from methylthio and ethylthio.

19. The compound of claim 7, wherein R₁ is a heteroaryl group selected from thiazolyl, triazolyl, pyridinyl, pyridazyl, pyrimidinyl, pyrazinyl, pyrrolyl, imidazyl, triazolyl, tetrazolyl, triazinyl and tetrazinyl.

20. The compound of claim 7, wherein
R₂, R₃ and R₄ are independently hydrogen, hydroxy, OR₉, OC(O)R₉ or halo.

21. The compound of claim 20, wherein
R₂, R₃ and R₄ are independently hydrogen, hydroxy, OMe or OC(O)Me

22. The compound of claim 7, wherein the compound is of formula (I-2): 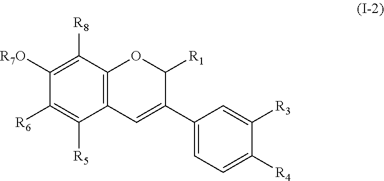
wherein
R₃ and R₄ are independently hydrogen, hydroxy, methoxy or OC(O)Me.

23. (canceled)

24. The compound of claim 22, wherein
one of R₃ and R₄ is hydroxy and the other is hydrogen.

25. The compound of claim 8, wherein
R₅, R₆ and R₈ are independently hydrogen, hydroxy or methyl.

26. (canceled)

27. The compound of claim 25, wherein
R₅, R₆ and R₈ are hydrogen.

28-29. (canceled)

30. The compound of claim 19, wherein
R₁ is pyridyl, pyrimidinyl, pyrazinyl or pyridazinyl.

31. The compound of claim 7 selected from compounds (1), (4)-(33), (35)-(36) and (38): 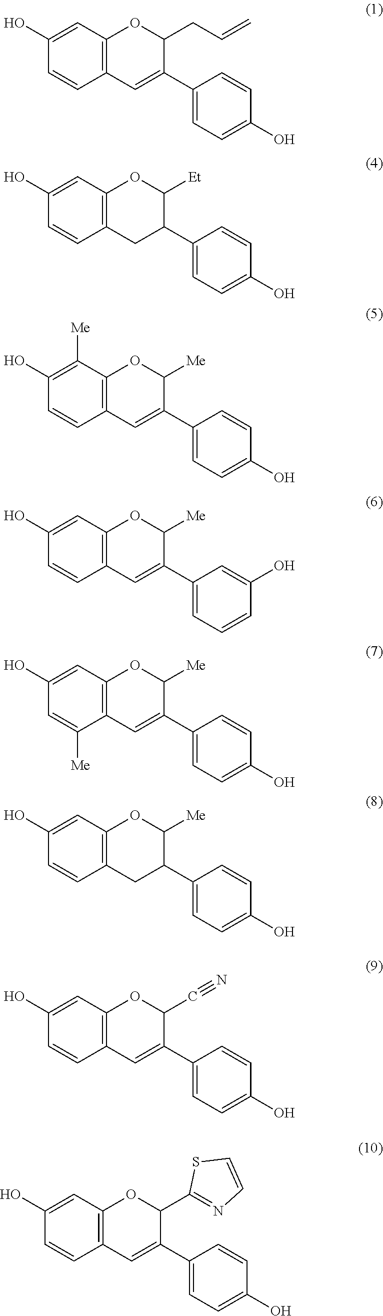 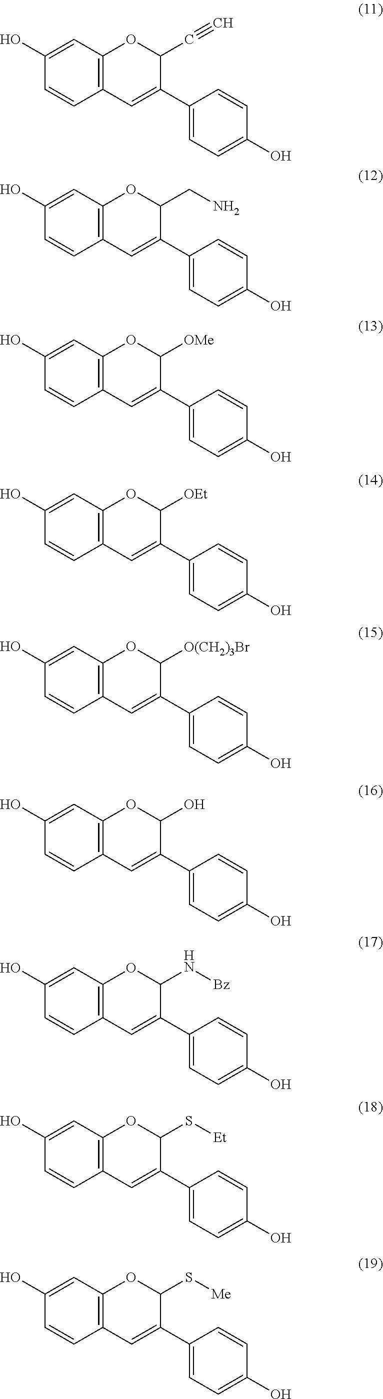 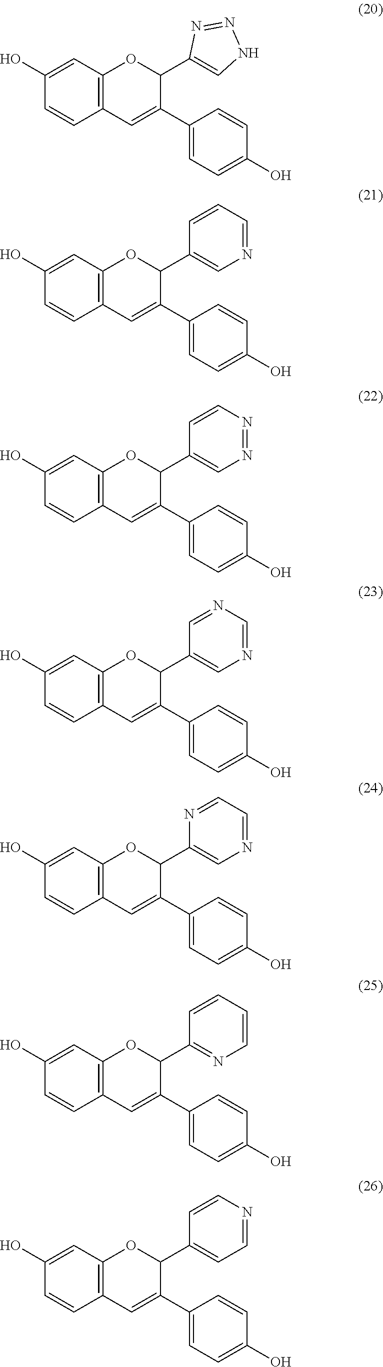 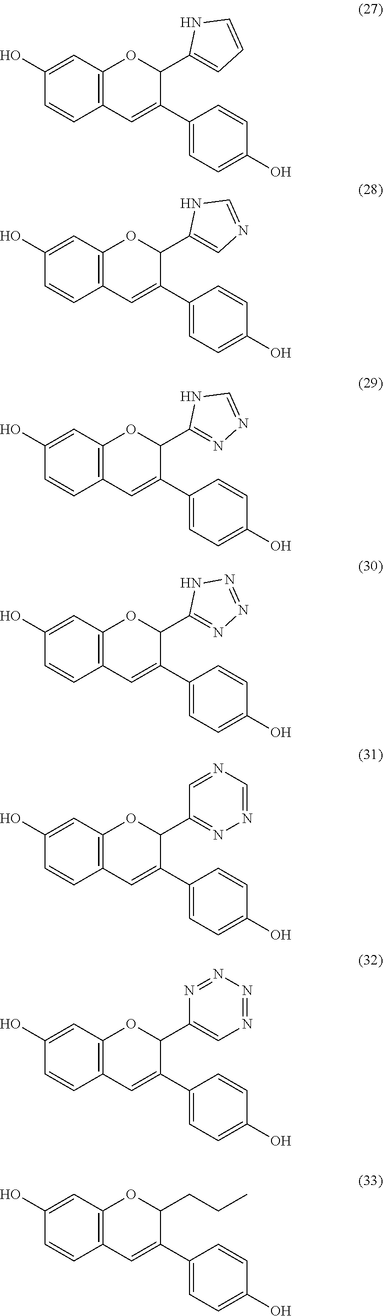 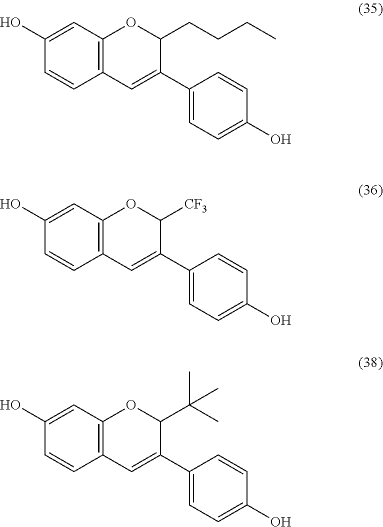
or a pharmaceutically acceptable salt thereof.

32. A pharmaceutical composition which comprises one or more compounds of formula (I) as defined in claim 7, or a pharmaceutically acceptable salt thereof, in association with one or more pharmaceutical carriers, excipients, auxiliaries and/or diluents.

33. (canceled)

34. The method of claim 5, wherein the inflammatory disease or disorder is selected from osteoarthritis, inflammatory bowel disease (ulcerative colitis and Crohn's disease), ulcerative proctitis, distal colitis, autoimmune disorders (SLE, rheumatoid arthritis, glomerulonephritis), asthma and diseases involving pulmonary inflammation, cardiovascular disorders including atherosclerosis, hypertension and lipid dyscrasias.

35. The method of claim 5, for the treatment, prevention or amelioration of an oxidative disease or disorder.
